SEQUENCE LISTING (579 sequences)

SEQ ID NO: 1 - protein, 8 aa, Homo sapiens
FLNVLSPR

SEQ ID NO: 2 - protein, 8 aa, Homo sapiens
AEIEYLEK

SEQ ID NO: 3 - protein, 9 aa, Homo sapiens
FPEVDVLTK

SEQ ID NO: 4 - protein, 7 aa, Homo sapiens
DLIQHPK

SEQ ID NO: 5 - protein, 9 aa, Homo sapiens
GDPGLIGPK

SEQ ID NO: 6 - protein, 11 aa, Homo sapiens
NFPSPVDAAFR

SEQ ID NO: 7 - protein, 12 aa, Homo sapiens
LVNVVLGAHNVR

SEQ ID NO: 8 - protein, 9 aa, Homo sapiens
ILIGTVFHK

SEQ ID NO: 9 - protein, 10 aa, Homo sapiens
AEVDDVIQVR

SEQ ID NO: 10 - protein, 9 aa, Homo sapiens
TGIVSGFGR

SEQ ID NO: 11 - protein, 12 aa, Homo sapiens
AAAATGTIFTFR

SEQ ID NO: 12 - protein, 9 aa, Homo sapiens
TLEAQLTPR

SEQ ID NO: 13 - protein, 13 aa, Homo sapiens
ILAGPAGDSNVVK

SEQ ID NO: 14 - protein, 10 aa, Homo sapiens
GPDPSSPAFR

SEQ ID NO: 15 - protein, 12 aa, Homo sapiens
DEIPHNDIALLK

SEQ ID NO: 16 - protein, 9 aa, Homo sapiens
SALVLQYLR

SEQ ID NO: 17 - protein, 9 aa, Homo sapiens
HINIDQFVR

SEQ ID NO: 18 - protein, 13 aa, Homo sapiens
LGVEFDETTADDR

SEQ ID NO: 19 - protein, 10 aa, Homo sapiens
ITDYGLESFR

SEQ ID NO: 20 - protein, 9 aa, Homo sapiens
PLQALLDGR

SEQ ID NO: 21 - protein, 20 aa, Homo sapiens
AYLLPAPPAPGNASESEEDR

SEQ ID NO: 22 - protein, 15 aa, Homo sapiens
SAGSVESPSVSSTHR

SEQ ID NO: 23 - protein, 7 aa, Homo sapiens
FHPLHSK

SEQ ID NO: 24 - protein, 18 aa, Homo sapiens
VDYESQSTDTQNFSSESK

SEQ ID NO: 25 - protein, 11 aa, Homo sapiens
YGQPLPGYTTK

SEQ ID NO: 26 - protein, 20 aa, Homo sapiens
YTYNYEAESSSGVPGTADSR

SEQ ID NO: 27 - protein, 10 aa, Homo sapiens
EVYGFNPEGK

SEQ ID NO: 28 - protein, 7 aa, Homo sapiens
LAIPEGK

SEQ ID NO: 29 - protein, 8 aa, Homo sapiens
QVFLYPEK

SEQ ID NO: 30 - protein, 10 aa, Homo sapiens
DEPTYILNIK

SEQ ID NO: 31 - protein, 16 aa, Homo sapiens
GIISALLVPPETEEAK

SEQ ID NO: 32 - protein, 11 aa, Homo sapiens
GNVATEISTER

SEQ ID NO: 33 - protein, 10 aa, Homo sapiens
TGISPLALIK

SEQ ID NO: 34 - protein, 11 aa, Homo sapiens
EQHLFLPFSYK

SEQ ID NO: 35 - protein, 6 aa, Homo sapiens
LEDTPK

SEQ ID NO: 36 - protein, 7 aa, Homo sapiens
FFGEGTK

SEQ ID NO: 37 - protein, 7 aa, Homo sapiens
QAEAVLK

SEQ ID NO: 38 - protein, 10 aa, Homo sapiens
LTISEQNIQR

SEQ ID NO: 39 - protein, 15 aa, Homo sapiens
ATLYALSHAVNNYHK

SEQ ID NO: 40 - protein, 7 aa, Homo sapiens
AAIQALR

SEQ ID NO: 41 - protein, 9 aa, Homo sapiens
SPSQADINK

SEQ ID NO: 42 - protein, 14 aa, Homo sapiens
IVQILPWEQNEQVK

SEQ ID NO: 43 - protein, 22 aa, Homo sapiens
NFVASHIANILNSEELDIQDLK

SEQ ID NO: 44 - protein, 6 aa, Homo sapiens
NYQLYK

SEQ ID NO: 45 - protein, 13 aa, Homo sapiens
SVSLPSLDPASAK

SEQ ID NO: 46 - protein, 15 aa, Homo sapiens
IEGNLIFDPNNYLPK

SEQ ID NO: 47 - protein, 21 aa, Homo sapiens
TTLTAFGFASADLIEIGLEGK

SEQ ID NO: 48 - protein, 12 aa, Homo sapiens
GFEPTLEALFGK

SEQ ID NO: 49 - protein, 10 aa, Homo sapiens
QGFFPDSVNK

SEQ ID NO: 50 - protein, 15 aa, Homo sapiens
ALYWVNGQVPDGVSK

SEQ ID NO: 51 - protein, 10 aa, Homo sapiens
VLVDHFGYTK

SEQ ID NO: 52 - protein, 19 aa, Homo sapiens
ILGEELGFASLHDLQLLGK

SEQ ID NO: 53 - protein, 7 aa, Homo sapiens
FIIPSPK

SEQ ID NO: 54 - protein, 15 aa, Homo sapiens
LLSGGNTLHLVSTTK

SEQ ID NO: 55 - protein, 11 aa, Homo sapiens
TEVIPPLIENR

SEQ ID NO: 56 - protein, 7 aa, Homo sapiens
ALVDTLK

SEQ ID NO: 57 - protein, 9 aa, Homo sapiens
FVTQAEGAK

SEQ ID NO: 58 - protein, 9 aa, Homo sapiens
VNDESTEGK

SEQ ID NO: 59 - protein, 8 aa, Homo sapiens
LTLDIQNK

SEQ ID NO: 60 - protein, 7 aa, Homo sapiens
GVISIPR

SEQ ID NO: 61 - protein, 11 aa, Homo sapiens
SEILAHWSPAK

SEQ ID NO: 62 - protein, 9 aa, Homo sapiens
VAWHYDEEK

SEQ ID NO: 63 - protein, 14 aa, Homo sapiens
IEFEWNTGTNVDTK

SEQ ID NO: 64 - protein, 19 aa, Homo sapiens
ASGSLPYTQTLQDHLNSLK

SEQ ID NO: 65 - protein, 10 aa, Homo sapiens
IEIPLPFGGK

SEQ ID NO: 66 - protein, 7 aa, Homo sapiens
TPALHFK

SEQ ID NO: 67 - protein, 9 aa, Homo sapiens
SVGFHLPSR

SEQ ID NO: 68 - protein, 11 aa, Homo sapiens
EFQVPTFTIPK

SEQ ID NO: 69 - protein, 23 aa, Homo sapiens
ADSVVDLLSYNVQGSGETTYDHK

SEQ ID NO: 70 - protein, 12 aa, Homo sapiens
NTFTLSYDGSLR

SEQ ID NO: 71 - protein, 7 aa, Homo sapiens
FLDSNIK

SEQ ID NO: 72 - protein, 6 aa, Homo sapiens
FSHVEK

SEQ ID NO: 73 - protein, 8 aa, Homo sapiens
LGNNPVSK

SEQ ID NO: 74 - protein, 6 aa, Homo sapiens
QHLFVK

SEQ ID NO: 75 - protein, 6 aa, Homo sapiens
IDGQFR

SEQ ID NO: 76 - protein, 7 aa, Homo sapiens
VSSFYAK

SEQ ID NO: 77 - protein, 8 aa, Homo sapiens
LNGESNLR

SEQ ID NO: 78 - protein, 15 aa, Homo sapiens
FNSSYLQGTNQITGR

SEQ ID NO: 79 - protein, 20 aa, Homo sapiens
YEDGTLSLTSTSDLQSGIIK

SEQ ID NO: 80 - protein, 9 aa, Homo sapiens
YENYELTLK

SEQ ID NO: 81 - protein, 6 aa, Homo sapiens
SDTNGK

SEQ ID NO: 82 - protein, 7 aa, Homo sapiens
NFATSNK

SEQ ID NO: 83 - protein, 6 aa, Homo sapiens
QNALLR

SEQ ID NO: 84 - protein, 11 aa, Homo sapiens
SEYQADYESLR

SEQ ID NO: 85 - protein, 25 aa, Homo sapiens
FFSLLSGSLNSHGLELNADILGTDK

SEQ ID NO: 86 - protein, 7 aa, Homo sapiens
INSGAHK

SEQ ID NO: 87 - protein, 15 aa, Homo sapiens
IGQDGISTSATTNLK

SEQ ID NO: 88 - protein, 6 aa, Homo sapiens
LTTNGR

SEQ ID NO: 89 - protein, 6 aa, Homo sapiens
FSLDGK

SEQ ID NO: 90 - protein, 6 aa, Homo sapiens
NIFNFK

SEQ ID NO: 91 - protein, 7 aa, Homo sapiens
VSQEGLK

SEQ ID NO: 92 - protein, 19 aa, Homo sapiens
FDHTNSLNIAGLSLDFSSK

SEQ ID NO: 93 - protein, 9 aa, Homo sapiens
LDNIYSSDK

SEQ ID NO: 94 - protein, 21 aa, Homo sapiens
QTVNLQLQPYSLVTTLNSDLK

SEQ ID NO: 95 - protein, 11 aa, Homo sapiens
YNALDLTNNGK

SEQ ID NO: 96 - protein, 8 aa, Homo sapiens
LHVAGNLK

SEQ ID NO: 97 - protein, 9 aa, Homo sapiens
GAYQNNEIK

SEQ ID NO: 98 - protein, 15 aa, Homo sapiens
HIYAISSAALSASYK

SEQ ID NO: 99 - protein, 6 aa, Homo sapiens
ADTVAK

SEQ ID NO: 100 - protein, 9 aa, Homo sapiens
VQGVEFSHR

SEQ ID NO: 101 - protein, 14 aa, Homo sapiens
LALWGEHTGQLYSK

SEQ ID NO: 102 - protein, 13 aa, Homo sapiens
AEPLAFTFSHDYK

SEQ ID NO: 103 - protein, 10 aa, Homo sapiens
GSTSHHLVSR

SEQ ID NO: 104 - protein, 9 aa, Homo sapiens
SISAALEHK

SEQ ID NO: 105 - protein, 15 aa, Homo sapiens
VSALLTPAEQTGTWK

SEQ ID NO: 106 - protein, 18 aa, Homo sapiens
TQFNNNEYSQDLDAYNTK

SEQ ID NO: 107 - protein, 10 aa, Homo sapiens
VDTVDPPYPR

SEQ ID NO: 108 - protein, 8 aa, Homo sapiens
IGVELTGR

SEQ ID NO: 109 - protein, 13 aa, Homo sapiens
TLADLTLLDSPIK

SEQ ID NO: 110 - protein, 21 aa, Homo sapiens
NQDVHSINLPFFETLQEYFER

SEQ ID NO: 111 - protein, 12 aa, Homo sapiens
QTIIVVLENVQR

SEQ ID NO: 112 - protein, 16 aa, Homo sapiens
LPQQANDYLNSFNWER

SEQ ID NO: 113 - protein, 6 aa, Homo sapiens
QVSHAK

SEQ ID NO: 114 - protein, 6 aa, Homo sapiens
LTALTK

SEQ ID NO: 115 - protein, 14 aa, Homo sapiens
ITENDIQIALDDAK

SEQ ID NO: 116 - protein, 9 aa, Homo sapiens
DSYDLHDLK

SEQ ID NO: 117 - protein, 13 aa, Homo sapiens
IAIANIIDEIIEK

SEQ ID NO: 118 - protein, 9 aa, Homo sapiens
SLDEHYHIR

SEQ ID NO: 119 - protein, 16 aa, Homo sapiens
TIHDLHLFIENIDFNK

SEQ ID NO: 120 - protein, 15 aa, Homo sapiens
SGSSTASWIQNVDTK

SEQ ID NO: 121 - protein, 6 aa, Homo sapiens
IQIQEK

SEQ ID NO: 122 - protein, 13 aa, Homo sapiens
HIQNIDIQHLAGK

SEQ ID NO: 123 - protein, 9 aa, Homo sapiens
QHIEAIDVR

SEQ ID NO: 124 - protein, 14 aa, Homo sapiens
VLLDQLGTTISFER

SEQ ID NO: 125 - protein, 9 aa, Homo sapiens
INDILEHVK

SEQ ID NO: 126 - protein, 15 aa, Homo sapiens
HFVINLIGDFEVAEK

SEQ ID NO: 127 - protein, 7 aa, Homo sapiens
VHELIER

SEQ ID NO: 128 - protein, 9 aa, Homo sapiens
LVELAHQYK

SEQ ID NO: 129 - protein, 9 aa, Homo sapiens
LSNVLQQVK

SEQ ID NO: 130 - protein, 10 aa, Homo sapiens
LVGFIDDAVK

SEQ ID NO: 131 - protein, 7 aa, Homo sapiens
LNELSFK

SEQ ID NO: 132 - protein, 8 aa, Homo sapiens
TFIEDVNK

SEQ ID NO: 133 - protein, 14 aa, Homo sapiens
SFDYHQFVDETNDK

SEQ ID NO: 134 - protein, 13 aa, Homo sapiens
LNGEIQALELPQK

SEQ ID NO: 135 - protein, 7 aa, Homo sapiens
LFLEETK

SEQ ID NO: 136 - protein, 14 aa, Homo sapiens
ATVAVYLESLQDTK

SEQ ID NO: 137 - protein, 7 aa, Homo sapiens
ETLEDTR

SEQ ID NO: 138 - protein, 25 aa, Homo sapiens
YLSLVGQVYSTLVTYISDWWTLAAK

SEQ ID NO: 139 - protein, 16 aa, Homo sapiens
NLTDFAEQYSIQDWAK

SEQ ID NO: 140 - protein, 13 aa, Homo sapiens
ALVEQGFTVPEIK

SEQ ID NO: 141 - protein, 16 aa, Homo sapiens
ATFQTPDFIVPLTDLR

SEQ ID NO: 142 - protein, 9 aa, Homo sapiens
IPSVQINFK

SEQ ID NO: 143 - protein, 8 aa, Homo sapiens
VEDIPLAR

SEQ ID NO: 144 - protein, 7 aa, Homo sapiens
ITLPDFR

SEQ ID NO: 145 - protein, 6 aa, Homo sapiens
LYSILK

SEQ ID NO: 146 - protein, 18 aa, Homo sapiens
LEVLNFDFQANAQLSNPK

SEQ ID NO: 147 - protein, 7 aa, Homo sapiens
INPLALK

SEQ ID NO: 148 - protein, 11 aa, Homo sapiens
SNTVASLHTEK

SEQ ID NO: 149 - protein, 12 aa, Homo sapiens
NTLELSNGVIVK

SEQ ID NO: 150 - protein, 12 aa, Homo sapiens
INNQLTLDSNTK

SEQ ID NO: 151 - protein, 10 aa, Homo sapiens
LDFSSQADLR

SEQ ID NO: 152 - protein, 11 aa, Homo sapiens
AGHIAWTSSGK

SEQ ID NO: 153 - protein, 16 aa, Homo sapiens
VNQNLVYESGSLNFSK

SEQ ID NO: 154 - protein, 20 aa, Homo sapiens
LEIQSQVDSQHVGHSVLTAK

SEQ ID NO: 155 - protein, 6 aa, Homo sapiens
AEFTGR

SEQ ID NO: 156 - protein, 8 aa, Homo sapiens
HDAHLNGK

SEQ ID NO: 157 - protein, 6 aa, Homo sapiens
VIGTLK

SEQ ID NO: 158 - protein, 23 aa, Homo sapiens
NSLFFSAQPFEITASTNNEGNLK

SEQ ID NO: 159 - protein, 25 aa, Homo sapiens
IDFLNNYALFLSPSAQQASWQVSAR

SEQ ID NO: 160 - protein, 12 aa, Homo sapiens
LPYTIITTPPLK

SEQ ID NO: 161 - protein, 7 aa, Homo sapiens
DFSLWEK

SEQ ID NO: 162 - protein, 8 aa, Homo sapiens
QSFDLSVK

SEQ ID NO: 163 - protein, 9 aa, Homo sapiens
NNALDFVTK

SEQ ID NO: 164 - protein, 6 aa, Homo sapiens
SYNETK

SEQ ID NO: 165 - protein, 7 aa, Homo sapiens
SHDELPR

SEQ ID NO: 166 - protein, 20 aa, Homo sapiens
VPSYTLILPSLELPVLHVPR

SEQ ID NO: 167 - protein, 7 aa, Homo sapiens
LSLPDFK

SEQ ID NO: 168 - protein, 10 aa, Homo sapiens
LATALSLSNK

SEQ ID NO: 169 - protein, 15 aa, Homo sapiens
FVEGSHNSTVSLTTK

SEQ ID NO: 170 - protein, 7 aa, Homo sapiens
AQIPILR

SEQ ID NO: 171 - protein, 8 aa, Homo sapiens
QELNGNTK

SEQ ID NO: 172 - protein, 6 aa, Homo sapiens
GAVDHK

SEQ ID NO: 173 - protein, 17 aa, Homo sapiens
LSLESLTSYFSIESSTK

SEQ ID NO: 174 - protein, 17 aa, Homo sapiens
EYSGTIASEANTYLNSK

SEQ ID NO: 175 - protein, 10 aa, Homo sapiens
IDDIWNLEVK

SEQ ID NO: 176 - protein, 11 aa, Homo sapiens
ENFAGEATLQR

SEQ ID NO: 177 - protein, 10 aa, Homo sapiens
IYSLWEHSTK

SEQ ID NO: 178 - protein, 18 aa, Homo sapiens
NHLQLEGLFFTNGEHTSK

SEQ ID NO: 179 - protein, 18 aa, Homo sapiens
IHSGSFQSQVELSNDQEK

SEQ ID NO: 180 - protein, 14 aa, Homo sapiens
AHLDIAGSLEGHLR

SEQ ID NO: 181 - protein, 9 aa, Homo sapiens
NIILPVYDK

SEQ ID NO: 182 - protein, 7 aa, Homo sapiens
SLWDFLK

SEQ ID NO: 183 - protein, 9 aa, Homo sapiens
LDVTTSIGR

SEQ ID NO: 184 - protein, 9 aa, Homo sapiens
VSTAFVYTK

SEQ ID NO: 185 - protein, 12 aa, Homo sapiens
NPNGYSFSIPVK

SEQ ID NO: 186 - protein, 7 aa, Homo sapiens
FIIPGLK

SEQ ID NO: 187 - protein, 6 aa, Homo sapiens
EIQIYK

SEQ ID NO: 188 - protein, 15 aa, Homo sapiens
TSSFALNLPTLPEVK

SEQ ID NO: 189 - protein, 17 aa, Homo sapiens
SVSDGIAALDLNAVANK

SEQ ID NO: 190 - protein, 22 aa, Homo sapiens
IADFELPTIIVPEQTIEIPSIK

SEQ ID NO: 191 - protein, 18 aa, Homo sapiens
FSVPAGIVIPSFQALTAR

SEQ ID NO: 192 - protein, 17 aa, Homo sapiens
FEVDSPVYNATWSASLK

SEQ ID NO: 193 - protein, 9 aa, Homo sapiens
IEDGTLASK

SEQ ID NO: 194 - protein, 6 aa, Homo sapiens
GTFAHR

SEQ ID NO: 195 - protein, 11 aa, Homo sapiens
DFSAEYEEDGK

SEQ ID NO: 196 - protein, 10 aa, Homo sapiens
YEGLQEWEGK

SEQ ID NO: 197 - protein, 6 aa, Homo sapiens
AHLNIK

SEQ ID NO: 198 - protein, 10 aa, Homo sapiens
SPAFTDLHLR

SEQ ID NO: 199 - protein, 13 aa, Homo sapiens
WNFYYSPQSSPDK

SEQ ID NO: 200 - protein, 9 aa, Homo sapiens
ESDEETQIK

SEQ ID NO: 201 - protein, 16 aa, Homo sapiens
VNWEEEAASGLLTSLK

SEQ ID NO: 202 - protein, 11 aa, Homo sapiens
ATGVLYDYVNK

SEQ ID NO: 203 - protein, 11 aa, Homo sapiens
YHWEHTGLTLR

SEQ ID NO: 204 - protein, 15 aa, Homo sapiens
NLQNNAEWVYQGAIR

SEQ ID NO: 205 - protein, 8 aa, Homo sapiens
QIDDIDVR

SEQ ID NO: 206 - protein, 13 aa, Homo sapiens
AASGTTGTYQEWK

SEQ ID NO: 207 - protein, 21 aa, Homo sapiens
AQNLYQELLTQEGQASFQGLK

SEQ ID NO: 208 - protein, 9 aa, Homo sapiens
DNVFDGLVR

SEQ ID NO: 209 - protein, 14 aa, Homo sapiens
HLIDSLIDFLNFPR

SEQ ID NO: 210 - protein, 6 aa, Homo sapiens
FQFPGK

SEQ ID NO: 211 - protein, 12 aa, Homo sapiens
EVGTVLSQVYSK

SEQ ID NO: 212 - protein, 24 aa, Homo sapiens
VHNGSEILFSYFQDLVITLPFELR

SEQ ID NO: 213 - protein, 7 aa, Homo sapiens
EAQEVFK

SEQ ID NO: 214 - protein, 18 aa, Homo sapiens
NLQDLLQFIFQLIEDNIK

SEQ ID NO: 215 - protein, 25 aa, Homo sapiens
FTYLINYIQDEINTIFSDYIPYVFK

SEQ ID NO: 216 - protein, 14 aa, Homo sapiens
EEYFDPSIVGWTVK

SEQ ID NO: 217 - protein, 7 aa, Homo sapiens
YYELEEK

SEQ ID NO: 218 - protein, 6 aa, Homo sapiens
IVSLIK

SEQ ID NO: 219 - protein, 7 aa, Homo sapiens
NLLVALK

SEQ ID NO: 220 - protein, 15 aa, Homo sapiens
NIQEYLSILTDPDGK

SEQ ID NO: 221 - protein, 13 aa, Homo sapiens
IAELSATAQEIIK

SEQ ID NO: 222 - protein, 7 aa, Homo sapiens
SQAIATK

SEQ ID NO: 223 - protein, 10 aa, Homo sapiens
IISDYHQQFR

SEQ ID NO: 224 - protein, 14 aa, Homo sapiens
LQDFSDQLSDYYEK

SEQ ID NO: 225 - protein, 6 aa, Homo sapiens
FIAESK

SEQ ID NO: 226 - protein, 21 aa, Homo sapiens
LIDLSIQNYHTFLIYITELLK

SEQ ID NO: 227 - protein, 24 aa, Homo sapiens
LELELRPTGEIEQYSVSATYELQR

SEQ ID NO: 228 - protein, 16 aa, Homo sapiens
DAVEKPQEFTIVAFVK

SEQ ID NO: 229 - protein, 20 aa, Homo sapiens
DKDQEVLLQTFLDDASPGDK

SEQ ID NO: 230 - protein, 12 aa, Homo sapiens
FQFPGKPGIYTR

SEQ ID NO: 231 - protein, 8 aa, Homo sapiens
VIDEEWQR

SEQ ID NO: 232 - protein, 19 aa, Homo sapiens
QLFEISVPLTSVPELVPVK

SEQ ID NO: 233 - protein, 7 aa, Homo sapiens
HPYSIIR

SEQ ID NO: 234 - protein, 15 aa, Homo sapiens
GDIGETGVPGAEGPR

SEQ ID NO: 235 - protein, 8 aa, Homo sapiens
GFPGIQGR

SEQ ID NO: 236 - protein, 11 aa, Homo sapiens
GEPGEGAYVYR

SEQ ID NO: 237 - protein, 15 aa, Homo sapiens
IFYNQQNHYDGSTGK

SEQ ID NO: 238 - protein, 6 aa, Homo sapiens
GEFVWK

SEQ ID NO: 239 - protein, 6 aa, Homo sapiens
ELISER

SEQ ID NO: 240 - protein, 10 aa, Homo sapiens
QGHNSVFLIK

SEQ ID NO: 241 - protein, 8 aa, Homo sapiens
VWVYPPEK

SEQ ID NO: 242 - protein, 6 aa, Homo sapiens
GEVPPR

SEQ ID NO: 243 - protein, 15 aa, Homo sapiens
LYLVQGTQVYVFLTK

SEQ ID NO: 244 - protein, 11 aa, Homo sapiens
GGYTLVSGYPK

SEQ ID NO: 245 - protein, 7 aa, Homo sapiens
LWWLDLK

SEQ ID NO: 246 - protein, 16 aa, Homo sapiens
SGAQATWTELPWPHEK

SEQ ID NO: 247 - protein, 24 aa, Homo sapiens
TQEPTQQHFSVAQVFLNNYDAENK

SEQ ID NO: 248 - protein, 8 aa, Homo sapiens
LFPDFFTR

SEQ ID NO: 249 - protein, 9 aa, Homo sapiens
VALYVDWIR

SEQ ID NO: 250 - protein, 12 aa, Homo sapiens
AQALEISGGFDK

SEQ ID NO: 251 - protein, 7 aa, Homo sapiens
STVEIFK

SEQ ID NO: 252 - protein, 6 aa, Homo sapiens
FEEQQR

SEQ ID NO: 253 - protein, 6 aa, Homo sapiens
SLVYLK

SEQ ID NO: 254 - protein, 17 aa, Homo sapiens
SVNDIVVLGPEQFYATR

SEQ ID NO: 255 - protein, 10 aa, Homo sapiens
WTYVLFYSPR

SEQ ID NO: 256 - protein, 10 aa, Homo sapiens
YVYVADVAAK

SEQ ID NO: 257 - protein, 10 aa, Homo sapiens
HDNWDLTQLK

SEQ ID NO: 258 - protein, 16 aa, Homo sapiens
LLNYNPEDPPGSEVLR

SEQ ID NO: 259 - protein, 24 aa, Homo sapiens
VSTVYANNGSVLQGTSVASVYHGK

SEQ ID NO: 260 - protein, 10 aa, Homo sapiens
IQNVLSEKPR

SEQ ID NO: 261 - protein, 7 aa, Homo sapiens
EYEPYFK

SEQ ID NO: 262 - protein, 16 aa, Homo sapiens
LSEGASYLDHTFPAEK

SEQ ID NO: 263 - protein, 10 aa, Homo sapiens
GTLTEGGTQK

SEQ ID NO: 264 - protein, 13 aa, Homo sapiens
QIVLLFAVFDESK

SEQ ID NO: 265 - protein, 9 aa, Homo sapiens
WIISSLTPK

SEQ ID NO: 266 - protein, 13 aa, Homo sapiens
SQHLDNFSNQIGK

SEQ ID NO: 267 - protein, 10 aa, Homo sapiens
EDGILGPIIR

SEQ ID NO: 268 - protein, 11 aa, Homo sapiens
AVQPGETYTYK

SEQ ID NO: 269 - protein, 17 aa, Homo sapiens
AADIEQQAVFAVFDENK

SEQ ID NO: 270 - protein, 10 aa, Homo sapiens
SWYLEDNINK

SEQ ID NO: 271 - protein, 17 aa, Homo sapiens
LEPEDEESDADYDYQNR

SEQ ID NO: 272 - protein, 8 aa, Homo sapiens
LAAALGIR

SEQ ID NO: 273 - protein, 11 aa, Homo sapiens
FTVNNLAEPQK

SEQ ID NO: 274 - protein, 15 aa, Homo sapiens
APSHQQATTAGSPLR

SEQ ID NO: 275 - protein, 10 aa, Homo sapiens
LLSLGAGEFK

SEQ ID NO: 276 - protein, 6 aa, Homo sapiens
SQEHAK

SEQ ID NO: 277 - protein, 15 aa, Homo sapiens
PWEDLPSQDTGSPSR

SEQ ID NO: 278 - protein, 10 aa, Homo sapiens
DPPSDLLLLK

SEQ ID NO: 279 - protein, 6 aa, Homo sapiens
QSNSSK

SEQ ID NO: 280 - protein, 7 aa, Homo sapiens
WHLASEK

SEQ ID NO: 281 - protein, 11 aa, Homo sapiens
AWGESTPLANK

SEQ ID NO: 282 - protein, 6 aa, Homo sapiens
QSGHPK

SEQ ID NO: 283 - protein, 6 aa, Homo sapiens
SQFLIK

SEQ ID NO: 284 - protein, 10 aa, Homo sapiens
HTHHAPLSPR

SEQ ID NO: 285 - protein, 6 aa, Homo sapiens
TFHPLR

SEQ ID NO: 286 - protein, 10 aa, Homo sapiens
SEAYNTFSER

SEQ ID NO: 287 - protein, 7 aa, Homo sapiens
HSLVLHK

SEQ ID NO: 288 - protein, 11 aa, Homo sapiens
DGTDYIEIIPK

SEQ ID NO: 289 - protein, 12 aa, Homo sapiens
EFNPLVIVGLSK

SEQ ID NO: 290 - protein, 23 aa, Homo sapiens
EEVQSSEDDYAEIDYVPYDDPYK

SEQ ID NO: 291 - protein, 7 aa, Homo sapiens
TNINSSR

SEQ ID NO: 292 - protein, 11 aa, Homo sapiens
DPDNIAAWYLR

SEQ ID NO: 293 - protein, 6 aa, Homo sapiens
SNNGNR

SEQ ID NO: 294 - protein, 19 aa, Homo sapiens
NYYIAAEEISWDYSEFVQR

SEQ ID NO: 295 - protein, 17 aa, Homo sapiens
ETDIEDSDDIPEDTTYK

SEQ ID NO: 296 - protein, 8 aa, Homo sapiens
YLDSTFTK

SEQ ID NO: 297 - protein, 15 aa, Homo sapiens
GEYEEHLGILGPIIR

SEQ ID NO: 298 - protein, 13 aa, Homo sapiens
PYSLHAHGLSYEK

SEQ ID NO: 299 - protein, 11 aa, Homo sapiens
TYEDDSPEWFK

SEQ ID NO: 300 - protein, 20 aa, Homo sapiens
EDNAVQPNSSYTYVWHATER

SEQ ID NO: 301 - protein, 12 aa, Homo sapiens
AWAYYSAVNPEK

SEQ ID NO: 302 - protein, 6 aa, Homo sapiens
SWYYEK

SEQ ID NO: 303 - protein, 15 aa, Homo sapiens
QHQLGVWPLLPGSFK

SEQ ID NO: 304 - protein, 11 aa, Homo sapiens
ASEFLGYWEPR

SEQ ID NO: 305 - protein, 14 aa, Homo sapiens
LNNGGSYNAWSVEK

SEQ ID NO: 306 - protein, 13 aa, Homo sapiens
EVIITGIQTQGAK

SEQ ID NO: 307 - protein, 11 aa, Homo sapiens
ENQFDPPIVAR

SEQ ID NO: 308 - protein, 8 aa, Homo sapiens
QITASSFK

SEQ ID NO: 309 - protein, 11 aa, Homo sapiens
SWWGDYWEPFR

SEQ ID NO: 310 - protein, 7 aa, Homo sapiens
VNAWQAK

SEQ ID NO: 311 - protein, 9 aa, Homo sapiens
QWLEIDLLK

SEQ ID NO: 312 - protein, 14 aa, Homo sapiens
SYTIHYSEQGVEWK

SEQ ID NO: 313 - protein, 9 aa, Homo sapiens
IFEGNTNTK

SEQ ID NO: 314 - protein, 10 aa, Homo sapiens
NFFNPPIISR

SEQ ID NO: 315 - protein, 9 aa, Homo sapiens
TWNQSIALR

SEQ ID NO: 316 - protein, 18 aa, Homo sapiens
ASKPGWWLLNTEVGENQR

SEQ ID NO: 317 - protein, 8 aa, Homo sapiens
AYNRPTLR

SEQ ID NO: 318 - protein, 24 aa, Homo sapiens
EKPQSTISGLLGPTLYAEVGDIIK

SEQ ID NO: 319 - protein, 13 aa, Homo sapiens
ADKPLSIHPQGIR

SEQ ID NO: 320 - protein, 14 aa, Homo sapiens
AWGESTPLANKPGK

SEQ ID NO: 321 - protein, 18 aa, Homo sapiens
NLASRPYSLHAHGLSYEK

SEQ ID NO: 322 - protein, 9 aa, Homo sapiens
GYTLADNGK

SEQ ID NO: 323 - protein, 19 aa, Homo sapiens
NTEQEEGGEAVHEVEVVIK

SEQ ID NO: 324 - protein, 11 aa, Homo sapiens
ETYDFDIAVLR

SEQ ID NO: 325 - protein, 6 aa, Homo sapiens
TPITFR

SEQ ID NO: 326 - protein, 13 aa, Homo sapiens
SHAPEVITSSPLK

SEQ ID NO: 327 - protein, 15 aa, Homo sapiens
VEDLHVGATVAPSSR

SEQ ID NO: 328 - protein, 8 aa, Homo sapiens
DFTFDLYR

SEQ ID NO: 329 - protein, 13 aa, Homo sapiens
GFQQLLQELNQPR

SEQ ID NO: 330 - protein, 13 aa, Homo sapiens
TLYLADTFPTNFR

SEQ ID NO: 331 - protein, 8 aa, Homo sapiens
QINDYVAK

SEQ ID NO: 332 - protein, 8 aa, Homo sapiens
WETSFNHK

SEQ ID NO: 333 - protein, 16 aa, Homo sapiens
GTQEQDFYVTSETVVR

SEQ ID NO: 334 - protein, 10 aa, Homo sapiens
EDQYHYLLDR

SEQ ID NO: 335 - protein, 21 aa, Homo sapiens
VVGVPYQGNATALFILPSEGK

SEQ ID NO: 336 - protein, 8 aa, Homo sapiens
QLELYLPK

SEQ ID NO: 337 - protein, 11 aa, Homo sapiens
FSIEGSYQLEK

SEQ ID NO: 338 - protein, 11 aa, Homo sapiens
AVVEVDESGTR

SEQ ID NO: 339 - protein, 8 aa, Homo sapiens
GPLDQLEK

SEQ ID NO: 340 - protein, 18 aa, Homo sapiens
GGETAQSADPQWEQLNNK

SEQ ID NO: 341 - protein, 23 aa, Homo sapiens
ENTVTNDWIPEGEEDDDYLDLEK

SEQ ID NO: 342 - protein, 7 aa, Homo sapiens
LNILNAK

SEQ ID NO: 343 - protein, 14 aa, Homo sapiens
GETHEQVHSILHFK

SEQ ID NO: 344 - protein, 8 aa, Homo sapiens
DFVNASSK

SEQ ID NO: 345 - protein, 11 aa, Homo sapiens
YEITTIHNLFR

SEQ ID NO: 346 - protein, 7 aa, Homo sapiens
NFGYTLR

SEQ ID NO: 347 - protein, 9 aa, Homo sapiens
SVNDLYIQK

SEQ ID NO: 348 - protein, 9 aa, Homo sapiens
QFPILLDFK

SEQ ID NO: 349 - protein, 20 aa, Homo sapiens
EYYFAEAQIADFSDPAFISK

SEQ ID NO: 350 - protein, 6 aa, Homo sapiens
GSWVNK

SEQ ID NO: 351 - protein, 9 aa, Homo sapiens
NYNLVESLK

SEQ ID NO: 352 - protein, 7 aa, Homo sapiens
IAIDLFK

SEQ ID NO: 353 - protein, 10 aa, Homo sapiens
PFLFLIYEHR

SEQ ID NO: 354 - protein, 6 aa, Homo sapiens
VANPSR

SEQ ID NO: 355 - protein, 15 aa, Homo sapiens
FTVDRPFLFLIYEHR

SEQ ID NO: 356 - protein, 12 aa, Homo sapiens
SFSIIGDFQNGK

SEQ ID NO: 357 - protein, 12 aa, Homo sapiens
GLYLETEAGYYK

SEQ ID NO: 358 - protein, 11 aa, Homo sapiens
LSGEAYGFVAR

SEQ ID NO: 359 - protein, 14 aa, Homo sapiens
IDGSGNFQVLLSDR

SEQ ID NO: 360 - protein, 14 aa, Homo sapiens
DHSGQVFSVVSNGK

SEQ ID NO: 361 - protein, 13 aa, Homo sapiens
AGTLDLSLTVQGK

SEQ ID NO: 362 - protein, 17 aa, Homo sapiens
QHVVSVEEALLATGQWK

SEQ ID NO: 363 - protein, 10 aa, Homo sapiens
SITLFVQEDR

SEQ ID NO: 364 - protein, 7 aa, Homo sapiens
DLASIAR

SEQ ID NO: 365 - protein, 15 aa, Homo sapiens
GGVNDNFQGVLQNVR

SEQ ID NO: 366 - protein, 12 aa, Homo sapiens
FVFGTTPEDILR

SEQ ID NO: 367 - protein, 7 aa, Homo sapiens
TNYIGHK

SEQ ID NO: 368 - protein, 11 aa, Homo sapiens
TIVTTLQDSIR

SEQ ID NO: 369 - protein, 7 aa, Homo sapiens
ELANELR

SEQ ID NO: 370 - protein, 18 aa, Homo sapiens
FTGSQPFGQGVEHATANK

SEQ ID NO: 371 - protein, 6 aa, Homo sapiens
DDVGDR

SEQ ID NO: 372 - protein, 8 aa, Homo sapiens
LVPNPDQK

SEQ ID NO: 373 - protein, 12 aa, Homo sapiens
GTSQNDPNWVVR

SEQ ID NO: 374 - protein, 13 aa, Homo sapiens
QVTQSYWDTNPTR

SEQ ID NO: 375 - protein, 10 aa, Homo sapiens
AQGYSGLSVK

SEQ ID NO: 376 - protein, 13 aa, Homo sapiens
VVNSTTGPGEHLR

SEQ ID NO: 377 - protein, 6 aa, Homo sapiens
DFTAYR

SEQ ID NO: 378 - protein, 6 aa, Homo sapiens
TEIAER

SEQ ID NO: 379 - protein, 9 aa, Homo sapiens
VVLGDQDLK

SEQ ID NO: 380 - protein, 9 aa, Homo sapiens
EEFHEQSFR

SEQ ID NO: 381 - protein, 7 aa, Homo sapiens
YSHYNER

SEQ ID NO: 382 - protein, 9 aa, Homo sapiens
PGVYTQVTK

SEQ ID NO: 383 - protein, 6 aa, Homo sapiens
FLNWIK

SEQ ID NO: 384 - protein, 10 aa, Homo sapiens
RPGVYTQVTK

SEQ ID NO: 385 - protein, 11 aa, Homo sapiens
LEEFVQGNLER

SEQ ID NO: 386 - protein, 8 aa, Homo sapiens
EVFENTER

SEQ ID NO: 387 - protein, 6 aa, Homo sapiens
TTEFWK

SEQ ID NO: 388 - protein, 6 aa, Homo sapiens
NSADNK

SEQ ID NO: 389 - protein, 6 aa, Homo sapiens
LAENQK

SEQ ID NO: 390 - protein, 8 aa, Homo sapiens
VSVSQTSK

SEQ ID NO: 391 - protein, 8 aa, Homo sapiens
VVGGEDAK

SEQ ID NO: 392 - protein, 13 aa, Homo sapiens
PGQFPWQVVLNGK

SEQ ID NO: 393 - protein, 19 aa, Homo sapiens
ITVVAGEHNIEETEHTEQK

SEQ ID NO: 394 - protein, 13 aa, Homo sapiens
IIPHHNYNAAINK

SEQ ID NO: 395 - protein, 8 aa, Homo sapiens
EYTNIFLK

SEQ ID NO: 396 - protein, 11 aa, Homo sapiens
FGSGYVSGWGR

SEQ ID NO: 397 - protein, 6 aa, Homo sapiens
VPLVDR

SEQ ID NO: 398 - protein, 9 aa, Homo sapiens
SLGVGFATR

SEQ ID NO: 399 - protein, 8 aa, Homo sapiens
NGDILTLK

SEQ ID NO: 400 - protein, 6 aa, Homo sapiens
THSTFK

SEQ ID NO: 401 - protein, 13 aa, Homo sapiens
LGVEFDETTADDR

SEQ ID NO: 402 - protein, 9 aa, Homo sapiens
SIVTLDGGK

SEQ ID NO: 403 - protein, 11 aa, Homo sapiens
WDGQETTLVRV

SEQ ID NO: 404 - protein, 13 aa, Homo sapiens
VGPAVELALAQVK

SEQ ID NO: 405 - protein, 10 aa, Homo sapiens
PDLLPGWTVR

SEQ ID NO: 406 - protein, 6 aa, Homo sapiens
FTAHWR

SEQ ID NO: 407 - protein, 16 aa, Homo sapiens
VPLLTAGAPALGFGVK

SEQ ID NO: 408 - protein, 8 aa, Homo sapiens
DEYALTTR

SEQ ID NO: 409 - protein, 7 aa, Homo sapiens
AGPSYAK

SEQ ID NO: 410 - protein, 10 aa, Homo sapiens
LGDFVAALHR

SEQ ID NO: 411 - protein, 20 aa, Homo sapiens
LNITVDHLEFAEDDLSHYTR

SEQ ID NO: 412 - protein, 9 aa, Homo sapiens
GDGQDVSAR

SEQ ID NO: 413 - protein, 7 aa, Homo sapiens
QAFQAAK

SEQ ID NO: 414 - protein, 12 aa, Homo sapiens
DPDNPEYLEFLK

SEQ ID NO: 415 - protein, 10 aa, Homo sapiens
SFQGVTGYLK

SEQ ID NO: 416 - protein, 7 aa, Homo sapiens
IDSSGDR

SEQ ID NO: 417 - protein, 18 aa, Homo sapiens
VVLNYNGTSQELVAVSGR

SEQ ID NO: 418 - protein, 14 aa, Homo sapiens
LNWPLGYPPPDIPK

SEQ ID NO: 419 - protein, 8 aa, Homo sapiens
ELASELWR

SEQ ID NO: 420 - protein, 11 aa, Homo sapiens
WEDVEPSSLER

SEQ ID NO: 421 - protein, 19 aa, Homo sapiens
GSNYGSLLTTEGQFQVFAK

SEQ ID NO: 422 - protein, 7 aa, Homo sapiens
GNLVAVK

SEQ ID NO: 423 - protein, 9 aa, Homo sapiens
DVQNEHLTR

SEQ ID NO: 424 - protein, 9 aa, Homo sapiens
YSLTNDIVK

SEQ ID NO: 425 - protein, 13 aa, Homo sapiens
DLDPEQGHTVYAK

SEQ ID NO: 426 - protein, 20 aa, Homo sapiens
GSQAGDVYSFGIILQEIALR

SEQ ID NO: 427 - protein, 14 aa, Homo sapiens
SGVFHVEGLDLSPK

SEQ ID NO: 428 - protein, 7 aa, Homo sapiens
GEQPPFR

SEQ ID NO: 429 - protein, 7 aa, Homo sapiens
PPFQQIR

SEQ ID NO: 430 - protein, 13 aa, Homo sapiens
ENSSNILDNLLSR

SEQ ID NO: 431 - protein, 8 aa, Homo sapiens
IHLSSETK

SEQ ID NO: 432 - protein, 14 aa, Homo sapiens
AVLEEFGGFELELR

SEQ ID NO: 433 - protein, 8 aa, Homo sapiens
HRPQEQLR

SEQ ID NO: 434 - protein, 12 aa, Homo sapiens
ARPDLLPGWTVR

SEQ ID NO: 435 - protein, 16 aa, Homo sapiens
LTQLGTFEDHFLSLQR

SEQ ID NO: 436 - protein, 8 aa, Homo sapiens
NYDLSFLK

SEQ ID NO: 437 - protein, 18 aa, Homo sapiens
TIQEVAGYVLIALNTVER

SEQ ID NO: 438 - protein, 10 aa, Homo sapiens
IPLENLQIIR

SEQ ID NO: 439 - protein, 11 aa, Homo sapiens
NLQEILHGAVR

SEQ ID NO: 440 - protein, 11 aa, Homo sapiens
DSLSINATNIK

SEQ ID NO: 441 - protein, 19 aa, Homo sapiens
GDSFTHTPPLDPQELDILK

SEQ ID NO: 442 - protein, 11 aa, Homo sapiens
DFLSLIVSILR

SEQ ID NO: 443 - protein, 14 aa, Homo sapiens
SWNEPLYHLVTEVR

SEQ ID NO: 444 - protein, 9 aa, Homo sapiens
AVEIEEQTK

SEQ ID NO: 445 - protein, 6 aa, Homo sapiens
IDNYLK

SEQ ID NO: 446 - protein, 6 aa, Homo sapiens
TLPFSR

SEQ ID NO: 447 - protein, 12 aa, Homo sapiens
DNYTDLVAIQNK

SEQ ID NO: 448 - protein, 12 aa, Homo sapiens
IGGIWTWVGTNK

SEQ ID NO: 449 - protein, 17 aa, Homo sapiens
SLTEEAENWGDGEPNNK

SEQ ID NO: 450 - protein, 12 aa, Homo sapiens
NAGVEGSLIVEK

SEQ ID NO: 451 - protein, 12 aa, Homo sapiens
IDAVYEAPQEEK

SEQ ID NO: 452 - protein, 8 aa, Homo sapiens
IILLSYGK

SEQ ID NO: 453 - protein, 11 aa, Homo sapiens
IPAGATNVDIR

SEQ ID NO: 454 - protein, 7 aa, Homo sapiens
LYNPDVR

SEQ ID NO: 455 - protein, 11 aa, Homo sapiens
SPEDSSPDAAR

SEQ ID NO: 456 - protein, 15 aa, Homo sapiens
NWGLSVYADKPETTK

SEQ ID NO: 457 - protein, 9 aa, Homo sapiens
EIGELYLPK

SEQ ID NO: 458 - protein, 15 aa, Homo sapiens
LQHLENELTHDIITK

SEQ ID NO: 459 - protein, 10 aa, Homo sapiens
DLVFSTWDHK

SEQ ID NO: 460 - protein, 12 aa, Homo sapiens
ALQDQLVLVAAK

SEQ ID NO: 461 - protein, 13 aa, Homo sapiens
FATTFYQHLADSK

SEQ ID NO: 462 - protein, 9 aa, Homo sapiens
SPELQAEAK

SEQ ID NO: 463 - protein, 12 aa, Homo sapiens
SLAPYAQDTQEK

SEQ ID NO: 464 - protein, 9 aa, Homo sapiens
EFGNTLEDK

SEQ ID NO: 465 - protein, 9 aa, Homo sapiens
TYLPAVDEK

SEQ ID NO: 466 - protein, 11 aa, Homo sapiens
GWVTDGFSSLK

SEQ ID NO: 467 - protein, 7 aa, Homo sapiens
VLNQELR

SEQ ID NO: 468 - protein, 9 aa, Homo sapiens
LGPLVEQGR

SEQ ID NO: 469 - protein, 13 aa, Homo sapiens
WWTQAQAHDLVIK

SEQ ID NO: 470 - protein, 12 aa, Homo sapiens
TPENYPNAGLTR

SEQ ID NO: 471 - protein, 12 aa, Homo sapiens
ALSLASSSGSDK

SEQ ID NO: 472 - protein, 10 aa, Homo sapiens
LTIVHHPGGK

SEQ ID NO: 473 - protein, 10 aa, Homo sapiens
SAPAAAIAAR

SEQ ID NO: 474 - protein, 6 aa, Homo sapiens
AFVFPK

SEQ ID NO: 475 - protein, 16 aa, Homo sapiens
FHTFPQTAIGVGAPGK

SEQ ID NO: 476 - protein, 11 aa, Homo sapiens
NNFVPTNVGSK

SEQ ID NO: 477 - protein, 6 aa, Homo sapiens
VNVGVK

SEQ ID NO: 478 - protein, 8 aa, Homo sapiens
SQFEGFVK

SEQ ID NO: 479 - protein, 13 aa, Homo sapiens
GAYPLSIEPIGVR

SEQ ID NO: 480 - protein, 8 aa, Homo sapiens
PDLSFPVK

SEQ ID NO: 481 - protein, 11 aa, Homo sapiens
ELDESLQVAER

SEQ ID NO: 482 - protein, 23 aa, Homo sapiens
VAQVIIPSTYVPGTTNHDIALLR

SEQ ID NO: 483 - protein, 9 aa, Homo sapiens
LHPTHYSIR

SEQ ID NO: 484 - protein, 10 aa, Homo sapiens
TSESGLPSTR

SEQ ID NO: 485 - protein, 9 aa, Homo sapiens
VVGGLVALR

SEQ ID NO: 486 - protein, 25 aa, Homo sapiens
AVPPNNSNAAEDDLPTVELQGVVPR

SEQ ID NO: 487 - protein, 9 aa, Homo sapiens
IQTHSTTYR

SEQ ID NO: 488 - protein, 9 aa, Homo sapiens
GVVGLPGQR

SEQ ID NO: 489 - protein, 12 aa, Homo sapiens
GGAGPPGPEGGK

SEQ ID NO: 490 - protein, 9 aa, Homo sapiens
GVVGPQGAR

SEQ ID NO: 491 - protein, 9 aa, Homo sapiens
ITQVLHFTK

SEQ ID NO: 492 - protein, 10 aa, Homo sapiens
DLFDPIIEDR

SEQ ID NO: 493 - protein, 7 aa, Homo sapiens
FEEILTR

SEQ ID NO: 494 - protein, 10 aa, Homo sapiens
AELIVQPELK

SEQ ID NO: 495 - protein, 10 aa, Homo sapiens
GSHGLEIFQR

SEQ ID NO: 496 - protein, 10 aa, Homo sapiens
YTHLVAIQNK

SEQ ID NO: 497 - protein, 13 aa, Homo sapiens
LPGGYGLPYTTGK

SEQ ID NO: 498 - protein, 8 aa, Homo sapiens
LVSALHTR

SEQ ID NO: 499 - protein, 9 aa, Homo sapiens
TLAFPLTIR

SEQ ID NO: 500 - protein, 9 aa, Homo sapiens
ALENLLPTK

SEQ ID NO: 501 - protein, 10 aa, Homo sapiens
IPLENLQIIR

SEQ ID NO: 502 - protein, 9 aa, Homo sapiens
SLGVGFATR

SEQ ID NO: 503 - protein, 9 aa, Homo sapiens
VQHIQLLQK

SEQ ID NO: 504 - protein, 7 aa, Homo sapiens
YQISVNK

SEQ ID NO: 505 - protein, 13 aa, Homo sapiens
YEASILTHDSSIR

SEQ ID NO: 506 - protein, 14 aa, Homo sapiens
SYTITGLQPGTDYK

SEQ ID NO: 507 - protein, 10 aa, Homo sapiens
LGAEVYHTLK

SEQ ID NO: 508 - protein, 10 aa, Homo sapiens
LADVYQAELR

SEQ ID NO: 509 - protein, 8 aa, Homo sapiens
FSYIPEAK

SEQ ID NO: 510 - protein, 11 aa, Homo sapiens
EPGGPSFTIGK

SEQ ID NO: 511 - protein, 7 aa, Homo sapiens
TLGLYGK

SEQ ID NO: 512 - protein, 12 aa, Homo sapiens
DIAPTLTLYVGK

SEQ ID NO: 513 - protein, 10 aa, Homo sapiens
LFDQAFGLPR

SEQ ID NO: 514 - protein, 12 aa, Homo sapiens
DEIPHNDIALLK

SEQ ID NO: 515 - protein, 12 aa, Homo sapiens
GPSVFPLAPSSK

SEQ ID NO: 516 - protein, 15 aa, Homo sapiens
GFYFNKPTGYGSSSR

SEQ ID NO: 517 - protein, 14 aa, Homo sapiens
ALPGEQQPLHALTR

SEQ ID NO: 518 - protein, 14 aa, Homo sapiens
ASVSVTAEDEGTQR

SEQ ID NO: 519 - protein, 11 aa, Homo sapiens
AHVNSLGENLK

SEQ ID NO: 520 - protein, 9 aa, Homo sapiens
FESSEEQAR

SEQ ID NO: 521 - protein, 11 aa, Homo sapiens
VTGKPDAETLK

SEQ ID NO: 522 - protein, 11 aa, Homo sapiens
SLGPALLLLQK

SEQ ID NO: 523 - protein, 9 aa, Homo sapiens
FTEGPPLHK

SEQ ID NO: 524 - protein, 15 aa, Homo sapiens
LGLGADVAQVTGALR

SEQ ID NO: 525 - protein, 12 aa, Homo sapiens
GFQALGDAADIR

SEQ ID NO: 526 - protein, 7 aa, Homo sapiens
EYLIAGK

SEQ ID NO: 527 - protein, 13 aa, Homo sapiens
VVPASADPADTEK

SEQ ID NO: 528 - protein, 10 aa, Homo sapiens
EADLPEPSEK

SEQ ID NO: 529 - protein, 10 aa, Homo sapiens
TAHYGSLPQK

SEQ ID NO: 530 - protein, 7 aa, Homo sapiens
VFFASWR

SEQ ID NO: 531 - protein, 13 aa, Homo sapiens
IAQLEEQVEQEAR

SEQ ID NO: 532 - protein, 10 aa, Homo sapiens
TPEEYPESAK

SEQ ID NO: 533 - protein, 8 aa, Homo sapiens
EVATEGIR

SEQ ID NO: 534 - protein, 18 aa, Homo sapiens
VDAVFQQEHFFHVFSGPR

SEQ ID NO: 535 - protein, 6 aa, Homo sapiens
ITLYGR

SEQ ID NO: 536 - protein, 8 aa, Homo sapiens
PFFAGLVK

SEQ ID NO: 537 - protein, 11 aa, Homo sapiens
SLQSELDEINK

SEQ ID NO: 538 - protein, 8 aa, Homo sapiens
LEGQISAR

SEQ ID NO: 539 - protein, 8 aa, Homo sapiens
TWTWVGTK

SEQ ID NO: 540 - protein, 9 aa, Homo sapiens
AYLDVNELK

SEQ ID NO: 541 - protein, 8 aa, Homo sapiens
ADEGISFR

SEQ ID NO: 542 - protein, 10 aa, Homo sapiens
LLDSLPSDTR

SEQ ID NO: 543 - protein, 7 aa, Homo sapiens
LFLEPTR

SEQ ID NO: 544 - protein, 10 aa, Homo sapiens
VFQQVAQASK

SEQ ID NO: 545 - protein, 16 aa, Homo sapiens
VIAPVDEVQISILSSK

SEQ ID NO: 546 - protein, 8 aa, Homo sapiens
EGDPAPYK

SEQ ID NO: 547 - protein, 9 aa, Homo sapiens
ELLETGDNR

SEQ ID NO: 548 - protein, 7 aa, Homo sapiens
EVAAQVK

SEQ ID NO: 549 - protein, 7 aa, Homo sapiens
ELANTIK

SEQ ID NO: 550 - protein, 7 aa, Homo sapiens
EQEVVDK

SEQ ID NO: 551 - protein, 12 aa, Homo sapiens
ETAASLLQAGYK

SEQ ID NO: 552 - protein, 13 aa, Homo sapiens
ETQSTPQSAPQVR

SEQ ID NO: 553 - protein, 11 aa, Homo sapiens
IQEVAGSLIFR

SEQ ID NO: 554 - protein, 9 aa, Homo sapiens
EANYIGSDK

SEQ ID NO: 555 - protein, 8 aa, Homo sapiens
GNYDAAQR

SEQ ID NO: 556 - protein, 16 aa, Homo sapiens
IFFYDSENPPASEVLR

SEQ ID NO: 557 - protein, 9 aa, Homo sapiens
ILIGTVFHK

SEQ ID NO: 558 - protein, 8 aa, Homo sapiens
TSSSFEVR

SEQ ID NO: 559 - protein, 14 aa, Homo sapiens
YYSTTHGALLSGQR

SEQ ID NO: 560 - protein, 11 aa, Homo sapiens
ENVLDEVANAK

SEQ ID NO: 561 - protein, 8 aa, Homo sapiens
TYFFVEDK

SEQ ID NO: 562 - protein, 10 aa, Homo sapiens
IYLSDSLTGK

SEQ ID NO: 563 - protein, 9 aa, Homo sapiens
YLPGYYSEK

SEQ ID NO: 564 - protein, 8 aa, Homo sapiens
EVVLQHVR

SEQ ID NO: 565 - protein, 9 aa, Homo sapiens
FQDLVDAVR

SEQ ID NO: 566 - protein, 12 aa, Homo sapiens
FVFGTTPEDILR

SEQ ID NO: 567 - protein, 9 aa, Homo sapiens
FYYNSVIGK

SEQ ID NO: 568 - protein, 9 aa, Homo sapiens
VVPGEEEQK

SEQ ID NO: 569 - protein, 20 aa, Homo sapiens
AAQSGDYGGAGDDYVLGSLR

SEQ ID NO: 570 - protein, 10 aa, Homo sapiens
LGLSDHEIDR

SEQ ID NO: 571 - protein, 8 aa, Homo sapiens
DEQVPFSK

SEQ ID NO: 572 - protein, 8 aa, Homo sapiens
AVLENALR

SEQ ID NO: 573 - protein, 11 aa, Homo sapiens
NTVISVNPSTK

SEQ ID NO: 574 - protein, 10 aa, Homo sapiens
VVSWIDVYTR

SEQ ID NO: 575 - protein, 7 aa, Homo sapiens
DLIQHPK

SEQ ID NO: 576 - protein, 7 aa, Homo sapiens
TETPVSK

SEQ ID NO: 577 - protein, 8 aa, Homo sapiens
TFVLNFIK

SEQ ID NO: 578 - protein, 8 aa, Homo sapiens
VYFAGFPR

SEQ ID NO: 579 - protein, 8 aa, Homo sapiens
GLLSGWAR

(19) United States
(12) Patent Application Publication
(10) Pub. No.: US 2018/0267020 A1
(43) Pub. Date: Sep. 20, 2018

(54) PANEL OF ACVS-ASSOCIATED PROTEINS FOR DIAGNOSIS AND PROGNOSIS

(71) Applicants: UVic Industry Partnerships Inc., Victoria (CA); Vancouver Island Health Authority, Victoria (CA); British Columbia Center for Disease Control, Vancouver (CA); The Governors of the University of Calgary, Calgary (CA)

(72) Inventors: Andrew Penn, Victoria (CA); Christoph Hermann Borchers, Victoria (CA); Mary Louise Lesperance, Victoria (CA); Angela Marie Jackson, Victoria (CA); Maximilian Barnaby Bibok, Erickson (CA); Viera Saly, Courtenay (CA); Robert Fred Balshaw, Winnipeg (CA); Shelagh Brown Coutts, Calgary (CA)

(73) Assignees: UVic Industry Partnerships Inc., Victoria (CA); Vancouver Island Health Authority, Victoria (CA); British Columbia Center for Disease Control, Vancouver (CA); The Governors of the University of Calgary, Calgary (CA)

(21) Appl. No.: 15/925,629

(22) Filed: Mar. 19, 2018

Related U.S. Application Data
(60) Provisional application No. 62/506,392, filed on May 15, 2017.
(60) Provisional application No. 62/473,214, filed on Mar. 17, 2017.

(51) Int. Cl. G01N 33/50; G01N 33/68

(52) U.S. Cl. CPC G01N 33/5023; G01N 33/6851; G01N 33/6842; G01N 2800/60; G01N 33/5091; G01N 33/6854; G01N 2800/2871; G01N 33/5044

(57) ABSTRACT

Provided herein are methods of diagnosing and/or treating ACVS, by determining expression levels of several ACVS-related molecules, such as FABP3, ANPR-1, IGFBP-3, F9, SELL, apoB100, ADPN, vWF, THBS1, PRL, PON3, EGFR, VEGF-D, HPX, MBT, F5, F10, SERPIN A5, HCII, and HABP2, for example in a blood sample.

### CROSS REFERENCE TO RELATED APPLICATIONS

This application claims priority to U.S. Provisional Application No. 62/506,392, filed May 15, 2017, and U.S. Provisional Application No. 62/473,214, filed Mar. 17, 2017. Both of these applications are incorporated herein by reference in their entirety.

### FIELD

This application provides methods of diagnosing and/or treating acute cerebrovascular syndrome (ACVS), which can include determining expression levels of ACVS-related molecules such as FABP3, ANPR-1, IGFBP-3, F9, SELL, apoB100, adiponectin, vWF, THBS1, PRL, PON3, EGFR, VEGF-D, HPX, myeloblastin, F5, F10, SERPIN A5, HCII, and HABP2.

### PARTIES TO JOINT RESEARCH AGREEMENT

This application describes and claims certain subject matter that was developed under a written joint research agreement between UVic Industry Partnerships Inc. and Vancouver Island Health Authority and an agreement between Vancouver Island Health Authority and the Governors of the University of Calgary.

### BACKGROUND

In the management of acute cerebrovascular syndrome (ACVS), the high prevalence of conditions that mimic stroke present a challenge, particularly for first-line physicians. Such mimics include migraine, Todd's paresis following seizure, delirium, compressive neuropathies, and many other entities. This is especially true with transient ischemic attack (TIA), where typically half of referrals to urgent specialty services are mimics.

Unlike cardiology, where an ECG and single blood test allows for effective filtering, the first step with ACVS may be advanced imaging and or specialist referral. The development and validation of a reliable blood biomarker test capable of distinguishing ACVS from mimic is challenging, despite numerous multi-center studies of varying size. Additionally, most stroke biomarker studies use ELISA technology, except certain studies that use newer methods for protein quantification, such as mass spectrometry. ELISA is an immunoassay that measures protein expression, but each protein requires a separate assay, even when a few are bundled together in a composite test. In contrast, mass spectrometry allows simultaneous quantitation of large numbers of biomarkers, in a rapid, reproducible, and sensitive assay at a low cost per sample. To date, no protein biomarkers have been successfully adopted into clinical practice; although, commercial ELISA kits for stroke are available.

Further, plasma protein levels fluctuate significantly in the general population due to heritable factors, individual and common environmental factors, and as yet unknown factors. For example, TIA and severe stroke lie on a continuum under the umbrella of ACVS, and the biological mechanisms dominating protein expression are unclear, particularly as TIA likely involves transcription from a more intact brain. Moreover, many potential stroke protein markers are low-abundance proteins (i.e., not easily or readily quantifiable), and their variability in the general population is not well-known. However, validating large protein panels requires many patients. Mass spectrometry is a cost effective tool for this task, as performance is currently inadequate.

Accordingly, a mass spectrometry assay with multiple proteins rather than a single “troponin” would be a desirable diagnostic blood test for ACVS.

### SUMMARY

Provided herein is a large-scale, multi-site, precision-medicine method, Spectrometry for TIA Rapid Assessment (SpecTRA), wherein mass spectrometry was used to measure 141 proteins concurrently in a clinical research program involving 1860 patients to verify and validate a clinically useful blood test for TIA and minor stroke. The natural abundance and variability of candidate plasma protein levels were examined in ischemic stroke patients and stroke-mimic patients to generate a protein biomarker panel. Severe stroke provides a robust target for up-regulated or down-regulated proteins.

A panel of ACVS-related biomarkers was identified, which in some examples, includes FABP3, ANPR-1, IGFBP-3, F9, SELL, apoB100, ADPN, vWF, THBS1, PRL, PON3, EGFR, VEGF-D, HPX, MBT, F5, F10, SERPIN A5, HCII, and HABP2. The expression of such markers can be detected in a sample, such as a blood sample from a mammalian subject, such as a human.

Methods are provided for treating a subject with acute cerebrovascular syndrome (ACVS). Such methods can include measuring at least two ACVS-related peptides derived from proteins in a sample obtained from a subject, including the ACVS-related proteins FABP3, ANPR-1, IGFBP-3, F9, SELL, apoB100, ADPN, vWF, THBS1, PRL, PON3, EGFR, VEGF-D, HPX, MBT, F5, F10, SERPIN A5, HCII, and HABP2. The methods can further include measuring differential expression of the ACVS-related proteins compared to a control representing expression for each of the ACVS-related proteins expected in a sample from a subject who does not have ACVS. In addition, the methods can include administering at least one of thrombolytic therapy, antiplatelet therapy, anticoagulant therapy, or surgery to the subject with ACVS, thereby treating the subject.

In some examples, the subject with ACVS has transient ischemic attack (TIA), and the ACVS-related peptides are TIA-related peptides.

In further examples, the methods include measuring the ACVS-related proteins FABP3, ANPR-1, IGFBP-3, F9, SELL, and apoB100. In additional examples, the methods include measuring FABP3, ANPR-1, IGFBP-3, F9, SELL, and apoB100, in addition to at least one of (such as 1, 2, 3, 4, 5, or 6 of) adiponectin, vWF, THBS1, PRL, PON3, EGFR, and VEGF-D.

In some examples, the methods include measuring the ACVS-related proteins IGFBP-3, F9, SELL, apoB100, ADPN, vWF, THBS1, PON3, VEGF-D, HPX, MBT, F5, F10, SERPIN A5, HCII, and HABP2. In other examples, the methods include measuring the ACVS-related proteins IGFBP-3, F9, SELL, apoB100, ADPN, vWF, PON3, VEGF-D, HPX, MBT, F5, F10, SERPIN A5, HCII, and HABP2. In still further examples, the methods include measuring the ACVS-related proteins IGFBP-3, F9, SELL, apoB100, and vWF, or the methods include measuring the ACVS-related proteins IGFBP-3, F9, SELL, and apoB100.

In certain examples of the methods, the presence of motor weakness, aphasia, and/or dysarthria in the subject is unknown and/or is not considered prior to performing the method. In other examples, motor weakness, aphasia, and/or dysarthria is not present in the subject. In some examples, the method further includes considering, measuring, or determining whether motor weakness, aphasia, and/or dysarthria is present in the subject.

Further, the methods can include measuring expression using a mass spectrometry assay. In some examples, the mass spectrometry assay can be an immuno matrix-assisted laser desorption/ionization (iMALDI) assay or Stable Isotope Standards and Capture by Anti-Peptide Antibodies (SISCAPA) assay. The iMALDI or SISCAPA assay can be used with polyclonal or monoclonal antibodies. In addition, the mass spectrometry assay can also include a multiple reaction monitoring (MRM) assay, a parallel reaction monitoring (PRM)-based targeted mass spectrometry or a basic MALDI assay. In certain other examples, the MRM assay can be an enriched MRM assay.

The methods can also include deriving ACVS-related peptides from proteins by using a protease. Such proteases can include at least one of an endoprotease, a nonspecific protease, trypsin, chymotryptsin, endoprotease Glu-C, endoprotese Lys-C, endoprotease AspN, endoprotease ArgC, elastinase, thermolysis, or pepsin. Further, in some examples of the methods, the ACVS-related peptides include the peptides listed in FIG. 5. In specific examples of measuring expression of ACVS-related proteins, measuring expression of IGFBP3 includes detecting SEQ ID NO: 1, measuring expression of SELL includes detecting SEQ ID NO: 2, measuring expression of apoB100 includes detecting SEQ ID NO: 3 and/or SEQ ID NO: 17, measuring expression of VEGF-D includes detecting SEQ ID NO: 4, measuring expression of ADPN includes detecting SEQ ID NO: 5, measuring expression of HPX includes detecting SEQ ID NO: 6; measuring expression of MBT includes detecting SEQ ID NO: 7, measuring expression of PON3 includes detecting SEQ ID NO: 8, measuring expression of F5 includes detecting SEQ ID NO: 9, measuring expression of F10 includes detecting SEQ ID NO: 10, measuring expression of SERPINA5 includes detecting SEQ ID NO: 11, measuring expression of HCF2 includes detecting SEQ ID NO: 12, measuring expression of vWF includes detecting SEQ ID NO: 13, measuring expression of THBS1 includes detecting SEQ ID NO: 14, measuring expression of HABP2 includes detecting SEQ ID NO: 15, measuring expression of F9 includes detecting SEQ ID NO: 16, measuring expression of FABP3 includes detecting SEQ ID NO: 18, and/or measuring expression of ANPR-1 includes detecting SEQ ID NO: 19.

The methods can include measuring expression using a multiplex assay or an individual assay for each ACVS-related protein or ACVS-related peptide. In some examples, the sample analyzed can be a blood sample, such as a whole blood sample, plasma, serum, and/or dried blood spots.

The foregoing and other objects and features of the disclosure will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Enriched MRM versus ELISA for the quantitation of S100A12—Scatterplot of enriched MRM measurement (log 2 Relative Area) of S100A12 concentration versus the corresponding ELISA-based concentration measurements (log 2 Abundance). The Pearson sample correlation is r=0.82. Mimics=blue, Stroke=red. Etiologies are marked with circle=Cardioembolism, square=Cyrptogenic, diamond=Large artery atherosclerosis, and triangle=Other.

FIG. 2. The first two principal components of the 31 statistically significant proteins clearly separate strokes and stroke-mimics. Mimics=blue; Stroke=red. Etiologies are marked with a circle (cardioembolism), square (cyrptogenic), diamond (large artery atherosclerosis), and triangle (other).

FIG. 3. Receiver operating characteristic (ROC) plot adjusted by cross-validation comparing classifiers based on age alone vs age plus differentially-expressed proteins. The proteins add significantly to the logistic regression model compared with age alone (p<0.001). Blue=age alone; red=age plus first two principal components.

FIG. 4. Functional interaction network of differentially abundant proteins visualized using STRING. Each node represents a protein, and each edge an interaction. The interactions are coded by color and effects (positive, negative, unspecified) as shown in the legend. The minimum required interaction score was set to high confidence (0.7). See TABLE 2 for protein symbol reference.

FIG. 5. Exemplary peptides for measuring expression of ACVS-related proteins.

FIG. 6. Participant flow diagram for SpecTRA cohorts. TGA=Transient Global Amnesia.

FIG. 7. Optimism corrected ROC curves of the penalized logistic regression model.

FIG. 8. Six exemplary protein targets for iMALDI.

FIG. 9. Seven additional exemplary iMALDI targets.

FIG. 10. Model performance in the three performance target scenarios.

Confidence intervals (CIs) computed by Bootstrap=stratified bootstrap method and Standard=standard method (\hat{p}±1.96*se(\hat{p})).

FIG. 11. Negative (NPV) and positive (PPV) predictive values for the models in each of the three performance target scenarios.

FIGS. 12A-D. ROC plots of each of four exemplary algorithms. FIG. 12 shows the ROC plots for a 15 protein panel GLM (FIG. 12A), 16 protein panel GLM (FIG. 12B), 5 protein panel GLM (FIG. 12C), and 4 protein panel GLM (FIG. 12D). Scenario A: replace M/S score for detection of ACVS; models exclude M/S evaluated in full set of Examples 8 and 9 patients. Scenario B: upgrade stroke unit referral urgency for M/S-negative; models exclude M/S evaluated in the non-M/S subset of Examples 8 and 9 patients. Scenario C: upgrade medical imaging urgency; models include M/S evaluated in the full set of Study 2 patients.

FIG. 13. Performance of models using data from Examples 3 and 4. Opt=optimism correction, conditional on feature selection already having been performed. * Model performance achieved target; † performance confidence interval encompasses target.

FIG. 14. Exemplary use of the methods described herein for patient triage in a clinical setting.

FIGS. 15A-15D. Candidate proteins to be examined and selected on the basis of a literature review of previously investigated protein biomarkers for TIA/mild stroke.

FIGS. 16A-16B. Univariate analysis of peptides for first blood draw using robust logistic regression to predict diagnosis (0=Mimic, 1=ACVS). B=coefficient estimate, OR=odds ratio, CI=confidence interval.

FIGS. 17A-17B. Summary of a validation experiment for the exemplary iMALDI panel in TABLE 13.

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a protein” includes single or plural proteins and is considered equivalent to the phrase “comprising at least one protein.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GENBANK® Accession Nos. referred to herein are the sequences available at least as early as Mar. 17, 2017. All references and GENBANK® Accession numbers cited herein, and the sequences associated therewith, are incorporated by reference in their entireties.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided.

Acute cerebrovascular syndrome (ACVS): A clinical concept that includes patients presenting symptoms within the first 24 hours from onset and prior to the completion of imaging studies with potential diagnoses of cerebral infarction (including acute ischemic stroke, AIS), transient ischemic attack (TIA), and hemorrhage.

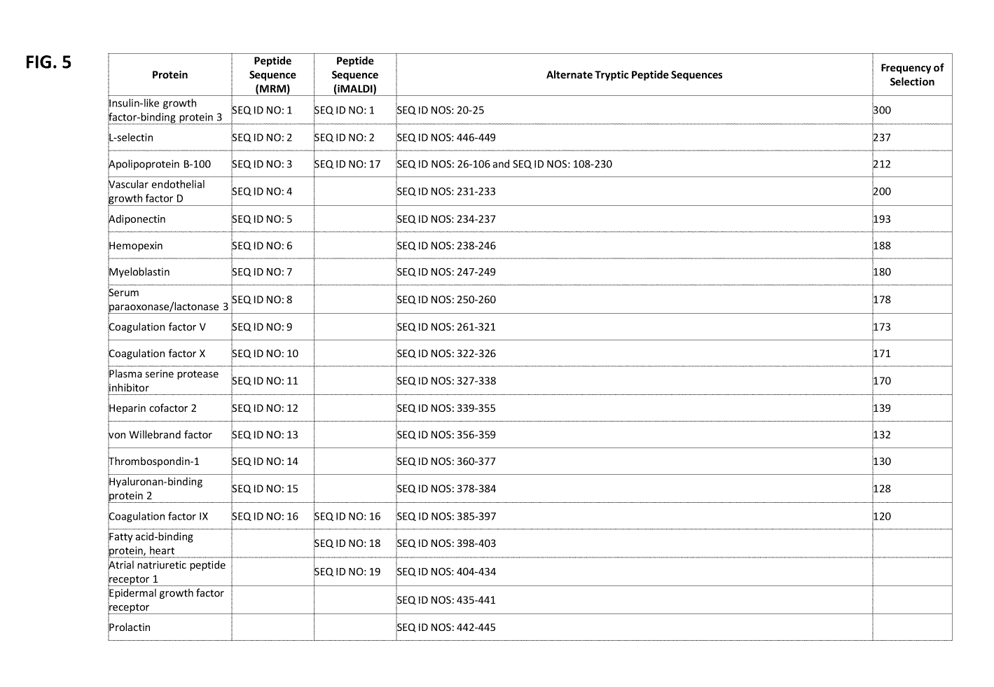

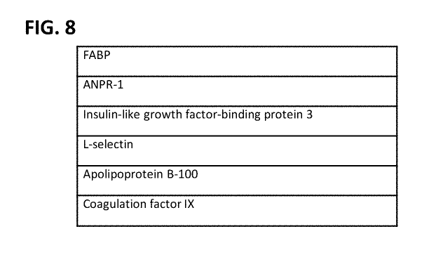

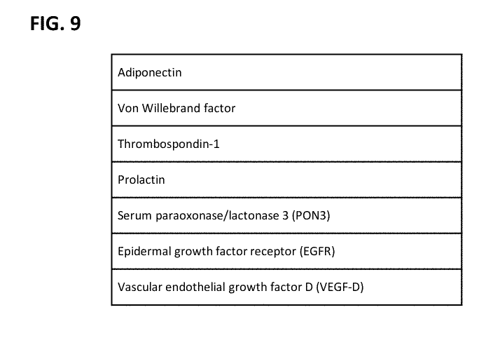

ACVS-biomarker, protein or peptide: A molecule whose expression is affected by an ACVS event. Such molecules include, for instance, nucleic acid sequences (such as DNA, cDNA, or mRNAs), peptides, and proteins. Specific examples include those listed in FIG. 5, FIG. 8, and FIG. 9 as well as TABLE 5.

Adiponectin (ADPN): Also known as adipocyte-, clq-, and collagen domain-containing (ADIPOQ); adipose most abundant gene transcript 1 (APM1), gelatin-binding protein, 28-KD (GBP28); ACRP30; adipocyte-specific secretory protein (ACDC; e.g., OMIM 605441), ADPN is a hormone secreted by adipocytes that regulates energy homeostasis and glucose and lipid metabolism. ADPN exhibits anti-inflammatory effects on the vascular wall and regulates glucose metabolism and insulin sensitivity, and ADPN plays a role in obesity and type II diabetes. Further, ADPN may protect the heart from ischemia-reperfusion injury and modulate oxidant stress.

Includes ADPN nucleic acid molecules and proteins. ADPN sequences are publicly available. For example, GENBANK® Accession Nos. NM_004797.3, NM_144744.3, and NM_009605.5 disclose exemplary human, rat, and mouse ADPN nucleotide sequences, respectively, and GENBANK® Accession Nos. NP_001171271.1, NP_653345.1, and NP_033735.3 disclose exemplary human, rat, and mouse ADPN protein sequences, respectively. One of ordinary skill in the art can identify additional ADPN nucleic acid and protein sequences, including ADPN variants that retain ADPN biological activity (such as having differentially expressed peptides in a subject with ACVS).

Administration: To provide or give a subject a therapeutic intervention, such as a therapeutic drug, procedure, or protocol. Exemplary routes of administration for drug therapy include, but are not limited to, oral, injection (such as subcutaneous, intramuscular, intradermal, intraperitoneal, intratumoral, and intravenous), sublingual, rectal, transdermal, intranasal, and inhalation routes.

Anti-coagulants: Agents that decrease or prevent blood clotting. Anticoagulants can avoid the formation of new clots, and prevent existing clots from growing (extending), for example by decreasing or stopping the production of proteins necessary for blood to clot. Examples include, but are not limited to, aspirin, heparin, ximelagatran, and warfarin (Coumadin). Administration of anticoagulants is one treatment for ischemic stroke, for example to prevent further strokes. A particular type of anti-coagulant are anti-platelet agents, which can also be used to prevent further strokes from occurring and include aspirin, clopidogrel (Plavix), aspirin/dipyridamole combination (Aggrenox), and ticlopidine (Ticlid). Other agents used to prevent stroke recurrence are antihypertensive drugs and lipid-lowering agents such as statins.

Apolipoprotein B (apoB100): Also known as APOB, ag lipoprotein, low density lipoprotein cholesterol level quantitative trait locus 4 (LDLCQ4; e.g., OMIM 107730), apoB100 is synthesized by the liver. Further, polymorphisms and mutations are implicated in gallbladder cancer, hypercholesterolemia, and hypobetalipoproteinemia.

Includes apoB100 nucleic acid molecules and proteins. ApoB100 sequences are publicly available. For example, GENBANK® Accession Nos. AH003569.2, NM_019287.2, and NM_009693.2 disclose exemplary human, rat, and mouse apoB100 nucleotide sequences, respectively, and GENBANK® Accession Nos. NP_000375.2, NP_062160.2, and NP_033823.2 disclose exemplary human, rat, and mouse apoB100 protein sequences, respectively. One of ordinary skill in the art can identify additional apoB100 nucleic acid and protein sequences, including apoB100 variants that retain apoB100 biological activity (such as having differentially expressed peptides in a subject with ACVS).

Atrial natriuretic peptide receptor-1 (ANPR-1): Also known as atrial natriuretic peptide receptor, type A (ANPRA, NPRA); atrionatriuretic peptide receptor, type A; natriuretic peptide receptor A/guanylate cyclase A (NPR1); and guanylyl cyclase 2A (GUC2A; e.g., OMIM 108960), ANPR-1 is a membrane-bound guanylate cyclase that serves as the receptor for both atrial and brain natriuretic peptides. ANPR-1 typically binds natriuretic peptides in the kidney, vascular tissue, and adrenal gland, which induces a blood pressure-lowering effect; ANPR-1 is also found in lungs and adipocytes.

Includes ANPR-1 nucleic acid molecules and proteins. ANPR-1 sequences are publicly available. For example, GENBANK® Accession Nos. NM_000906.3, NM_012613.1, and NM_008727.5 disclose exemplary human, rat, and mouse ANPR-1 nucleotide sequences, respectively, and GENBANK® Accession Nos. NP_000897.3, NP_036745.1, and NP_032753.5 disclose exemplary human, rat, and mouse ANPR-1 protein sequences, respectively. One of ordinary skill in the art can identify additional ANPR-1 nucleic acid and protein sequences, including ANPR-1 variants that retain ANPR-1 biological activity (such as having differentially expressed peptides in a subject with ACVS).

Clinical indications of stroke: One or more signs or symptoms that are associated with a subject having (or had) a stroke, such as an ischemic stroke. Particular examples include, but are not limited to: headache, sensory loss (such as numbness, particularly confined to one side of the body or face), paralysis (such as hemiparesis), pupillary changes, blindness (including bilateral blindness), ataxia, memory impairment, dysarthria, somnolence, and other effects on the central nervous system recognized by those of skill in the art.

Coagulation factor V (F5): Also known as factor V, protein C cofactor (PCCF), activated protein C cofactor (APC cofactor), and labile factor (e.g., OMIM 612309), F5 is a plasma glycoprotein that remains inactive until converted to the active form (factor Va) by thrombin. F5 mutations can lead to hemorrhagic disease or thrombosis.

Includes F5 nucleic acid molecules and proteins. F5 sequences are publicly available. For example, GENBANK® Accession Nos. AH005274.2, NM_001047878.1, and NM_007976.3 disclose exemplary human, rat, and mouse F5 nucleotide sequences, respectively, and GENBANK® Accession Nos. NP_000121.2, NP_001041343.1, and NP_032002.1 disclose exemplary human, rat, and mouse F5 protein sequences, respectively. One of ordinary skill in the art can identify additional F5 nucleic acid and protein sequences, including F5 variants that retain F5 biological activity (such as having differentially expressed peptides in a subject with ACVS).

Coagulation factor IX (F9): Also known as factor IX and plasma thromboplastin component (PTC; e.g., OMIM 300746), F9 remains inactive until proteolytic release of its activation peptide, whereupon, it assume an active serine protease conformation. F9 plays a role in the blood coagulation cascade by activating factor X. Further, F9 inhibitors can function as anticoagulants, and F9 defects and mutations are implicated in thrombophilia and hemophilia.

Includes F9 nucleic acid molecules and proteins. F9 sequences are publicly available. For example, GENBANK® Accession Nos. M35672.1, NM_031540.1, and M23109.1 disclose exemplary human, rat, and mouse F9 nucleotide sequences, respectively, and GENBANK® Accession Nos. AAB28588.1, NP_113728.1, and NP_032005.1 disclose exemplary human, rat, and mouse F9 protein sequences, respectively. One of ordinary skill in the art can identify additional F9 nucleic acid and protein sequences, including F9 variants that retain F9 biological activity (such as having differentially expressed peptides in a subject with ACVS).

Coagulation factor X (F10): F10 (e.g., OMIM 613872) is a serine protease that play a pivotal role in clotting and is activated either by a contact-activated (intrinsic) pathway or by a tissue factor (extrinsic) pathway; the activated form of F10 (factor Xa) then activates prothrombin, forming the effector enzyme of the coagulation cascade. A deficiency in F10 can cause prolonged bleeding.

Includes F10 nucleic acid molecules and proteins. F10 sequences are publicly available. For example, GENBANK® Accession Nos. M57285.1, NM_017143.2, and AJ222677.1 disclose exemplary human, rat, and mouse F10 nucleotide sequences, respectively, and GENBANK® Accession Nos. NP_000495.1, NP_058839.1, and NP_031998.3 disclose exemplary human, rat, and mouse F10 protein sequences, respectively. One of ordinary skill in the art can identify additional F10 nucleic acid and protein sequences, including F10 variants that retain F10 biological activity (such as having differentially expressed peptides in a subject with ACVS).

Differential expression or altered expression: A difference, such as an increase or decrease, in the conversion of the information encoded in a gene (such as a ACVS-related gene) into messenger RNA, the conversion of mRNA to a protein, or both. In some examples, the difference is relative to a control or reference value, such as a cut-off value of expression for each marker. Detecting differential expression can include measuring a change in gene or protein expression, such as a change in expression of one or more ACVS-related genes or proteins disclosed herein.

Control: A reference standard. In some embodiments, the control is a sample obtained from one or more subjects without ACVS (e.g., a blood sample from one or more subjects without ACVS, such as a blood sample from one or more subjects without transient ischemic attack, TIA). In some embodiments, the control includes more than one subject, such as a cohort of control subjects. In still further embodiments, the control is a reference value, range of values, or threshold of values, such as from one or more subjects (e.g., a cohort). The historical control or standard (e.g., a previously tested control sample with a known prognosis or outcome or group of samples that represent baseline or normal values).

Downregulated or deactivation: When used in reference to the expression of a nucleic acid molecule, such as a gene, refers to any process which results in a decrease in the production of a gene product. A gene product can be RNA (such as mRNA, rRNA, tRNA, and structural RNA) peptide, or protein. Therefore, gene downregulation or deactivation includes processes that decrease transcription of a gene or translation of mRNA.

Gene downregulation includes any detectable decrease in the production of a gene product, such as a protein or peptide. In certain examples, production of a gene product decreases by at least 20%, at least 30%, at least 40%, at least 50%, at least 60%, at least 75%, at least 80%, at least 90%, at least 95%, at least 99%, as compared to a control.

Epidermal growth factor receptor (EGFR): Also known as V-ERB-B avian erythroblastic leukemia viral oncogene homolog, oncogene ERBB, ERBB1, HER1 species antigen 7 (SA7; e.g., OMIM 131550), EGFR is a cell signaling molecule involved in diverse cellular functions, including cell proliferation, differentiation, motility, and survival, and in tissue development. EGFR may also play a role in lung, brain, and breast cancer as well as recovery after brain injury.

Includes EGFR nucleic acid molecules and proteins. EGFR sequences are publicly available. For example, GENBANK® Accession Nos. NM_001346941.1, AB025197.1, and AF125256.1 disclose exemplary human, rat, and mouse EGFR nucleotide sequences, respectively, and GENBANK® Accession Nos. AAH94761.1, NP_113695.1, and NP_997538.1 disclose exemplary human, rat, and mouse EGFR protein sequences, respectively. One of ordinary skill in the art can identify additional EGFR nucleic acid and protein sequences, including EGFR variants that retain EGFR biological activity (such as having differentially expressed peptides in a subject with ACVS).

Evaluating ACVS: To determine whether an ACVS event has occurred in a subject (such as a TIA), to determine the severity of an ACVS event, to determine the likely neurological recovery of a subject who has had an ACVS event, or combinations thereof.

Expression: The process by which the coded information of a gene is converted into an operational, non-operational, or structural part of a cell, such as the synthesis of a protein. Gene expression can be influenced by external signals. For instance, exposure of a cell to a hormone may stimulate expression of a hormone-induced gene. Different types of cells can respond differently to an identical signal. Expression of a gene also can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced.

The expression of a nucleic acid molecule can be altered relative to a normal (wild type) nucleic acid molecule. Alterations in gene expression, such as differential expression, includes but is not limited to: (1) overexpression; (2) underexpression; or (3) suppression of expression. Alternations in the expression of a nucleic acid molecule can be associated with, and in fact cause, a change in expression of the corresponding protein.

Protein expression can also be altered in some manner to be different from the expression of the protein in a normal (wild type) situation. This includes but is not necessarily limited to: (1) a mutation in the protein such that one or more of the amino acid residues is different; (2) a short deletion or addition of one or a few (such as no more than 10-20) amino acid residues to the sequence of the protein; (3) a longer deletion or addition of amino acid residues (such as at least 20 residues), such that an entire protein domain or sub-domain is removed or added; (4) expression of an increased amount of the protein compared to a control or standard amount; (5) expression of a decreased amount of the protein compared to a control or standard amount; (6) alteration of the subcellular localization or targeting of the protein; (7) alteration of the temporally regulated expression of the protein (such that the protein is expressed when it normally would not be, or alternatively is not expressed when it normally would be); (8) alteration in stability of a protein through increased longevity in the time that the protein remains localized in a cell; and (9) alteration of the localized (such as organ or tissue specific or subcellular localization) expression of the protein (such that the protein is not expressed where it would normally be expressed or is expressed where it normally would not be expressed), each compared to a control or standard. Controls or standards for comparison to a sample, for the determination of differential expression, include samples believed to be normal (in that they are not altered for the desired characteristic, for example a sample from a subject who has not had an ischemic stroke) as well as laboratory values, even though possibly arbitrarily set, keeping in mind that such values can vary from laboratory to laboratory.

Laboratory standards and values may be set based on a known or determined population value and can be supplied in the format of a graph or table that permits comparison of measured, experimentally determined values.

Fatty Acid-Binding Protein 3 (FABP3): Also known as fatty acid-binding protein, muscle and heart; fatty acid-binding protein, skeletal muscle; and mammary-derived growth inhibitor (MDGI; e.g., OMIM 134651), FABP3 is a transport vehicle for fatty acids throughout the cytoplasm and is found in muscle and the heart. FABP3 is released from cardiac myocytes following an ischemic episode and is a biomarker for myocardial infarction.

Includes FABP3 nucleic acid molecules and proteins. FABP3 sequences are publicly available. For example, GENBANK® Accession Nos. CR456867.1, NM_024162.1, and NM_010174.1 disclose exemplary human, rat, and mouse FABP3 nucleotide sequences, respectively, and GENBANK® Accession Nos. NP_001307925.1, EDL80590.1, and NP_034304.1 disclose exemplary human, rat, and mouse FABP3 protein sequences, respectively. One of ordinary skill in the art can identify additional FABP3 nucleic acid and protein sequences, including FABP3 variants that retain FABP3 biological activity (such as having differentially expressed peptides in a subject with ACVS).

Hyaluronan-binding protein 2 (HABP2): Also known as hyaluronic acid-binding protein 2; hyaluronan-binding protein, plasma (PHBP); hepatocyte growth factor activator-like (HGFAL); and factor VII-activating protease (FSAP; e.g., OMIM 603924), HABP2 is expressed in the kidney, liver, and pancreas. Further, HABP2 plays a role in thyroid cancer, and defects in HABP2 can increase cardiovascular risk.

Includes HABP2 nucleic acid molecules and proteins. HABP2 sequences are publicly available. For example, GENBANK® Accession Nos. KR710720.1, NM_001001505.1, and NM_001329935.1 disclose exemplary human, rat, and mouse HABP2 nucleotide sequences, respectively, and GENBANK® Accession Nos. NP_001171131.1, AAI29081.1, and NP_001316864.1 disclose exemplary human, rat, and mouse HABP2 protein sequences, respectively. One of ordinary skill in the art can identify additional HABP2 nucleic acid and protein sequences, including HABP2 variants that retain HABP2 biological activity (such as having differentially expressed peptides in a subject with ACVS).

Hemopexin (HPX): HPX (e.g., OMIM 142290) is a plasma beta-glycoprotein that binds heme with high affinity and transports it to hepatocytes to salvage iron. Low HPX levels can result in hemolysis.

Includes HPX nucleic acid molecules and proteins. HPX sequences are publicly available. For example, GENBANK® Accession Nos. NM_000613.2, NM_053318.1, and NM_017371.2 disclose exemplary human, rat, and mouse HPX nucleotide sequences, respectively, and GENBANK® Accession Nos. AAH05395.1, NP_445770.1, and NP_059067.2 disclose exemplary human, rat, and mouse HPX protein sequences, respectively. One of ordinary skill in the art can identify additional HPX nucleic acid and protein sequences, including HPX variants that retain HPX biological activity (such as having differentially expressed peptides in a subject with ACVS).

Heparin cofactor II (HCF2): Also known as leuserpin 2 (LS2) and SERPIND1 (e.g., OMIM 142360), HCF2 is a serine protease inhibitor in plasma that rapidly inhibits thrombin and exhibits anti-atherogenic activity. Further, an HCF2 deficiency promotes atherogenesis and neointima formation.

Includes HCF2 nucleic acid molecules and proteins. HCF2 sequences are publicly available. For example, GENBANK® Accession Nos. M12849.1, AF096869.1, and AF097643.1 disclose exemplary human, rat, and mouse HCF2 nucleotide sequences, respectively, and GENBANK® Accession Nos. AAA52642.1, NP_077358.1, and AAA18452.1 disclose exemplary human, rat, and mouse HCF2 protein sequences, respectively. One of ordinary skill in the art can identify additional HCF2 nucleic acid and protein sequences, including HCF2 variants that retain HCF2 biological activity (such as having differentially expressed peptides in a subject with ACVS).

Insulin-like growth factor-binding protein 3 (IGFBP3): Also referred to as IBP3 (e.g., OMIM 146732), IGFBP3 functions as the major carrying protein for IGF1 and IGF2 in circulation, modulates IGF bioactivity, and directly inhibits growth in the extravascular tissue compartment, where it's expression is highly regulated. Further, IGFBP3 protects the vasculature from damage by preventing oxygen-induced vessel loss and promoting vascular regrowth after vascular destruction as well as vascular repair after hyperoxic insult.

Includes IGFBP3 nucleic acid molecules and proteins. FABP3 sequences are publicly available. For example, GENBANK® Accession Nos. NM_001013398.1, NM_012588.2, and NM_008343.2 disclose exemplary human, rat, and mouse IGFBP3 nucleotide sequences, respectively, and GENBANK® Accession Nos. NP_001013416.1, AAI28765.1, and AAH58261.1 disclose exemplary human, rat, and mouse IGFBP3 protein sequences, respectively. One of ordinary skill in the art can identify additional IGFBP3 nucleic acid and protein sequences, including IGFBP3 variants that retain IGFBP3 biological activity (such as having differentially expressed peptides in a subject with ACVS).

Ischemic stroke: Infarction of central nervous system tissue. An ischemic stroke occurs when a blood vessel that supplies blood to the brain is blocked or narrowed (as contrasted with a hemorrhagic stroke which develops when an artery in the brain leaks or ruptures and causes bleeding inside the brain tissue or near the surface of the brain). In acute ischemic stroke (AIS), the blockage can be a blood clot that forms or lodges inside the blood vessel (thrombus) or an object (such as an air bubble or piece of tissue) that moves through the blood from another part of the body (embolus). In some examples, ischemic stroke can be treated with an endovascular thrombectomy.

Isolated: An “isolated” biological component (such as a nucleic acid molecule, protein, peptide or cell) has been substantially separated or purified away from other biological components in the cell of the organism, or the organism itself, in which the component naturally occurs, such as other chromosomal and extra-chromosomal DNA and RNA, proteins and cells. Nucleic acid molecules and proteins that have been “isolated” include ADPN-related molecules (such as DNA or RNA) and proteins purified by standard purification methods. The term also embraces nucleic acid molecules, proteins and peptides prepared by recombinant expression in a host cell as well as chemically synthesized nucleic acid molecules and proteins. For example, an isolated protein, such as a ACVS protein or peptide, is one that is substantially separated from other types of proteins or peptides in a cell.

Label: An agent capable of detection, for example by mass spectrometry, ELISA, spectrophotometry, flow cytometry, or microscopy. For example, a label can be attached to a nucleic acid molecule or protein, thereby permitting detection of the nucleic acid molecule or protein. For example, a protein or peptide can be produced as a heavy, stable isotope, but as a protein or peptide with ¹³C or ¹⁵N incorporated as a heavy, stable isotope. Examples of labels include, but are not limited to, radioactive or heavy, stable isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

L-selectin (SELL): Also referred to as lymphocyte adhesion molecule 1, LYAM1, LAM1, LEU8, CD62 antigen ligand, and CD62L (e.g., OMIM 153240), SELL is a cell surface component and a member of an adhesion protein family. Further, SELL plays a role in lymphocyte homing and neutrophil adhesion to the endothelium at sites of inflammation, and trophoblast SELL mediates interactions with the uterus, which includes an adhesion mechanism that may be critical to establishing human pregnancy.

Includes SELL nucleic acid molecules and proteins. SELL sequences are publicly available. For example, GENBANK® Accession Nos. AJ246000.1, NM_019177.3, and NM_011346.2 disclose exemplary human, rat, and mouse SELL nucleotide sequences, respectively, and GENBANK® Accession Nos. CAB55488.1, NP_062050.3, and NP_035476.1 disclose exemplary human, rat, and mouse SELL protein sequences, respectively. One of ordinary skill in the art can identify additional IGFBP3 nucleic acid and protein sequences, including SELL variants that retain SELL biological activity (such as having differentially expressed peptides in a subject with ACVS).

Myeloblastin (MBT): Also known as proteinase 3 (PRTN3, PR3); Wegener autoantigen (P29); azurophil granule protein 7 (AGP7); and serine proteinase, neutrophil (e.g., OMIM 177020), MBT is a neutrophil with serine proteinase activity and antiproliferative properties that is expressed during bone marrow development. Further, MBT may be involved in mucosal inflammation and inflammatory vascular disease.

Includes MBT nucleic acid molecules and proteins. MBT sequences are publicly available. For example, GENBANK® Accession Nos. M75154.1, NM_001024264.1, and NM_011178.2 disclose exemplary human, rat, and mouse MBT nucleotide sequences, respectively, and GENBANK® Accession Nos. NP_002768.3, NP_001019435.1, and NP_035308.2 disclose exemplary human, rat, and mouse MBT protein sequences, respectively. One of ordinary skill in the art can identify additional MBT nucleic acid and protein sequences, including MBT variants that retain MBT biological activity (such as having differentially expressed peptides in a subject with ACVS).

Paraoxonase 3 (PON3): Also known as serum paraoxonase/lactonase 3 (e.g., OMIM 602720), PON3 is a high-density lipoprotein (HDL)-related glycoproteins and member of the paraoxonase family. PON3 is expressed in the liver and kidney and is exclusively localized to the HDL fraction of human plasma. Further, PON3 includes complex carbohydrates and exhibits antioxidant, arylesterase, and lactonase activity. PON3 may also protect against obesity and atherosclerosis.

Includes PON3 nucleic acid molecules and proteins. PON3 sequences are publicly available. For example, GENBANK® Accession Nos. NM_000940.2, NM_001004086.1, and NM_173006.1 disclose exemplary human, rat, and mouse PON3 nucleotide sequences, respectively, and GENBANK® Accession Nos. NP_000931.1, NP_001004086.1, and NP_766594.1 disclose exemplary human, rat, and mouse PON3 protein sequences, respectively. One of ordinary skill in the art can identify additional PON3 nucleic acid and protein sequences, including PON3 variants that retain PON3 biological activity (such as having differentially expressed peptides in a subject with ACVS).

Plasma serine protease inhibitor (SERPINA5): Also known as serpin peptidase inhibitor, Glade A, member 5; plasminogen activator inhibitor-3 (PAI3); and protein C inhibitor (PCI; e.g., OMIM 601841), SERPINA5 inhibits serine proteases and plasminogen activators. Notably, SERPINA5 inhibits protein C, which is a potent anticoagulant.

Includes SERPINA5 nucleic acid molecules and proteins. SERPINA5 sequences are publicly available. For example, GENBANK® Accession Nos. AH004518.2, NM_022957.3, and NM_172953.3 disclose exemplary human, rat, and mouse SERPINA5 nucleotide sequences, respectively, and GENBANK® Accession Nos. NP_000615.3, NP_075246.3, and NP_766541.2 disclose exemplary human, rat, and mouse SERPINA5 protein sequences, respectively. One of ordinary skill in the art can identify additional SERPINA5 nucleic acid and protein sequences, including SERPINA5 variants that retain SERPINA5 biological activity (such as having differentially expressed peptides in a subject with ACVS).

Prolactin (PRL): PRL (e.g., OMIM 176760) is a mammary gland developmental pathway component that plays an important role in regulating adipose tissue metabolism during lactation. Further, processed PRL produces N-terminal fragments with antiangiogenic activity, and PRL has been shown to mediate neurogenesis during pregnancy.

Includes PRL nucleic acid molecules and proteins. PRL sequences are publicly available. For example, GENBANK® Accession Nos. NM_001163558.2, NM_012629.1, and X04418.1 disclose exemplary human, rat, and mouse PRL nucleotide sequences, respectively, and GENBANK® Accession Nos. EAW55435.1, AAI68729.1, and AAH61141.1 disclose exemplary human, rat, and mouse PRL protein sequences, respectively. One of ordinary skill in the art can identify additional PRL nucleic acid and protein sequences, including PRL variants that retain PRL biological activity (such as having differentially expressed peptides in a subject with ACVS).

Sample: A biological specimen containing genomic DNA, RNA (e.g., mRNA), protein, or combinations thereof, obtained from a subject. Examples include, but are not limited to, peripheral blood, serum, plasma, dried blood spots, urine, saliva, tissue biopsy, fine needle aspirate, surgical specimen, and autopsy material. In one example, a sample is a blood sample from a subject with or at risk for ACVS, such as low-, intermediate-, or high-risk ACVS. In some examples, samples are used directly in the methods provided herein. In some examples, samples are manipulated prior to analysis using the disclosed methods, such as through concentrating, filtering, centrifuging, diluting, desalting, denaturing, reducing, alkylating, proteolyzing, or combinations thereof. In some examples, components of the samples are isolated or purified prior to analysis using the disclosed methods, such as isolating cells, proteins, and/or nucleic acid molecules from the samples.

Solid Support: A solid support can be formed from known materials, such as any water-immiscible material. In some examples, suitable characteristics of the material that can be used to form the solid support surface include being capable of covalently attaching an antibody that can bind to a target agent (such as an ACVS-related molecule) with high specificity or, if non-specific binding occurs, being capable of readily removing such non-specific materials from the surface without removing antibody.

The surface of a solid support may be activated by chemical processes that cause covalent linkage of an agent (e.g., an antibody specific for ACVS-related molecule, such as by binding to protein G or protein A covalently coupled to the surface of a solid support) to the support. However, any other suitable method may be used for immobilizing an agent (e.g., antibody) to a solid support including, without limitation, ionic interactions, hydrophobic interactions, covalent interactions, and the like.

In one example, the solid support is a particle, such as a bead. Such particles can be composed of metal (e.g., gold, silver, and/or platinum), metal compound particles (e.g., zinc oxide, zinc sulfide, copper sulfide, and/or cadmium sulfide), non-metal compound (e.g., silica and/or a polymer), as well as magnetic particles (e.g., iron oxide and/or manganese oxide, such as). In some examples, the bead is a latex or glass bead. Exemplary sizes of sold support particles include 5 nm to 5000 nm in diameter (e.g., about at least 0.5, 1, 2, 3, 4, or 5 μm or about 0.5-1, 1-2, 2-3, 3-4, or 4-5 μm or about 1, 2.8, or 4.5 μm in diameter).

In another example, the solid support is a bulk material, such as a paper, membrane, porous material, water immiscible gel, water immiscible ionic liquid, water immiscible polymer (such as an organic polymer), and the like. For example, the solid support can comprises a membrane, such as a semi-porous membrane that allows some materials to pass while others are trapped. In one example, the membrane comprises nitrocellulose.

In one example, the solid support is composed of an organic polymer. Suitable materials for the solid support include, but are not limited to: polypropylene, polyethylene, polybutylene, polyisobutylene, polybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulfornes, hydroxylated biaxially oriented polypropylene, aminated biaxially oriented polypropylene, thiolated biaxially oriented polypropylene, etyleneacrylic acid, thylene methacrylic acid, and blends of copolymers thereof). In one example, a solid support is composed of glass or glass coated with Indium Tin Oxide (ITO). In one example, a solid support is composed of stainless steel.

In yet other examples, the solid support is a material, such as a coating, containing any one or more of or a mixture of the ingredients provided herein.

A wide variety of solid supports can be employed in accordance with the present disclosure. Except as otherwise physically constrained, a solid support may be used in any suitable shapes, such as films, sheets, strips, or plates, or it may be coated onto or bonded or laminated to appropriate inert carriers, such as paper, glass, plastic films, or fabrics.

In one example, solid support is a plate for use in MALDI mass spectrometry. A MALDI plate may be a commercially available with any number of spots, such as 8-, 48-, 96-, or 384-spot plates (e.g., μFocus MALDI plates by Hudson Surface Technology (New Jersey, USA). MALDI plates may be subjected to various processes, including sample spotting, drying, incubation with one or more MALDI matrices (e.g., 1,5-diaminonapthalene, 3,5-dimethoxy-4-hydroxycinnamic acid, α-cyano-4-hydroxycinnamic acid, 2,5-dihydroxybenzoic acid, 9-aminoacridine, Trihydroxyacetophenone, and/or 3-hydroxypicolinic acid), and washing.

Subject: Living multi-cellular vertebrate organisms, a category that includes mammals, such as human and non-human mammals, such as veterinary subjects (for example cats, dogs, cows, sheep, horses, pigs, and mice). In a particular example, a subject is one who has or is at risk for ACVS. In a particular example, a subject is one who is suspected of having ACVS, such as TIA.

Therapeutically effective amount: An amount of a pharmaceutical preparation that alone, or together with a pharmaceutically acceptable carrier or one or more additional therapeutic agents, induces the desired response. A therapeutic agent, such as one used to treat ACVS, is administered in therapeutically effective amounts.

Therapeutic agents can be administered in a single dose, or in several doses, for example daily, during a course of treatment. However, the effective amount can be dependent on the source applied, the subject being treated, the severity and type of the condition being treated, and the manner of administration. Effective amounts of a therapeutic agent can be determined in many different ways, such as assaying for a sign or a symptom of ACVS, such as a TIA. Effective amounts also can be determined through various in vitro, in vivo or in situ assays. For example, a pharmaceutical preparation can decrease one or more symptoms of a ACVS.

Thrombolytics: Agents that promote lysis of thrombi that occlude a cerebral vessel. Examples include, but are not limited to, tissue plasminogen activator (tPA), urokinase, and pro-urokinase. Administration of antithrombotics is one treatment for ischemic stroke, and is often a first line treatment for ischemic stroke. For example, intravenous t-PA can be administered within 3 hours of ischemic stroke onset. Intra-arterial thrombolytic therapy and mechanical clot-retrieval devices can be used to promote rapid lysis of thrombi.

Thrombospondin I (THBS1): Also known as TSP1 (e.g., OMIM 188060), THBS1 is secreted protein that associates with the extracellular matrix and possesses a variety of biologic functions, including a potent antiangiogenic activity. THBS1 is a secondary mediator of the antiangiogenic effects of certain low-dose metronomic chemotherapy regimens, regulates ischemic damage in the kidney, and plays a role in ischemic renal failure pathophysiology.

Includes THBS1 nucleic acid molecules and proteins. THBS1 sequences are publicly available. For example, GENBANK® Accession Nos. M99425.1, NM_001013062.1, and NM_011581.3 disclose exemplary human, rat, and mouse THBS1 nucleotide sequences, respectively, and GENBANK® Accession Nos. AAK34948.1, AAQ14549.1, and AAA50611.1 disclose exemplary human, rat, and mouse THBS1 protein sequences, respectively. One of ordinary skill in the art can identify additional THBS1 nucleic acid and protein sequences, including THBS1 variants that retain THBS1 biological activity (such as having differentially expressed peptides in a subject with ACVS).

Transient ischemic attack (TIA): a transient episode of neurological dysfunction caused by focal brain, spinal cord, or retinal ischemia without acute infarction. The typical duration of a TIA is 1 or 2 hours, but occasionally, prolonged episodes occur. TIAs are often labeled “mini-strokes,” because they can be relatively benign in terms of immediate consequences, but the term “warning stroke” is more appropriate, because they can indicate the likelihood of a coming stroke. Temporary symptoms may occur. The symptoms are similar to an ischemic stroke, but TIA symptoms usually last less than five minutes with an average of about a minute. When a TIA is over, that particular blockage usually causes no permanent injury to the brain.

Treating a disease: “Treatment” refers to a therapeutic intervention that ameliorates a sign or symptom of a disease or pathological condition, such a sign or symptom of ACVS. Treatment can also induce remission or cure of a condition, or can reduce the pathological condition, such as blockage of a blood vessel in the brain. In particular examples, treatment includes preventing a disease, for example by inhibiting the full development of a disease, such as an acute stroke. In other examples, treatment includes a carotid endarterectomy. Prevention of a disease does not require a total absence of disease.

Upregulated or activation: When used in reference to the expression of a nucleic acid molecule, such as a gene, refers to any process which results in an increase in the production of a gene product. A gene product can be RNA (such as mRNA, rRNA, tRNA, and structural RNA) peptide, or protein. Therefore, gene upregulation or activation includes processes that increase transcription of a gene or translation of mRNA.

Examples of processes that increase transcription include those that facilitate formation of a transcription initiation complex, those that increase transcription initiation rate, those that increase transcription elongation rate, those that increase processivity of transcription, and those that relieve transcriptional repression (for example, by blocking the binding of a transcriptional repressor). Gene upregulation can include inhibition of repression as well as stimulation of expression above an existing level. Examples of processes that increase translation include those that increase translational initiation, those that increase translational elongation and those that increase mRNA stability.

Gene upregulation includes any detectable increase in the production of a gene product, such as a protein. In certain examples, production of a gene product increases by at least 20%, at least 30%, at least 40%, at least 50%, at least 60%, at least 75%, at least 80%, at least 90%, at least 95%, at least 99%, at least 100%, at least 2-fold, at least 3-fold, at least 4-fold, or at least 5-fold, as compared to a control.

Vascular endothelial growth factor D (VEGF-D): Also known as fos-induced growth factor (FIGF; e.g., OMIM 300091), VEGF-D modulates endothelial cell growth and function and is expressed at high levels in the lung, heart, small intestine, and fetal lung and at lower levels in the skeletal muscle, colon, and pancreas. VEGF-D can induce tumor angiogenesis.

Includes VEGF-D nucleic acid molecules and proteins. VEGF-D sequences are publicly available. For example, GENBANK® Accession Nos. D89630.1, AY032728.1, and D89628.1 disclose exemplary human, rat, and mouse VEGF-D nucleotide sequences, respectively, and GENBANK® Accession Nos. BAA24264.1, AAK96008.1, and BAA14002.1 disclose exemplary human, rat, and mouse VEGF-D protein sequences, respectively. One of ordinary skill in the art can identify additional VEGF-D nucleic acid and protein sequences, including VEGF-D variants that retain VEGF-D biological activity (such as having differentially expressed peptides in a subject with ACVS).

Von Willebrand factor (VWF): Also known as factor VIII-von Willebrand factor (F8VWF; e.g., OMIM 613160), VWF is a glycoprotein that plays a central role in the blood coagulation system, serving both as a major mediator of platelet-vessel wall interaction and platelet adhesion, and as a carrier for coagulation factor VIII. Abnormal VWF activity results in a bleeding disorder.

Includes VWF nucleic acid molecules and proteins. VWF sequences are publicly available. For example, GENBANK® Accession Nos. K03028.1, AJ224673.1, and NM_011708.4 disclose exemplary human, rat, and mouse VWF nucleotide sequences, respectively, and GENBANK® Accession Nos. NP_000543.2, NP_446341.1, and NP_035838.3 disclose exemplary human, rat, and mouse VWF protein sequences, respectively. One of ordinary skill in the art can identify additional VWF nucleic acid and protein sequences, including VWF variants that retain VWF biological activity (such as having differentially expressed peptides in a subject with ACVS).

### Overview

The management of ACVS is hampered by the lack of robust, accessible, cheap biomarkers. Clinical decisions that could benefit from a blood test include differentiating TIA from its many mimics, providing guidance for thrombolysis and thrombectomy candidate selection, or help with etiological diagnosis. To date, no clear troponin has emerged for such critical decision support. The answer lies in finding patterns of biomarkers rather than single entities, but the majority of publications report a single protein or a small group thereof, typically 2-5. Provided herein is a method using larger data sets.

Out of 141 high-interest proteins, 23 are differentially expressed between stroke and stroke mimic as determined using LC/MRM mass spectrometry. Out of 8 low-abundance proteins, 4 low-abundance proteins are differentially expressed between stroke and stroke mimic as determined using enriched mass spectrometry. Out of 32 low-abundance proteins, 4 low-abundance proteins not measurable by the two MS techniques are differentially expressed between stroke and stroke mimic as determined using ELISA assays. In conjunction with age, these 31 proteins can distinguish stroke from mimic with an AUC of 0.94 in a small sample of forty patients.

The 31 significant proteins are involved in blood coagulation, inflammation, neurovascular unit injury, cell adhesion, and atrial fibrillation. Certain such proteins are involved in cancer signaling pathways. Twenty-one such proteins exhibit high-confidence molecular interactions with one another, as determined using STRING analysis. The significant proteins and pathways highlighted here are consistent with the known biology of how proteins are up- or down-regulated during ischemic stroke, and the discriminative power is high. Furthermore, these proteins support multi-protein panels and using mass spectrometry to assemble larger datasets.

These results support using plasma proteins as biomarkers for ACVS diagnosis and the role of mass spectrometry. Spectrometry for TIA Rapid Assessment (SpecTRA) uses MS to examine peptide biomarker panels that discriminate mild-ACVS from mimic in emergency department triage.

Clinical reliance on concurrent, multiplexed assays including many proteins is economically feasible using MS, and variants of this technology can be used in urgent care.

Methods of Treating Subjects with Acute Cerebrovascular Syndrome (ACVS)

Provided herein are methods of treating subjects (e.g., human or veterinary subjects) with acute cerebrovascular syndrome (ACVS; e.g., subjects with transient ischemic attack, TIA). In some examples, the methods include measuring ACVS-related molecules, such as peptides (e.g., peptides derived from proteins) in a sample obtained from the subject, for example a subject with ACVS (e.g., a subject with TIA). In specific, non-limiting examples, the ACVS-related molecules (e.g. peptides and/or proteins) include TIA-related molecules. The sample obtained from the subject can include any type of sample, such as a biological sample, tissue sample, and/or biological fluid sample. In specific, non-limiting examples, the sample is a blood sample, such as plasma, whole blood, serum, and/or a dried blood spot.

In some examples, the methods include measuring differential expression of the ACVS-related molecules (e.g., peptides and/or proteins) compared to a control. In some examples, the control represents the expression for each of the ACVS-related molecules expected in a sample from a subject who does not have ACVS (e.g., TIA-related molecules expected in a sample from a subject who does not have TIA).

In some examples, the methods can be used to determine whether or not to provide or administer therapeutic intervention to a subject. Thus, if a subject has ACVS (e.g., subjects with TIA), a therapeutic intervention, such as thrombolytic therapy, antiplatelet therapy, anticoagulant therapy, or surgery can be used. Using the results of the disclosed assays help distinguish subjects that are likely to have ACVS (e.g., subjects with TIA) versus those that are not likely to have ACVS offers clinical benefit because, where the subject has ACVS (e.g., a subject with TIA), the methods disclosed allow the subject to be selected for therapeutic intervention.

The methods herein can include measuring or detecting absolute or relative amounts (e.g., the assay can be qualitative or quantitative) of ACVS-related molecules present in a sample (such as a blood sample) obtained from the subject, for example, using proteins and/or peptides derived from proteins and/or antibodies, nucleic acid probes, and/or nucleic acid primers specific for each ACVS-related molecule. In certain examples, the methods include measuring at least 1, at least 2, at least 3, at least 4, at least 5, at least 6, at least 7, at least 8, at least 9 or at least 10, such as about 1, 2, 3, 4, 5, 6, 7, 8, 9, 10, 11, 12, 13, 14, 15, 16, 17, 18, 19, 20, 25, 30, 40, or 50 or about 1-2, 2-6, 5-10, 6-12, 12-20, or 20-50 or about 2, 4, 5, 6, 12, 15, 16, or 20 ACVS-related molecules (e.g., peptides and/or proteins). In some examples, the ACVS-related molecules (e.g., at least two ACVS-related molecules, such as at least two ACVS-related peptides and/or proteins) include fatty acid binding protein 3 (FABP3), atrial natriuretic peptide receptor-1 (ANPR-1), insulin-like growth factor binding protein 3 (IGFBP-3), coagulation factor IX (F9), L-selectin (SELL), apolipoprotein B100 (apoB100), vascular endothelial growth factor D (VEGF-D), adiponectin (ADPN), von Willebrand factor (vWF), thrombospondin-1 (THBS1), prolactin (PRL), serum paraoxonase 3 (PON3), epidermal growth factor receptor (EGFR), hemopexin (HPX), myeloblastin (MBT), coagulation factor V (F5), coagulation factor X (F10), plasma serine protease inhibitor (SERPIN A5), heparin cofactor 2 (HCII), hyaluronan-binding protein 2 (HABP2), or any combination thereof.

In specific, non-limiting examples, the methods include measuring IGFBP-3, F9, SELL, and apoB100. In specific, non-limiting examples, the methods include measuring IGFBP-3, F9, SELL, apoB100, and vWF. In specific, non-limiting examples, the methods include measuring FABP3, ANPR-1, IGFBP-3, F9, SELL, and apoB100. In some specific examples, methods include measuring FABP3, ANPR-1, IGFBP-3, F9, SELL, and apoB100 and at least 1, 2, 3, 4, 5, 6, 7, 8, 9, 10, 11, 12, 13, 14, or 15 or about 1-2, 2-3, 3-4, 4-5, 5-6, 6-7, 7-8, 8-9, 9-10, 10-11, 11-12, 12-13, 13-14, or 14-15 of ADPN, vWF, THBS1, PON3, EGFR, VEGF-D, PRL, adiponectin, HPX, MBT, F5, F10, SERPIN A5, HCII, and HABP2. In some specific examples, the methods include measuring IGFBP-3, F9, SELL, apoB100, adiponectin, vWF, THBS1, PON3, VEGF-D, HPX, myeloblastin, F5, F10, SERPIN A5, HCII, and HABP2. In some specific examples, the methods include measuring IGFBP-3, F9, SELL, apoB100, adiponectin, vWF, PON3, VEGF-D, HPX, myeloblastin, F5, F10, SERPIN A5, HCII, and HABP2.

In some examples, the methods include selecting a subject. For example, the subject can be at risk of ACVS (e.g., a subject at risk of TIA and/or acute ischemic stroke, AIS) or not at risk of ACVS (e.g., a subject not at risk of TIA and/or AIS). In some examples, the presence of ACVS (e.g., TIA and/or AIS) symptoms can be known or unknown. In some examples, the presence of ACVS (e.g., TIA and/or AIS) symptoms can be present or not. Exemplary symptoms of ACVS include symptoms of TIA, such as high clinical risk scores for TIA (e.g., ABCD score, including clinical features, such as hemiparesis, which can include a loss of motor skills, including ataxia or sided weakness in the leg, arm, and/or face; speech disturbance; and/or pusher syndrome, including postural balance loss), positive diffusion-weighted imaging, intracranial or extracranial arterial stenosis, multiple episodes of TIA (e.g., crescendo TIA), non-valvular and valvular atrial fibrillation, and/or hypercoagulability, and symptoms of ischemic stroke (e.g., acute ischemic stroke), such as sudden numbness or weakness (e.g., of the face, arm or leg, especially on one side of the body), sudden confusion, sudden trouble speaking (e.g., aphasia and/or dysarthria), sudden trouble seeing in one or both eyes (e.g., hemispatial neglect), sudden trouble walking, sudden dizziness, sudden loss of balance or coordination, sudden severe headache with no known cause, pain (e.g., in the face, arm, and/or leg), hiccups, nausea, chest pain or palpitations, and/or shortness of breath. In specific, non-limiting examples, the methods include selecting subjects in which the presence of motor weakness, aphasia, and/or dysarthria is unknown and/or is not considered before performing the method. In specific, non-limiting examples, the methods include selecting subjects in which motor weakness, aphasia, and/or dysarthria are not present in the subject. In specific, non-limiting examples, the methods include selecting subjects in which the presence of motor weakness, aphasia, and/or dysarthria in the subject is known before performing the methods. In specific examples, the methods include considering the presence and/or severity of motor weakness, aphasia, and/or dysarthria in the subject in determining the therapeutic intervention provided to the subject.

The methods disclose herein can include measuring ACVS-related molecules using any type of assay. In some examples, the assays include mass spectrometry assays. In specific, non-limiting examples, the mass spectrometry assays include quadrupole analyzer assays and/or immuno matrix-assisted laser desorption/ionization (iMALDI) assays. In some examples, the assay (e.g., a quadrupole and/or an iMALDI assay) include a sample digestion step (e.g., digestion of biological, tissue, and/or biological fluid samples, such as blood samples, including plasma, whole blood, serum, and/or dried blood spots). In specific, non-limiting examples, the assay (e.g., a quadrupole and/or an iMALDI assay) includes digestion of a plasma sample. In some examples, digestion can be automated or not. In some examples, digestion include reagents that disrupt molecular interactions, such as denaturants (e.g., chaotropes and/or detergents, such as urea, sodium dodecyl sulfate, octyl glucoside, tween, zwittergents, guanidinium chloride, or guanidine hydrochloride), reducing agents (e.g., 1,4-dithiothreitol, DTT, and tris(2-carboxyethyl)phosphine, TCEP), and side-chain-blocking reagents (e.g., iodoacetamide or iodoacetic acid; methyl methanethiosulfonate, MMTS; and N-ethylmaleimide, NEM). In some examples, additional reagents can be added, including buffers (e.g., carbonate-/bicarbonate-, glycine-, acetate-, buffered saline-, cacodylate-, tris-, maleate-, citrate-, phosphate-, ammonium-, hepes-, and/or barbital-based buffers) and/or co-solvents (e.g., acetonitrile, dimethyl sulfoxide, methanol, isopropyl alcohol, formamide, and/or tetrafluoroethylene). In some examples, digestion includes digestion at temperatures at least at about 18° C., 20° C., 25° C., 30° C., 35° C., 37° C., 40° C., 42° C., 50, 75, 95, 98, 100, or 110° C. or about 18-20° C., 20-25° C., 25-30° C., 30-35° C., 35-37° C., 37-40° C., 40-42° C., 42-50, 50-75, 70-95, 95-98, 98-100, or 100-110° C. or about 25° C., 37° C., or 110° C. for any length of time, such as overnight or at least about 30 min, 1 hour, 2 hours, 3 hours, 4 hours, 6 hours, 8 hours, 10 hours, 12 hours, 15 hours, 18 hours, 20 hours, or 24 hours or about 30 min-1 hour, 1-2 hours, 2-3 hours, 3-4 hours, 4-6 hours, 6-8 hours, 8-10 hours, 10-12 hours, 12-15 hours, 15-18 hours, 18-20 hours, or 20-24 hours or about overnight to 24 hours or about 18 hours.

In some examples, digestion includes added enzymes, such as proteases and/or nucleases. Any type of protease can be added. Exemplary proteases include Arg-C (i.e., arginyl peptidase, endoproteinase Arg-C, or tissue kallikrein), Asp-N(i.e., endoproteinase Asp-N or peptidyl-Asp metalloendopeptidase), Asp-N(N-terminal Glu; i.e., endoproteinase Asp-N or peptidyl-Asp metalloendopeptidase), BNPS or NCS/urea (3-bromo-3-methyl-2-(2-nitrophenylthio)-3H-indole, BNPS-skatol, or N-chlorosuccinimide/urea), caspase-1 (i.e., ICE or interleukin-1(3-converting enzyme), caspase-10 (i.e., Flice2 or Mch4), caspase-2 (i.e., Ich-1 or Nedd2), caspase-3 (i.e., apopain, CPP32, or yama), caspase-4 (i.e., ICE(rel)II, Ich-2, or TX), caspase-5 (i.e., ICE(rel)III or TY), caspase-5 (i.e., ICE(rel)III or TY), caspase-6 (i.e., Mch2), caspase-7 (i.e., CMH-1, ICE-LAP3, or Mch-3), caspase-8 (i.e., FLICE, MASH, or Mch5), caspase-9 (i.e., ICE-Lap6 or Mch6), chymotrypsin (includes low-specificity chymotrypsin), clostripain (i.e., clostridiopeptidase B), enterokinase (i.e., enteropeptidase), factor Xa (i.e., coagulation factor Xa), Glu-C (i.e., endoproteinase Glu-C, V8 protease, or glutamyl endopeptidase; includes Glu-C with or without an ammonium bicarbonate buffer or a phosphate buffer), granzyme B (i.e., cytotoxic T-lymphocyte proteinase 2, granzyme-2, granzyme B, lymphocyte protease, SECT, or T-cell serine protease 1-3E), granzyme B (i.e., cytotoxic T-lymphocyte proteinase 2, granzyme-2, granzyme B, lymphocyte protease, SECT, or T-cell serine protease 1-3E), hydroxylamine (i.e., hydroxylammonium), iodosobenzoic acid (i.e., 2-iodosobenzoic acid), Lys-C (i.e., endoproteinase Lys-C or Lysyl endopeptidase), Lys-N(i.e., endoproteinase Lys-N, peptidyl-Lys metalloendopeptidase, or armillaria mellea neutral proteinase), pancreatic elastase (i.e., pancreatopeptidase E or elastase-1, includes cysteine-modified Lys-N), pepsin A (i.e., pepsin, includes low-specificity pepsin A), prolyl endopeptidase (i.e., prolyl oligopeptidase or post-proline cleaving enzyme), proteinase K (i.e., endopeptidase K or peptidase K), TEV protease (i.e., tobacco etch virus protease or nuclear-inclusion-a endopeptidase), thermolysin (i.e., thermophilic-bacterial protease), thrombin (i.e., factor IIa), and/or trypsin (i.e., trypsin-1, includes arginine-blocked, cysteine-modified, and lysine-blocked trypsin).

In examples, digestion includes adding a protease to one or more ACVS-related proteins, for example, to derive ACVS-related peptides. In some examples, one or more ACVS-related proteins includes insulin-like growth factor-binding protein 3 (IGFBP3), L-selectin (SELL), apolipoprotein B-100 (apoB100), vascular endothelial growth factor D (VEGF-D), adiponectin (ADPN), hemopexin (HPX), myeloblastin (MBT), serum paraoxonase/lactonase 3 (PON3), coagulation factor V (F5), coagulation factor X (F10), plasma serine protease inhibitor (SERPINAS), heparin cofactor 2 (HCF2), von Willebrand factor (vWF), thrombospondin-1 (THBS1), hyaluronan-binding protein 2 (HABP2), coagulation factor IX (F9), fatty acid binding protein 3 (FABP3), and/or atrial natriuretic peptide receptor-1 (ANPR-1). In specific, non-limiting examples, the digestion includes adding trypsin to ACVS-related proteins to generate ACVS-related peptides, such as SEQ ID NO: 1 (IGFBP3), SEQ ID NO: 2 (SELL), SEQ ID NO: 3 (apoB100), SEQ ID NO: 4 (VEGF-D), SEQ ID NO: 5 (ADPN), SEQ ID NO: 6 (HPX), SEQ ID NO: 7 (MBT), SEQ ID NO: 8 (PON3), SEQ ID NO: 9 (F5), SEQ ID NO: 10 (F10), SEQ ID NO: 11 (SERPINAS), SEQ ID NO: 12 (HCF2), SEQ ID NO: 13 (vWF), SEQ ID NO: 14 (THBS1), SEQ ID NO: 15 (HABP2), and/or SEQ ID NO: 16 (F9), SEQ ID NO: 17 (apoB100), SEQ ID NO: 18 (FABP3), and/or SEQ ID NO: 19 (ANPR-1). Other ACVS-related peptides can be derived using the methods herein, such as the peptides of FIG. 5.

In some examples, the assay (e.g., a quadrupole and/or iMALDI assay) includes adding acids (e.g., organic acids, such as hydrochloric acid, trifluoroacetic acid, 2-nitro-5-thiocyanobenzoic acid, and formic acid), such as to facilitate or arrest proteolysis. In some examples, the assay includes proteolytic reagents (e.g., cyanogen bromide, CNBr or BrCN, includes CNBr with or without acids, such as hydrochloric acid and/or formic acids, and N-Bromosuccinimide, NBS). Adding acids (e.g., formic acid) and/or proteolytic reagents includes adding about at least 0.5%, 1%, 2%, 5%, 10%, 20%, 50%, or 90% or about 0.5-1%, 1-2%, 2-5%, 5-10%, 10-20%, 20-50%, or 50-90% or about 10% acid (e.g., formic acid, for example, to arrest proteolysis).

In some examples, the assay (e.g., a quadrupole-based and/or an iMALDI-based assay) includes enriching ACVS-related molecules (i.e., an enriched assay, such as an enriched mass spectrometry (MS) assay; e.g., an enriched MRM assay or an enriched MRM-MS assay), for example, by adding ACVS-related molecule-capture reagents and/or apparatus, such as peptide-binding reagents (e.g., ACVS-related peptide-binding antibodies), for example, on a solid support (e.g., beads, such as superparamagnetic beads with, for example, recombinant protein G or protein A covalently coupled to the surface, for example, protein G or protein A DYNABEADS®). In some examples, the assay includes enriching ACVS-related molecules, such as ACVS-related molecules bound to ACVS-related molecule-capture reagents and/or apparatus (e.g., ACVS-related peptides bounds to ACVS-related peptide-binding antibodies, such as antibodies bound to the surface of a solid support). Any type of antibody can be used, such as monoclonal and/or polyclonal antibodies. ACVS-related molecules can be further enriched using various processes, for example, ACVS-related molecules bound to a solid support can be washed to remove unbound impurities.

In some examples, the assay can also include suspending and/or mixing ACVS-related molecules, for example, in a medium compatible with mass spectrometry (e.g., MALDI), such as a MALDI matrix. Exemplary matrices include 1,5-diaminonapthalene, 3,5-dimethoxy-4-hydroxycinnamic acid, α-cyano-4-hydroxycinnamic acid, 2,5-dihydroxybenzoic acid, 9-aminoacridine, trihydroxyacetophenone, and 3-hydroxypicolinic acid. In specific, non-limiting examples, the medium includes α-cyano-4-hydroxycinnamic acid (i.e., CHCA or HCCA). Various reagents can be added to the medium (e.g., MALDI matrix) to facilitate suspension, mixing, and/or mass spectrometry compatibility, efficacy, and/or efficiency (e.g., solvents, such as organic solvents, for example, acetonitrile and/or ethanol, and/or ion pairing reagents, such as trifluoroacetic acid, TFA, heptafluorobutyric acid (HFBA), and/or formic acid). In some examples, the medium can also be used to elute ACVS-related molecules, for example, from a solid support. In specific, non-limiting examples, the medium includes matrix (e.g., HCCA) at least at about 0.1, 0.5, 1, 2, 5, 10, 15, 20, 30, or 40 mg/ml or about 0.1-0.5, 0.5-1, 1-2, 2-5, 5-10, 10-15, 15-20, 20-30, or 30-40 mg/ml or about 3 mg/ml. In specific, non-limiting examples, the medium can other reagents, such as an organic solvent (e.g., acetonitrile) at least at about 10%, 20%, 30%, 40%, 50%, 60%, 70%, 80%, 90%, or 95% or about 10-20%, 20-30%, 30-40%, 40-50%, 50-60%, 60-70%, 70-80%, 80-90%, or 90-95% or about 70% and/or an ion-pairing reagent (e.g., TFA) at least at about 0.05%, 0.1%, 0.15%, 0.2%, 0.25%, 0.3%, 0.4%, or 0.5% or about 0.05-0.1%, 0.1-0.15%, 0.15-0.2%, 0.2-0.25%, 0.25-0.3%, 0.3-0.4%, or 0.4-0.5%. In specific, non-limiting examples, the assay includes adding the ACVS-related molecules (e.g., peptides) in the medium (e.g., matrix, such as HCCA; TFA; and/or ACN) to a solid support (e.g., a MALDI plate). In some examples, the assay includes removing some or all of any impurities (e.g., detergents, cell extract, buffers, and/or salts) from the ACVS-related molecules, such as using washing, ion exchange, solid-phase extraction, and/or droplet dialysis. In specific, non-limiting examples, the ACVS-related molecules with the medium can be dried on a solid support (e.g., MALDI plate) and then washed with a solution, such as a solution that includes water, organic solvents, and/or a buffered solution, such as ammonium citrate or ammonium phosphate, for example, at least at about 1, 2, 5, 10, 20, or 25 mM ammonium citrate or ammonium phosphate or about 1-2, 2-5, 5-10, 10-20, 20-25 mM ammonium citrate or ammonium phosphate or about 5 mM ammonium citrate.

In some examples the assay includes a chromatography step. Any type of chromatography can be used, such as liquid chromatography and/or reversed-phase chromatography, for any purpose (e.g., separation or isolation of molecules, such as ACVS-related molecules, or to exchange a solvent). In specific examples, chromatography (e.g., liquid chromatography) can be used to separate ACVS-related molecules in a sample. In specific, non-limiting examples, reversed-phase liquid chromatography can be used to separate ACVS-related molecules (e.g., ACVS-related peptides) in a sample. The chromatography disclosed herein can be used with any type of eluent. In some example, the eluent can be collected in tubes, spotted on a plat (e.g., a MALDI plate), or injected into another application (e.g., a mass spectrometer, such as injection of an ESI sample into a mass spectrometer).

In some examples, the assay includes analyzing a mass spectrometry sample. Any number of samples can be analyzed at once, including a single-sample analysis or a multiplex analysis (i.e., analyzing multiple samples simultaneously). Any type of mass spectrometry sample can be analyzed, such as a MALDI or electrospray ionization (ESI) sample. Any type of mass spectrometer can be used, such as a time of flight (TOF), a quadrupole mass analyzer (e.g., a triple quadrupole mass analyzer), an ion trap, and/or a tandem mass spectrometer, in any mode (e.g., negative or positive ion mode). In specific, non-limiting examples, the assay includes analyzing a MALDI sample, such as dried sample (e.g., a dried sample with ACVS-related molecules and/or matrix) on a MALDI plate, using a MALDI mass spectrometer. In specific, non-limiting examples, the assay includes analyzing an ESI sample (e.g., injected from a liquid chromatography apparatus) using, for example, a quadrupole analyzer. In specific, non-limiting examples, the assay includes analyzing a mass spectrometry sample (e.g., a MALDI or ESI sample) using tandem mass spectrometry, such as using a multiple reaction monitoring (MRM) application. In specific, non-limiting examples, the assay includes using an MRM application that is enriched (i.e., an enriched MRM assay or an enriched MRM-MS assay) for the ACVS-related molecules (e.g., peptides), such as using peptide-binding reagents (e.g., ACVS-related peptide-binding antibodies), for example on a solid support (e.g., beads, such as superparamagnetic beads with, for example, recombinant protein G or protein A covalently coupled to the surface, for example, protein G or protein A DYNABEADS®).

### Evaluating Nucleic Acid Expression

In some examples, expression of nucleic acids (e.g., RNA, mRNA, cDNA, genomic DNA) of ACVS-related molecules, such as the molecules IGFBP3, SELL, apoB100, VEGF-D, ADPN, HPX, MBT, PON3, F5, F10, SERPINA5, HCF2, vWF, THBS1, HABP2, F9, FABP3, and/or ANPR-1, are analyzed and, in some examples, quantified. Suitable samples can include biological samples, tissue samples, or biological fluid samples, such as a blood sample (e.g., plasma, whole blood, serum, or dried blood spots) obtained from a subject having or a subject at risk for ACVS. An increase in the amount of nucleic acid molecules for the ACVS-related molecules, such as IGFBP3, SELL, apoB100, VEGF-D, ADPN, HPX, MBT, PON3, F5, F10, SERPINA5, HCF2, vWF, THBS1, HABP2, F9, FABP3, and/or ANPR-1, in the sample indicates that the subject has ACVS as described herein. In some examples, expression of the ACVS-related nucleic acid molecule is normalized to expression in the sample (such as by measuring cDNA, genomic DNA, or mRNA in the sample). In some examples, the assay is multiplexed, in that expression of several nucleic acids are detected simultaneously or contemporaneously (Quek et al., Prostate 75:1886-95, 2015, incorporated herein by reference).

Nucleic acid molecules can be isolated from a sample from a subject having or a subject at risk for ACVS, such as a biological sample, tissue sample, or biological fluid sample, including blood samples (e.g., plasma, whole blood, serum, or dried blood spots). In one example, RNA isolation is performed using a purification kit, buffer set, and protease from commercial manufacturers, such as QIAGEN®, according to the manufacturer's instructions. RNA prepared from a biological sample can be isolated, for example, by guanidinium thiocyanate-phenol-chloroform extraction, and oligp(dT)-cellulose chromatography (e.g., Tan et al., J Biomed Biotechnol., 2009: 574398, 10 pages, incorporated herein by reference in its entirety).

Methods of gene expression profiling include methods based on hybridization analysis of polynucleotides, methods based on sequencing of polynucleotides, and other methods in the art. In some examples, mRNA expression is quantified using northern blotting or in situ hybridization; RNAse protection assays, or PCR-based methods, such as reverse transcription polymerase chain reaction (RT-PCR) or real time quantitative RT-PCR. Alternatively, antibodies can be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE) and gene expression analysis by massively parallel signature sequencing (MPSS).

### Evaluating Protein Expression

In some examples, protein expression of ACVS-related molecules, such as IGFBP3, SELL, apoB100, VEGF-D, ADPN, HPX, MBT, PON3, F5, F10, SERPINAS, HCF2, vWF, THBS1, HABP2, F9, FABP3, and/or ANPR-1, is analyzed and, in some examples, quantified. Suitable samples include biological samples, tissue samples, or biological fluid samples, such as a blood sample (e.g., plasma, whole blood, serum, or dried blood spots), obtained from a subject having or a subject at risk for ACVS. An increase in the amount of ACVSr-related proteins, such as IGFBP3, SELL, apoB100, VEGF-D, ADPN, HPX, MBT, PON3, F5, F10, SERPINAS, HCF2, vWF, THBS1, HABP2, F9, FABP3, and/or ANPR-1 proteins, in the sample indicates that the subject has ACVS, as described herein. In some examples, the assay is multiplexed, in that expression of several proteins is detected simultaneously or contemporaneously.

The expression of ACVS-related molecules, such as 2, 3, 4, 5, 6, 7, 8, 9, 10, 11, 12, 13, 14, 15, 16, 17, or 18 of IGFBP3, SELL, apoB100, VEGF-D, ADPN, HPX, MBT, PON3, F5, F10, SERPINAS, HCF2, vWF, THBS1, HABP2, F9, FABP3, and/or ANPR-1, can be measured using any of a number of techniques, such as direct physical measurements (e.g., mass spectrometry) or binding assays (e.g., immunoassays, agglutination assays, and immunochromatographic assays, such as ELISA, Western blot, or RIA assay). Immunohistochemical techniques can also be utilized for protein detection and quantification.

The method can include measuring or detecting a signal that results from a chemical reaction, e.g., a change in optical absorbance, a change in fluorescence, the generation of chemiluminescence or electrochemiluminescence, a change in reflectivity, refractive index or light scattering, the accumulation or release of detectable labels from the surface, the oxidation or reduction or redox species, an electrical current or potential, changes in magnetic fields, etc. Suitable detection techniques can detect binding events by measuring the participation of labeled binding reagents through the measurement of the labels via their photoluminescence (e.g., via measurement of fluorescence, time-resolved fluorescence, evanescent wave fluorescence, up-converting phosphors, multi-photon fluorescence, etc.), chemiluminescence, electrochemiluminescence, light scattering, optical absorbance, radioactivity, magnetic fields, enzymatic activity (e.g., by measuring enzyme activity through enzymatic reactions that cause changes in optical absorbance or fluorescence or cause the emission of chemiluminescence). In some examples, detection techniques are used that do not require the use of labels, e.g., techniques based on measuring mass (e.g., surface acoustic wave measurements), refractive index (e.g., surface plasmon resonance measurements), or the inherent luminescence of an analyte, such as an ACVS-related molecule, for example, IGFBP3, SELL, apoB100, VEGF-D, ADPN, HPX, MBT, PON3, F5, F10, SERPINAS, HCF2, vWF, THBS1, HABP2, F9, FABP3, and/or ANPR-1.

For the purposes of quantitating proteins, a biological sample of the subject that includes cellular proteins (e.g., a blood sample, such as plasma, whole blood, serum, or dried blood spots) can be used. Quantitation of ACVS-related proteins, such as IGFBP3, SELL, apoB100, VEGF-D, ADPN, HPX, MBT, PON3, F5, F10, SERPINAS, HCF2, vWF, THBS1, HABP2, F9, FABP3, and/or ANPR-1 proteins, can be achieved by immunoassay. The amount of ACVS-related proteins, such as IGFBP3, SELL, apoB100, VEGF-D, ADPN, HPX, MBT, PON3, F5, F10, SERPINAS, HCF2, vWF, THBS1, HABP2, F9, FABP3, and/or ANPR-1 proteins, can be assessed in the sample, for example by contacting the sample with appropriate antibodies (or antibody fragments) specific for each protein, and then detecting a signal (for example present directly or indirectly on the antibody, for example by the use of a labeled secondary antibody).

In one example, an electrochemiluminescence immunoassay is used, such as the V-PLEX™ system (Meso Scale Diagnostics, Rockville, Md.). In such assays, the primary antibodies for ACVS-related proteins, such as IGFBP3, SELL, apoB100, VEGF-D, ADPN, HPX, MBT, PON3, F5, F10, SERPINA5, HCF2, vWF, THBS1, HABP2, F9, FABP3, and/or ANPR-1 proteins, (or the corresponding secondary antibodies) are labeled with an electrochemiluminescent label.

Quantitative spectroscopic approaches methods, such as MALDI (e.g., iMALDI), tandem mass spectrometry, and/or quadrupole-based mass spectrometry, can be used to analyze expression of ACVS-related proteins, such as IGFBP3, SELL, apoB100, VEGF-D, ADPN, HPX, MBT, PON3, F5, F10, SERPINAS, HCF2, vWF, THBS1, HABP2, F9, FABP3, and/or ANPR-1 proteins, in, for example, a blood sample obtained from a subject having or a subject at risk for ACVS.

In one example, LC-MRM (liquid chromatography-multiple reaction monitoring) may be used to detect protein expression, for example, by using a triple quadrupole spectrometer (see, e.g., U.S. Pub. No. 2013/0203096). LC-MRM is a liquid chromatography method that can be used for high-throughput selective and sensitive detection of molecules, such as ACVS-related proteins, for example, IGFBP3, SELL, apoB100, VEGF-D, ADPN, HPX, MBT, PON3, F5, F10, SERPINAS, HCF2, vWF, THBS1, HABP2, F9, FABP3, and/or ANPR-1.

In some examples, analysis and/or measurement of ACVS-related molecules includes enriching a sample for ACVS-related molecules. In specific examples, enriching includes adding ACVS-related molecule-capture reagents and/or apparatus, such as peptide-binding reagents (e.g., ACVS-related peptide-binding antibodies), for example, on a solid support (e.g., beads, such as superparamagnetic beads with, for example, recombinant protein G or protein A covalently coupled to the surface, for example, protein G or protein A DYNABEADS®). In specific examples, enriched samples can be measured using any of number of techniques, such as an MRM assay (i.e., an enriched MRM assay or an enriched MRM-MS assay; e.g., an enriched LC-MRM assay).

Therefore, in a particular example, the analytes include ACVS-related marker proteins and/or peptides thereof, such as IGFBP3, SELL, apoB100, VEGF-D, ADPN, HPX, MBT, PON3, F5, F10, SERPINA5, HCF2, vWF, THBS1, HABP2, F9, FABP3, and/or ANPR-1 proteins and/or peptides thereof. In other examples, the fractionated and pooled analytes consist essentially of or consist of IGFBP3, SELL, apoB100, VEGF-D, ADPN, HPX, MBT, PON3, F5, F10, SERPINA5, HCF2, vWF, THBS1, HABP2, F9, FABP3, and/or ANPR-1 proteins or peptides thereof or of the combinations of proteins or peptides listed in FIG. 5. In this context, “consists essentially of” indicates that the fractionated and pooled analytes do not include other ACVS-related proteins that can be used to accurately predict ACVS, but can include other ACVS molecules, such as protein expression controls.

Therefore, in a particular example, the target analytes include ACVS-related proteins and/or surrogate thereof, such as IGFBP3, SELL, apoB100, VEGF-D, ADPN, HPX, MBT, PON3, F5, F10, SERPINAS, HCF2, vWF, THBS1, HABP2, F9, FABP3, and/or ANPR-1 proteins and/or peptides thereof. In other examples, the target analytes consist essentially of or consist of IGFBP3, SELL, apoB100, VEGF-D, ADPN, HPX, MBT, PON3, F5, F10, SERPINAS, HCF2, vWF, THBS1, HABP2, F9, FABP3, and/or ANPR-1 proteins or peptides thereof; of the combinations of proteins or surrogate peptides listed in FIG. 5. In this context “consists essentially of” indicates that the target analytes do not include other ACVS-related marker proteins that can be used to accurately predict ACVS, but can include other ACVS molecules, such as ACVS protein expression controls.

In a further example, surface-enhanced laser desorption-ionization time-of-flight (SELDI-TOF) mass spectrometry is used to detect protein expression, for example by using the ProteinChip™ (Ciphergen Biosystems, Palo Alto, Calif.).

### ACVS-Related Molecules

The disclosed ACVS-related molecules include IGFBP3, SELL, apoB100, VEGF-D, ADPN, HPX, MBT, PON3, F5, F10, SERPINA5, HCF2, vWF, THBS1, HABP2, F9, FABP3, and/or ANPR-1. One or more of the disclosed ACVS-related molecules can be used alone or in any combination. The molecules can include proteins, peptides (e.g., peptides listed in FIG. 5), and nucleic acids.

In some embodiments, the ACVS-related molecules include one or more TIA-related molecules. Exemplary TIA-related molecules include IGFBP3, SELL, apoB100, VEGF-D, ADPN, HPX, MBT, PON3, F5, F10, SERPINA5, HCF2, vWF, THBS1, HABP2, and F9.

In some embodiments, one of the disclosed ACVS-related molecules includes IGFBP3 (e.g., SEQ ID NO: 1). In some embodiments, one of the disclosed ACVS-related molecules includes SELL (e.g., SEQ ID NO: 2). In some embodiments, one of the disclosed ACVS-related molecules includes apoB100 (e.g., SEQ ID NOS: 3 and 17). In some embodiments, one of the disclosed ACVS-related molecules includes VEGF-D (e.g., SEQ ID NO: 4). In some embodiments, one of the disclosed ACVS-related molecules includes ADPN (e.g., SEQ ID NO: 5). In some embodiments, one of the disclosed ACVS-related molecules includes HPX (e.g., SEQ ID NO: 6). In some embodiments, one of the disclosed ACVS-related molecules includes MBT (e.g., SEQ ID NO: 7). In some embodiments, one of the disclosed ACVS-related molecules includes PON3 (e.g., SEQ ID NO: 8). In some embodiments, one of the disclosed ACVS-related molecules includes F5 (e.g., SEQ ID NO: 9). In some embodiments, one of the disclosed ACVS-related molecules includes F10 (e.g., SEQ ID NO: 10). In some embodiments, one of the disclosed ACVS-related molecules includes SERPINA5 (e.g., SEQ ID NO: 11). In some embodiments, one of the disclosed ACVS-related molecules includes HCF2 (e.g., SEQ ID NO: 12). In some embodiments, one of the disclosed ACVS-related molecules includes vWF (e.g., SEQ ID NO: 13). In some embodiments, one of the disclosed ACVS-related molecules includes THBS1 (e.g., SEQ ID NO: 14). In some embodiments, one of the disclosed ACVS-related molecules includes HABP2 (e.g., SEQ ID NO: 15). In some embodiments, one of the disclosed ACVS-related molecules includes F9 (e.g., SEQ ID NO: 16). In some embodiments, one of the disclosed ACVS-related molecules includes FABP3 (e.g., SEQ ID NO: 18). In some embodiments, one of the disclosed ACVS-related molecules includes ANPR-1 (e.g., SEQ ID NO: 19). In some examples, combinations of these ACVS-related molecules are used, such as 2, 3, 4, 5, 6, 7, 8, 9, 10, 11, 12, 13, 14, 15, 16, 17, 18, or 19 of these.

Molecules that are similar to the ACVS-related molecules disclosed above can be used as well as fragments thereof that retain biological activity. These molecules may contain variations, substitutions, deletions, or additions. The differences can be in regions not significantly conserved among different species. Such regions can be identified by aligning the amino acid sequences of related proteins from various animal species. Generally, the biological effects of a molecule are retained. For example, a molecule at least 75%, 80%, 85%, 90%, 95%, 96%, 97%, 98% or 99% identical to one of these molecules can be utilized. Molecules are of use that include at most 1, 2, 3, 4, 5, 6, 7, 8, 9, or 10 conservative amino acid substitutions. Generally, molecules are of use provided they retain at least 75%, 80%, 85%, 90%, 95%, 96%, 97%, 98% or 99% of the biological function of the native molecule, or have increased biological function as compared to the native molecule.

### Administration of Therapy

Subjects analyzed with the disclosed methods and who are found to have ACVS (e.g., TIA or AIS) can be selected for treatment. For example, subjects with AIS found to have differential expression of IGFBP3, SELL, apoB100, VEGF-D, ADPN, HPX, MBT, PON3, F5, F10, SERPINAS, HCF2, vWF, THBS1, HABP2, F9, FABP3, and/or ANPR-1 can be administered therapy for ACVS (e.g., TIA or AIS). In some examples, subjects with ACVS may be treated using thrombolytic therapy, antiplatelet therapy, anticoagulant therapy, and/or surgery.

In specific examples, thrombolytic therapy can be a treatment for ACVS (e.g., TIA or AIS). Any thrombolytic therapy can be administered (i.e., lytics or “clot busters” to dissolve blood clots that have acutely (suddenly) blocked major arteries or veins). Thrombolytics can be administered either through a peripheral intravenous line or through a catheter. Exemplary agents that can be administered include EMINASE®, anistreplase, RETAVASE®, reteplase, STREPTASE®, streptokinase, kabikinase, t-PA (tissue plasmon activator), ACTIVASE®, TNKASE®, tenecteplase, ABBOKINASE®, KINLYTIC®, rokinase, and urokinase.

In specific examples, antiplatelet therapy can be a treatment for ACVS (e.g., TIA or AIS). Any antiplatelet therapy can be administered (i.e., antiaggregants that decrease platelet aggregation and inhibit thrombus formation). Exemplary agents that can be administered include irreversible cyclooxygenase inhibitors (e.g., aspirin, triflusal, DISGREN®, GRENDIS®, AFLEN®, and TRIFLUX®), adenosine diphosphate (ADP) receptor inhibitors (e.g., clopidogrel, PLAVIX®, prasugrel, EFFIENT®, ticagrelor, BRILINTA®, BRILIQUE®, POSSIA®, ticlopidine, and TICLID®), phosphodiesterase inhibitors (e.g., cilostazol and PLETAL®), protease-activated receptor-1 (PAR-1) antagonists (e.g., vorapaxar, ZONTIVITY®, and SCH 53034), glycoprotein IIB/IIIA inhibitors (e.g., intravenously, such as using abciximab, REOPRO®, c7E3 Fab, eptifibatide, INTEGRILIN®, tirofiban, and AGGRASTAT®), adenosine reuptake inhibitors (e.g., dipyridamole and PERSANTINE®), and thromboxane inhibitors (e.g., thromboxane synthase inhibitors, thromboxane receptor antagonists, seratrodast, AA-2414, bronica, picotamide, and terutroban).

In specific examples, anticoagulant therapy can be a treatment for ACVS (e.g., TIA or AIS). Any anticoagulant therapy can be administered (i.e., blood thinner, or agents that block the activity of coagulation factors, such as specific targets in the coagulation cascade). Exemplary agents that can be administered include coumarins and indandiones (i.e., vitamin K antagonists; e.g., warfarin, JANTOVEN®, and COUMADIN®), factor 10 inhibitors (i.e., inhibitors of coagulation factor 10, F10; e.g., ARIXTRA®, fondaparinux, XARELTO®, rivaroxaban, ELIQUIS®, apixaban, SAVAYSA®, edoxaban, BEVYXXA®, and betrixaban), heparins (e.g., FRAGMIN®, dalteparin, INNOHEP®, tinzaparin, LOVENOX®, enoxaparin, heparin sodium, ORGARAN®, danaparoid, and POSIFLUSH®), and thrombin inhibitors (e.g., ANGIOMAX®, bivalirudin, PRADAXA®, dabigatran, ACOVA®, argatroban, IPRIVASK®, desirudin, REFLUDAN®, and lepirudin).

In specific examples, surgery can be a treatment for ACVS (e.g., TIA or AIS). Exemplary surgical procedures include carotid endarterectomy (e.g., for ACVS with TIA and a blockage in the carotid arteries), carotid artery stenting (i.e., carotid angioplasty), mechanical embolectomy, and cerebral revascularization (i.e., bypass surgery).

The therapy can be administered in cycles (such as 1 to 6 cycles), with a period of treatment (usually 1 to 3 days) followed by a rest period. But some therapies can be administered every day.

### EXAMPLES

### Example 1

### Methods

This example describes the methods used to generate the results described in Example 2.

Differences in the abundance of 141 protein markers were compared to distinguish acute cerebrovascular syndrome (ACVS) from mimic patients. Proteins from the stroke literature and previously studied cardiovascular markers were targeted. All proteins were quantified using mass spectrometry (MS), eight were repeated using antibody protein enrichment with MS, and 32 were repeated using ELISA. Twenty ACVS (NIHSS>5 and <24 hours from onset) and 20 mimics were recruited.

Study Population

Twenty stroke patients were enrolled that presented to the emergency department less than 24 hours after onset and with a National Institute of Health Stroke Scale Score (NIHSS) of >5. Twenty stroke-mimic patients were recruited concurrently from referrals to a stroke rapid assessment unit. Mimic patients were seen within 48-72 hours of reported symptom onset and with a confirmed non-stroke diagnosis by a stroke neurologist. Patients with an uncertain diagnosis or hemorrhagic stroke and those unable to undergo medical imaging were excluded. Patients were enrolled over a two-month period during daytime hours at one hospital.

Study Procedures

Stroke nurses drew blood into 6-mL EDTA tubes using one of three different needle gauges depending on clinical need, including an 18 gauge butterfly with vacutainer (57.5%), a 20 gauge (37.5%), or a 21 gauge (5.0%). The impact of blood drawing techniques (e.g., using a needle gauge) on proteomic levels has been reported ¹⁶. Tubes were immediately iced until centrifuged for 10-15 minutes at 2500-3000 rpm at room temperature. Within 90 minutes of drawing blood, 300 μL of plasma was pipetted into each of 32 (0.50 ml polypropylene) aliquots (3744, Thermo Scientific) per sample. Plasma samples were then stored at −80° C.

Final diagnoses were performed by stroke neurologists, including an etiological classification using the modified TOAST classification system¹⁷.

Proteomic Analyses

The sample preparation protocol for direct LC/MRM-MS analysis is similar to a previously reported procedure¹⁸. Low-abundance endogenous proteins were enriched from plasma using a mixture of 8 antibodies (against EGFR, FABP, IL-6, PECAM, prolactin, protein S100-A12, dickkopf-related protein 1, and glutathione S-transferase P) coupled to protein G-coated magnetic beads (Dynabeads®, ThermoFisher Scientific) before proteolytic digestion with trypsin.

Two mass spectrometry (MS) techniques and ELISA were used to measure the plasma levels of 141 previously reported stroke and cardiovascular markers¹⁹. ELISA was used for 32 proteins anticipated as low-abundance proteins or where no suitable peptides were available for MRM-MS.

Statistical Methods

Descriptive statistics were computed for the clinical variables and protein measurements. After adjusting for age, the average log 2 transformed abundance and relative abundance levels were compared between the groups using a robust regression model to reduce the impact of outliers in the measurements²⁰. A principal components analysis (PCA) was used for dimension reduction and to summarize the information across all proteins²¹. The first two principal components (PCs) were used to visualize distribution of the proteins between mimic and stroke. The improvement made by including these two PCs in a logistic regression model based on age alone was evaluated using a likelihood ratio test. ROC (receiver operating characteristic) analyses were performed using predictions from the logistic regression models, and AUCs (area under the curve) were adjusted for optimism using leave-one-out cross-validation. For this exploratory pilot study, p-values less than 0.1 for ELISA and 0.05 for MRM proteins were considered statistically significant without adjusting for multiple inference. Statistical analyses were performed in R 3.2.2²² using packages, glmnet, epicalc, pROC, pcaMethods, and robustbase. A protein interaction network analysis of the differentially abundant proteins was performed using STRING (version 10.0, string-db.org)²³, which integrates interaction data from several sources with information on physical and functional properties as well as with known and predicted protein interactions. This analysis provides a better understanding of the biological pathways in which the most significant biomarkers are involved.

Plasma Sample Preparation

The sample preparation protocol for LC/MRM-MS analysis is similar to a previously reported procedure [18]. Human plasma proteolytic digests were prepared in a 96-well plate format using a Tecan Freedom Evo robotic liquid handling system (Tecan Group Ltd, Switzerland). Briefly, 10 μL of plasma was denatured and reduced with 9 M urea and 20 mM DL-dithiothreitol for 30 min at 37° C. Alkylation was performed with a 30 min incubation of 50 mM iodoacetamide at room temperature in the dark. The urea concentration was reduced to 0.55 M with 100 mM Tris, pH 8.0 before adding modified porcine trypsin (Worthington Biochemical Corp, NJ, USA) at a 20:1 (sample protein to trypsin) ratio and incubating for 18 hrs at 37° C. Digestion was stopped by adding 1% formic acid. A stable isotope labeled standard peptide mixture, balanced to a standard human plasma sample was added prior to desalting by solid phase extraction (Oasis HLB μ-elution plates, Waters, Milford, Mass., USA). Peptides not detected in the standard human plasma sample were spiked in at 50×LLOQ. The samples were lyophilized to dryness and stored at −80° C. until analyzed by LC/MRM-MS.

Low-abundance endogenous proteins were enriched from plasma using a mixture of 8 antibodies (against EGFR, FABP, IL-6, PECAM, Prolactin, Protein S100-A12, Dickkopf-related protein 1, and Glutathione S-transferase P) coupled to Protein G coated magnetic beads (Dynabeads®, ThermoFisher Scientific) before proteolytic digestion with trypsin. The beads were prepared as follows: beads were first washed to remove detergent that would interfere with downstream MS analysis before coupling to polyclonal IgG capture antibodies through their Fc region. The Protein G beads were then saturated with an equimolar mixture of 8 polyclonal antibodies (pAbs). After washing to remove any unbound pAbs, the bead-Ab complexes were incubated with plasma for 1 hr at 4° C. with rotation. Unbound and weakly bound proteins were removed by washing before elution with 0.1% formic acid. The total wash time was <5 min to reduce losses due to antibody off-rates. The proteins were then digested as described above.

Proteomic Analyses

(I) LC/MRM-MS Analysis

LC/MRM-MS analysis of the plasma digests was performed on an Agilent 1290 Infinity UHPLC system interfaced with an Agilent 6490 triple quadrupole mass spectrometer operating in the positive ion mode. Peptides were separated at 0.4 mL/min using a Zorbax Eclipse Plus C18 RRHD column (150×2.1 mm, 1.8 μm particles; Agilent Technologies, CA, USA) maintained at 50° C. over the 43 min multi-step gradient. Three transitions were monitored at unit resolution for each peptide using optimized collision energy voltages and a minimum dwell time of 12 ms. In total, 870 transitions (representing 141 proteins) were targeted per LC/MRM-MS analysis and a total of 48 transitions (representing 8 proteins) were targeted per enriched LC/MRM-MS analysis.

(II) ELISA

Each of the 32 ELISA assays were run in a 96-well plate format according to each of the manufacturer's instructions. Both the human plasma samples and standard curves were run in duplicate and the resulting measurements were averaged. A simple plate layout randomization scheme with 80 aliquots assigned per plate was used to minimize any bias resulting from aliquot location or processing order. Values below the assay's lower limit of quantitation were replaced with 50% of the smallest observed value for that protein; values above the upper limit of quantitation were replaced with 1.5 times the largest observed value. The log 2 transformed abundance values were used as the response.

Proteomic Data Preparation

MRM data was processed with Skyline Daily3.5.1.9426 analysis software 24 [19]. All peaks were inspected manually to ensure correct chromatographic peak selection and proper peak integration. Peak areas for the endogenous (END) and SIS peptides were measured. Any proteins with over 75% of the peak-area values missing were removed from the analysis, and any relative abundance values reported as zero were replaced with 50% of the smallest observed value for that protein. The log 2 transformed relative abundance values (i.e., log 2 of the END/SIS ratios) were used as the response.

### Example 2

### Results

Thirty-one_proteins (23 by MS, 4 by enriched MS, and 4 by ELISA that were not covered by the MS techniques) exhibited differential abundance between mimic and stroke (each ELISA p<0.1, MS p<0.05). A logistic regression model using the first two principal components of the proteins significantly improved discrimination compared with a model based on age alone (p<0.01, AUC 0.94 vs. 0.78). Significant proteins included markers of inflammation (49%), coagulation (36%), neurovascular unit injury (6%), atrial fibrillation (6%), and other (3%).

TABLE 1 displays the demographic characteristics of the 20 stroke and 20 mimic patients. The stroke patients were older with a slightly higher proportion of males. The three most frequent mimic sub-types were migraine aura without headache, neuropathy, and transient global amnesia. The mean time from symptom onset to blood draw for the stroke events was 10 hours (standard deviation, s.d., 8 hrs). The mean time from blood draw to freeze was 35 minutes (s.d., 12 min) for stroke events and 29 minutes (s.d., 5 min) for mimic events.

TABLE 1
Demographic Summary for the Stroke and Mimic Patients

| | Stroke Patients | Mimic Patients |
|---|---|---|
| | (n = 20) | (n = 20) |
| Male | 8 (40%) | 7 (35%) |
| Age in years, Median [Range] | 77, [49, 95] | 63, [36, 77] |
| Mimic Subtype | | |
| Migraine aura without headache | — | 5 (25%) |
| Neuropathy | — | 4 (20%) |
| Transient global amnesia | — | 4 (20%) |
| Vestibulopathy | — | 2 (10%) |
| Syncope | — | 2 (10%) |
| Multiple sclerosis | — | 1 (5%) |
| Other | — | 2 (10%) |

Each protein was represented by a single peptide for MS measurement, except MMP9 and thrombospondin-1, which were each measured using three peptides. Of the 141 protein targets, four were removed from the statistical analysis prior to computation. MMP9 (represented by the peptide LGLGADVAQVTGALR) and creatine kinase B-type were not detected in any of the samples at the quantification limits of this technique. The protein myosin-11 was only detected in two of the samples, and the protein elastin exhibited the same relative intensities across all samples. Of the 32 proteins quantified using commercial ELISA kits, one protein was excluded from further statistical analyses: the CD40 ligand, which was not detected in any of the 40 samples.

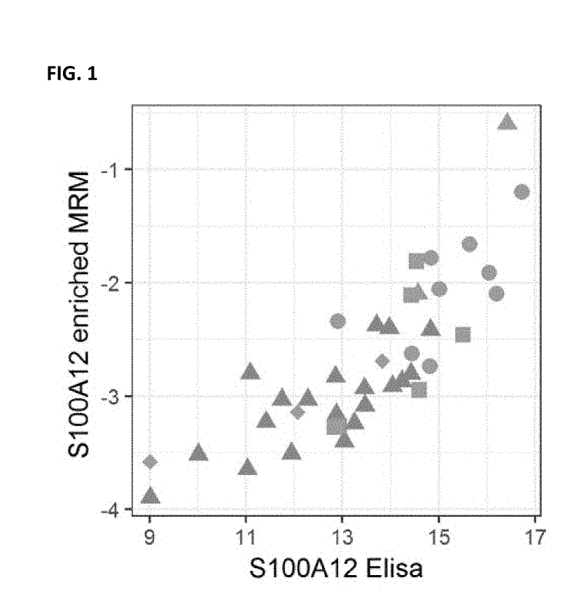

EGFR and S100A12 were measured by both enriched LC-MRM/MS and ELISA. For S100A12, the results for these two techniques were correlated well (Pearson's r=0.819; see FIG. 1); for EGRF, however, the results did not correlate well (r=0.530). The differences may be due to differences between the two capture antibodies (e.g., affinity kinetics and epitope specificity) for the two techniques, but these data were not available from the vendors (see http://stroke.ahajournals.org).

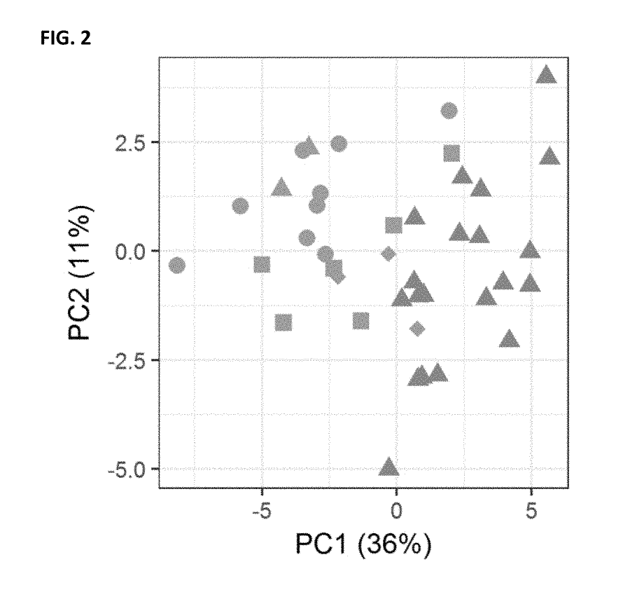

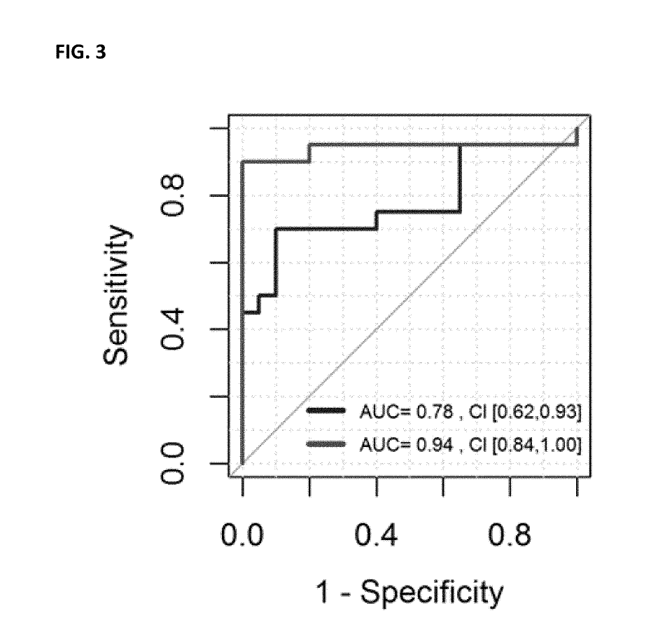

Table 2 lists the 31 proteins with different abundances between the mimic and stroke patients (23 proteins based on LC/MRM-MS, 6 based on ELISA, and 4 based on enriched LC/MRM-MS, two of which were also measured by ELISA), after adjusting for age in robust regression models (ELISA p<0.1, MRM p<0.05). A principal component analysis (PCA) using these 31 differentially expressed proteins generated the first two PCs, which explain 36% (PC1) and 11% (PC2) of the total variability (see FIG. 2). A logistic regression model classifier incorporating PC1 and PC2 in addition to age showed significantly improved performance compared with a model based on age alone (cross-validated AUC 0.94 vs. 0.78; see FIG. 3).

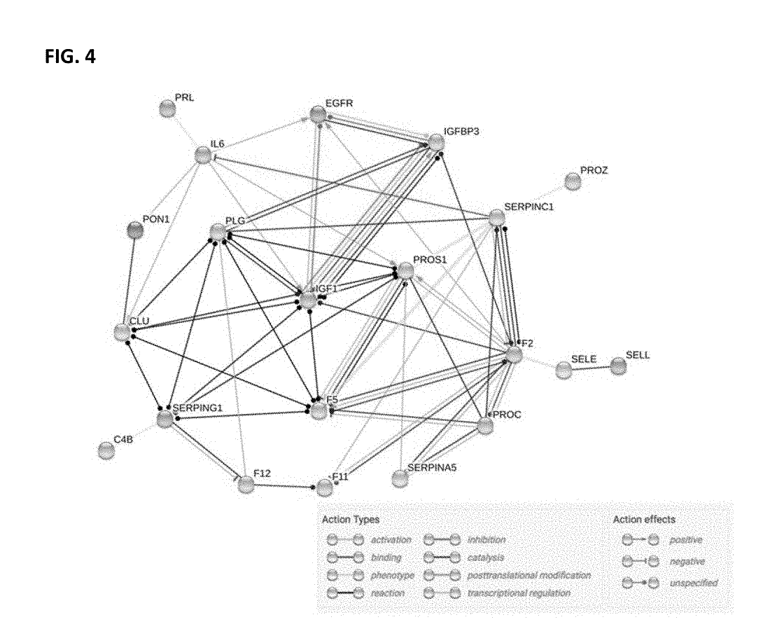

From the list of 31 differentially abundant proteins, the STRING analysis showed that 21 such proteins exhibit high-confidence molecular interactions with other proteins on this list (FIG. 4). The remaining 10 proteins did not interact or exhibited low-confidence interactions.

TABLE 2
Functional summary of the differentially abundant proteins identified by
MRM and ELISA (p-values ELISA p < 0.1, MRM and enriched MRM p < 0.05).

| | | | | Robust reg. |
|---|---|---|---|---|
| | UniProtKB | Marker Type and | Protein | adj for age |
| Protein Name | ID | pathway map if known | Symbol | p-value |
| MRM measured | | | | |
| Apolipoprotein C-I | P02654 | Coag | APOC1 | <0.001 |
| Calponin | P51911 | AF | CNN1 | <0.001 |
| Coagulation factor | P00748 | Coag | F12 | <0.001 |
| XII | | | | |
| E-selectin | P16581 | Infl | SELE | <0.001 |
| C-reactive protein | P02741 | Infl, complement | CRP | 0.001 |
| | | pathways | | |
| Clusterin | P10909 | Infl, complement | CLU | 0.001 |
| | | pathways, canc signal | | |
| IGF-1 | P05019 | Infl, cell adhesion, canc | IGF1 | 0.001 |
| | | signal | | |
| Complement | P0C0L5/ | Infl, complement | C4B | 0.002 |
| component 4b (C4b | P0C0L4 | pathways | | |
| and C4a) | | | | |
| Serum paraoxonase/ | P27169 | Infl | PON1 | 0.002 |
| arylesterase 1 | | | | |
| (Paraoxonase- | | | | |
| PON1) | | | | |
| Prothrombin, | P00734 | Coag, platelet activation, | F2 | 0.004 |
| thrombin | | canc signal | | |
| Plasminogen, | P00747 | Coag, platelet activation, | PLG | 0.005 |
| plasmin, or | | cell adhesion | | |
| angiostatin | | | | |
| Vitamin K- | P07225 | Coag | PROS1 | 0.010 |
| dependent protein S | | | | |
| (Protein S) | | | | |
| Serum paraoxonase/ | Q15166 | Infl | PON3 | 0.013 |
| lactonase 3 | | | | |
| (Paraoxonase- | | | | |
| PON3) | | | | |
| Vitamin K- | P04070 | Coag | PROC | 0.015 |
| dependent protein C | | | | |
| (Protein C) | | | | |
| Antithrombin III | P01008 | Coag | SERPINC1 | 0.018 |
| Vitamin K- | P22891 | Coag | PROZ | 0.021 |
| dependent protein Z | | | | |
| (Protein Z) | | | | |
| Coagulation factor | P12259 | Coag, canc signal | F5 | 0.022 |
| V | | | | |
| Apolipoprotein D | P05090 | Infl | APOD | 0.026 |
| Coagulation factor | P03951 | Coag | F11 | 0.026 |
| XI | | | | |
| Insulin-like growth | P17936 | Infl, canc signal | IGFBP3 | 0.027 |
| factor-binding | | | | |
| protein 3 (IBP 3) | | | | |
| L-selectin | P14151 | Infl | SELL | 0.035 |
| Plasma protease C1 | P05155 | Coag, complement | SERPING1 | 0.043 |
| inhibitor (C1 | | pathways, cell adhesion | | |
| inhibitor) | | | | |
| Plasma serine | P05154 | Coag, cell adhesion | SERPINA5 | 0.044 |
| protease inhibitor | | | | |
| (Protein C inhibitor) | | | | |
| ELISA measured | | | | |
| Guanylate cyclase A | P16066 | AF | NPR1 | 0.046 |
| (NPR1) (ANPR1) | | | | |
| Epidermal growth | P00533 | Infl | EGFR | 0.055 |
| factor receptor | | | | |
| (EGFR) | | | | |
| Glutamate receptor | Q12879 | Neurovasc unit inj | GRIN2A | 0.059 |
| ionotropic (NMDA | | | | |
| 2A or GRIN2A) | | | | |
| Fatty acid binding | P05413 | Neurovasc unit inj | FABP3 | 0.008 |
| protein 3 (FABP3) | | | | |
| Interleukin 6 (IL-6) | P05231 | Infl, canc signal | IL6 | 0.002 |
| S100A12 | P80511 | Infl | S100A12 | 0.002 |
| Enriched MRM measured | | | | |
| S100A12 | P80511 | Infl | | 0.009 |
| Epidermal growth | P00533 | Infl | | 0.010 |
| factor receptor | | | | |
| (EGFR) | | | | |
| Platelet endothelial | P16284 | | | 0.044 |
| cell adhesion | | Infl | | |
| molecule (PECAM1) | | | | |
| Prolactin | P01236 | Hormone | | 0.045 |

Marker type: Coag = coagulation, AF = atrial fibrillation, Infl = inflammation, Canc signal = cancer signaling, and neurovasc unit inj = neurovascular unit injury.
Protein Symbol reflects the terminology presented in FIG. 4.

TABLE 3
Antibodies used

| ELISA | Vendor | Product no. |
|---|---|---|
| Calcitonin (specifically Procalcitonin) | abcam | ab100630 |
| Dickkopf-related protein 1 | abcam | ab100501 |
| Epidermal growth factor receptor | abcam | ab100505 |
| (EGFR) | | |
| Heat shock protein beta-1 | abcam | ab113334 |
| Metalloproteinase inhibitor 4 | abcam | ab113328 |
| Myeloperoxidase | abcam | ab119605 |
| P-selectin | abcam | ab100631 |
| Stromelysin-1 | abcam | ab189572 |
| Tissue-type plasminogen activator | abcam | ab108914 |
| Tumor necrosis factor receptor | abcam | ab100643 |
| superfamily member 1B | | |
| Natriuretic peptides B (BNP) | Abnova | KA1861 |
| Protein S100-B | Antibodies | ABIN1117015 |
| | online | |
| Prolactin | Cayman | 500730 |
| | Chemical | |
| Guanylate cyclase A (NPR1) | LSBio | LS-F10832 |
| (ANPR1) | | |
| High mobility group protein B1 | LSBio | LS-F4038 |
| Protein S100-A12 | MBL | CY-8058 |
| Claudin-5 | mybiosource | MBS2024302 |
| Fatty acid binding protein 3 | mybiosource | MBS2020985 |
| (FABP3) | | |
| Glutamate receptor ionotropic | mybiosource | MBS2021633 |
| (NMDA 2A or GRIN2A) | | |
| Glutathione S-transferase P | mybiosource | MBS267722 |
| Microtubule-associated protein tau | mybiosource | MBS723516 |
| Myelin basic protein | mybiosource | MBS700083 |
| Neutrophil collagenase | mybiosource | MBS702847 |
| Nucleoside diphosphate kinase A | mybiosource | MBS900914 |
| Proenkephalin-A | mybiosource | MBS931043 |
| Vascular endothelial | mybiosource | MBS355262 |
| growth factor B | | |
| Eotaxin | R&D systems | DTX00 |
| Gamma-enolase | R&D systems | DENL20 |
| Interleukin 6 (IL-6) | R&D systems | D6050 |
| Lp-PLA | R&D systems | DPLG70 |
| L-selectin | R&D systems | BBE4B |
| Platelet endothelial cell | RayBiotech | ELH- |
| adhesion molecule | | PECAM1-1 |
| (PECAM) | | |

### REFERENCES

1. Amort et al., Transient Ischemic Attack versus Transient Ischemic Attack Mimics: Frequency, Clinical Characteristics and Outcome. 2011; 32:57-64. Available from: http://www.karger.com/doi/10.1159/000327034
2. Reynolds et al., Early biomarkers of stroke. Clinical chemistry. 2003; 49:1733-1739.
3. Lynch et al., Novel diagnostic test for acute stroke. Stroke; a journal of cerebral circulation. 2004; 35:57-63.
4. Allard et al., PARK7 and nucleoside diphosphate kinase A as plasma markers for the early diagnosis of stroke. Clinical chemistry. 2005; 51:2043-2051.
5. Jauch et al., Group FTNR-PSS. Association of Serial Biochemical Markers With Acute Ischemic Stroke. Stroke; a journal of cerebral circulation. 2006; 37:2508-2513.
6. Whiteley et al., Blood biomarkers for the diagnosis of acute cerebrovascular diseases: a prospective cohort study. Cerebrovascular diseases (Basel, Switzerland). 2011; 32:141-147.
7. Laskowitz et al., BRAIN Study Group. Clinical usefulness of a biomarker-based diagnostic test for acute stroke: the Biomarker Rapid Assessment in Ischemic Injury (BRAIN) study. Stroke; a journal of cerebral circulation. 2009; 40:77-85.
8. Sharma et al., A blood-based biomarker panel to detect acute stroke. Journal of stroke and cerebrovascular diseases: the official journal of National Stroke Association. 2014; 23:910-918.
9. Montaner et al., Etiologic Diagnosis of Ischemic Stroke Subtypes With Plasma Biomarkers. Stroke; a journal of cerebral circulation. 2008; 39:2280-2287.
10. Montaner et al., Differentiating ischemic from hemorrhagic stroke using plasma biomarkers: The S100B/RAGE pathway. Journal of proteomics. 2012; 75:4758-4765.
11. George P et al., Novel TIA biomarkers identified by mass spectrometry-based proteomics. International journal of stroke: official journal of the International Stroke Society. 2015; 10:n/a-n/a.
12. Sharma et al., Proteomic Signature of Endothelial Dysfunction Identified in the Serum of Acute Ischemic Stroke Patients by the iTRAQ-Based LC-MS Approach. J. Proteome Res. 2015; 14:2466-2479.
13. Knauer et al., A biochemical marker panel in MRI-proven hyperacute ischemic stroke-a prospective study. BMC Neurol. 2012; 12:14.
14. Penn A M W, Coutts S B, Borchers C H. Genome SpecTRA Project [Internet]. www.viha.ca. [cited 2017 Jan. 23]; Available from: http://www.viha.ca/rnd/current/spectra.htm
15. Nedelkov et al., Investigating diversity in human plasma proteins. Proceedings of the National Academy of Sciences of the United States of America. 2005; 102:10852-10857.
16. Penn et al., Group TSS. Exploring phlebotomy technique as a pre-analytical factor in proteomic analyses by mass spectrometryl. Genome. 2015; 58:569-576.
17. Adams et al., Classification of subtype of acute ischemic stroke. Definitions for use in a multicenter clinical trial. TOAST. Trial of Org 10172 in Acute Stroke Treatment. Stroke; a journal of cerebral circulation. 1993; 24:35-41.
18. Addona et al., Multi-site assessment of the precision and reproducibility of multiple reaction monitoring-based measurements of proteins in plasma. Nat. Biotechnol. 2009; 27:633-641.
19. Domanski et al., MRM-based multiplexed quantitation of 67 putative cardiovascular disease biomarkers in human plasma. Proteomics. 2012; 12:1222-1243.
20. Rousseeuw et al., robustbase: Basic Robust Statistics. R package version 0.92-3. 2015; 15:90-104.
21. Hastie T, Tibshirani R, Friedman J. The Elements of Statistical Learning; Data Mining, Inference and Prediction. 2008 ed. Springer, New York; 2008.
22. RCore T. R: A language and environment for statistical computing. R Foundation for Statistical Computing, Vienna, Austria. Online: http://www.R-project. org; 2013.
23. Szklarczyk et al., STRING v10: protein-protein interaction networks, integrated over the tree of life. Nucl. Acids Res. 2014; 43:gku1003-D452.
24. MacLean et al., Skyline: an open source document editor for creating and analyzing targeted proteomics experiments. Bioinformatics. 2010; 26:966-968.

### Example 3

### Methods

This example describes the methods used to generate the results described in Examples 4 and 9.

Drawing Blood

For the first study arm, blood was drawn at 3 time points. Draw 1 occurred in the emergency department (ED) within 6 hours of symptom onset. Draw 2 occurred 4-6 hours after the first blood draw. Draw 3 occurred 20-32 hours after the first blood draw. The second study arm (single blood draw) consisted of only one blood draw. This blood draw occurred within 24 hours of symptom onset. Enrollment into the study arms was based entirely upon patients' time of arrival at the ED after symptom onset (i.e., patients arriving at the ED later than 6 hours from symptom onset were enrolled in the single blood draw arm).

Plasma Preparation

Peripheral blood was drawn from the inside of the arm, as per standard of care at each study site. Blood was collected into 6.0 ml EDTA vacutainer tubes and immediately placed into an ice bath until processed; samples were processed within two hours of collection. The EDTA tubes were inverted 8-10 times before being centrifuged for 10-15 minutes at 2500-3000 rpm. Plasma samples were then pipetted into 500 μl aliquot tubes before being frozen at −80° C.

Diagnosis

Study participants received full neurological assessments, as per standard of care at each enrolling site. Study neurologists adjudicated cases on the basis of neurological assessments and radiological findings (i.e., MRI and CTA). Adjudicated diagnoses consisted of three levels: (a) mimic (negative imaging results); (b) ACVS possible (clinical presentation consistent with ACVS, but negative imaging result); and (c) ACVS definite (positive imaging results—DWI positive or abnormal CTA results). For analysis purposes, a binary diagnosis was derived by collapsing the ACVS possible and definitive diagnoses (0=Mimic, 1=ACVS).

Proteomic Analysis

Proteomic analysis was conducted using multiple reaction monitoring-mass spectrometry (MRM-MS) utilizing stable-isotope-labeled standard (SIS) peptides. The use of SIS peptides in MRM analyses is considered the “gold standard” in MS quantitation. (1,2)

Plasma samples were analyzed. Before MRM-MS analysis, the samples were randomly distributed across the analytic 96-well plates to control for potential batch effects. Samples were randomized on the basis of participant diagnosis, enrollment study site, and sex. The analyzer was blinded to participants' final diagnosis at the time of plasma analysis.

Candidate Proteins

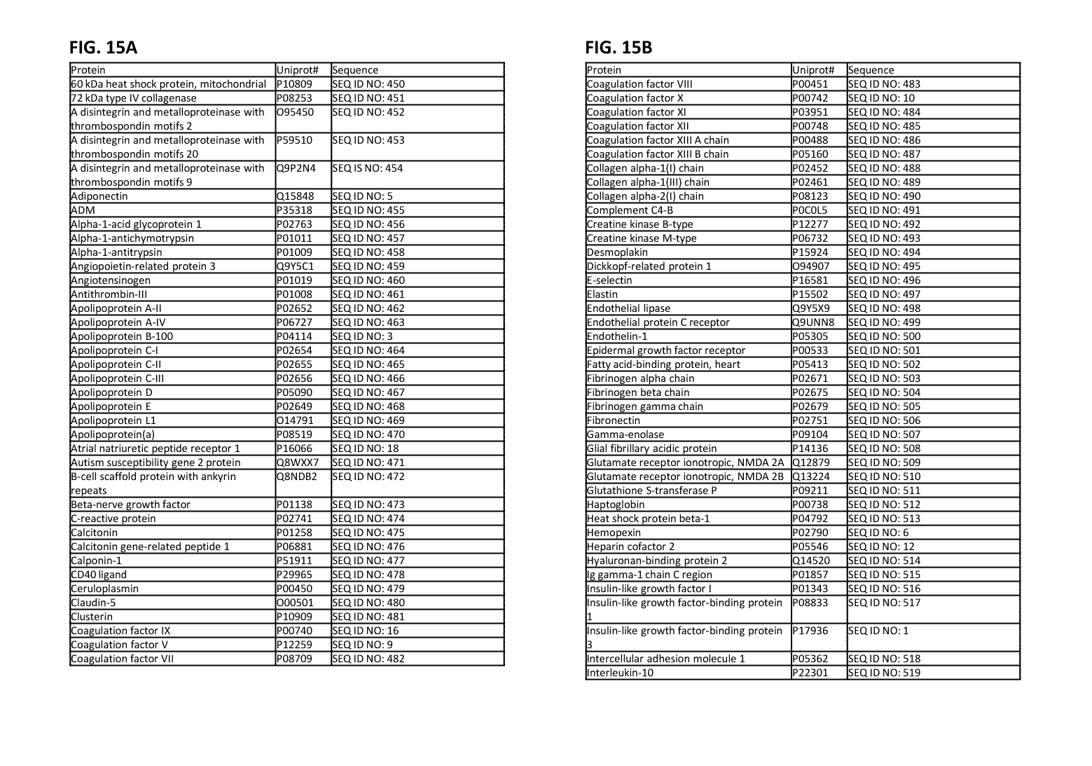

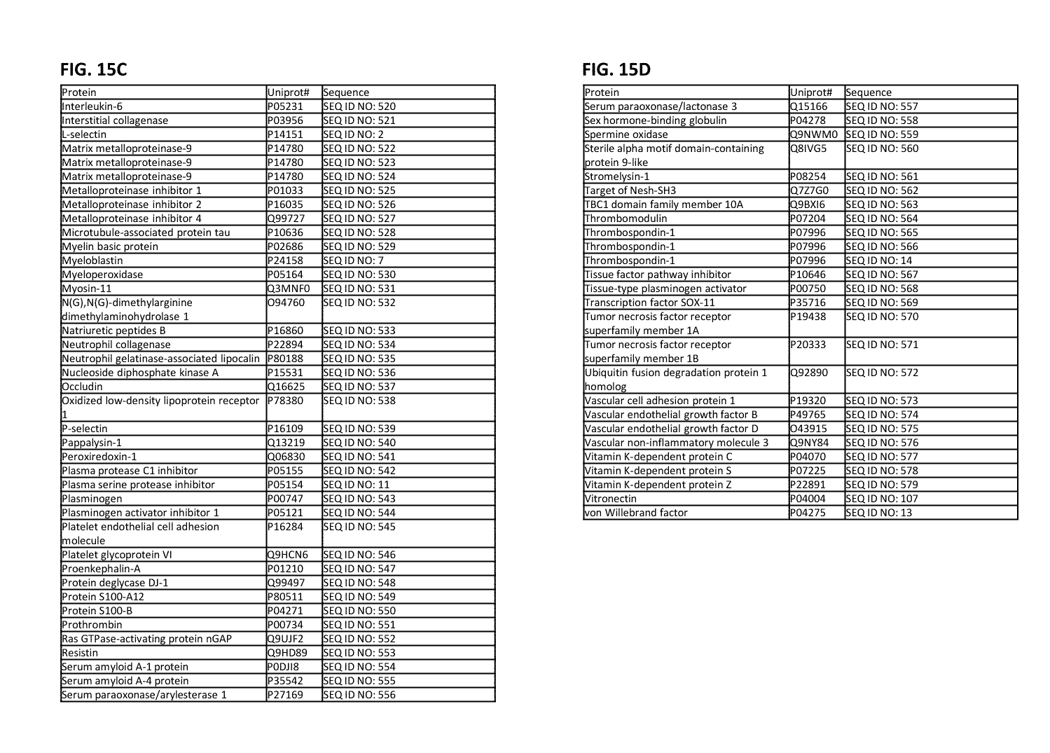

Candidate proteins to be examined were selected on the basis of a literature review of previously investigated protein biomarkers for TIA/mild stroke (see FIGS. 15A-15D). A total of 141 candidate proteins were examined. Each candidate protein was represented by a single peptide sequence for MRM analysis, with the exceptions of matrix metalloproteinase-9 (MMP-9) and thrombospondin-1 (TSP-1), which were measured using 3 distinct peptide sequences each, for a total of 145 candidate peptide sequences.

Participants

Inclusion criteria for enrollment were: (a) patients referred with suspected TIA or mild stroke (NIHSS<4), (b) symptom onset <24 hours, and (c) age ≥18 years of age. Exclusion criteria were: (a) isolated monocular blindness and (b) inability to obtain either MRI (within 7 days) or CT/CTA (within 24 hours). Symptom onset was defined as the last known time the patient was normal. Patients received either MRI or CTA imaging as part of the protocol.

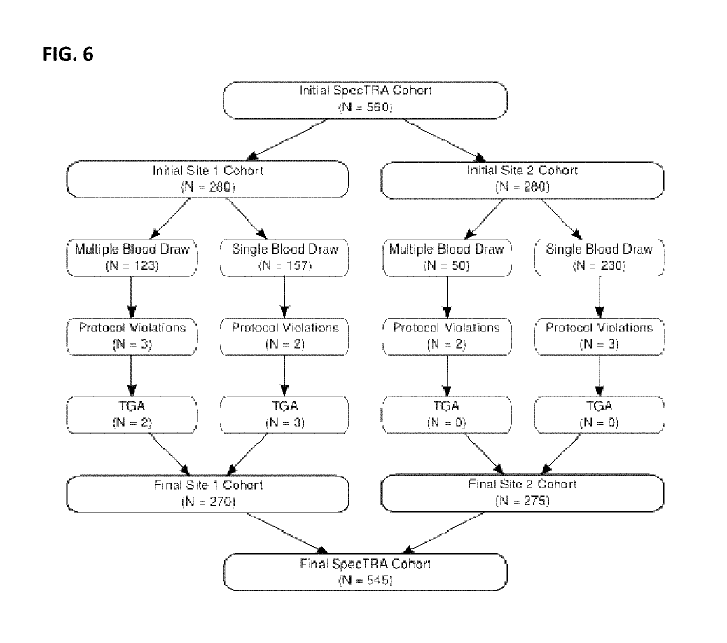

For the first phase of SpecTRA, patients were enrolled by stroke study nurses in the emergency departments (ED) of two urban medical hospitals. Stroke nurses recorded patients presenting clinical symptoms in the case report form (CRF) while patients were still in the ED. A total of 560 participants were enrolled in the first phase. Of the initial sample, 10 patients were removed due to protocol violations, such as missing necessary brain imaging. Due to medical and clinical ambiguity regarding Transient Global Amnesia (TGA; 3) and its potential relation to ACVS, an additional 5 patients were removed from the sample. In total, 15 patients were excluded from the dataset (FIG. 6). TABLE 4 displays the demographic characteristics of the SpecTRA dataset (N=545).

TABLE 4
Demographics

| | SpecTRA Study | | Site 1 | | Site 2 | | * |
|---|---|---|---|---|---|---|---|
| N | 545 | | 270 | | 275 | | |
| Patient Age, mean (sd) | 68.9 | (15.2) | 72.6 | (14.4) | 65.2 | (15.1) | <0.001 |
| Male, N (%) | 290 | (53.2) | 137 | (50.7) | 153 | (55.6) | 0.5191 |
| Diagnosis of ACVS, N (%) | 386 | (70.8) | 194 | (71.9) | 192 | (69.8) | 0.8725 |
| CTA Completed, N (%) | 440 | (80.7) | 192 | (71.1) | 248 | (90.2) | <0.001 |
| MRI Completed, N (%) | 522 | (95.8) | 268 | (99.3) | 254 | (92.4) | <0.001 |
| ABCD2, N (%) | | | | | | | |
| 0 | 1 | (0.2) | 1 | (0.4) | 0 | (0.0) | 0.6469 |
| 1 | 5 | (0.9) | 2 | (0.7) | 3 | (1.1) | |
| 2 | 29 | (5.3) | 18 | (6.7) | 11 | (4.0) | |
| 3 | 67 | (12.3) | 34 | (12.6) | 33 | (12.0) | |
| 4 | 122 | (22.4) | 64 | (23.7) | 58 | (21.1) | |
| 5 | 133 | (24.4) | 51 | (18.9) | 82 | (29.8) | |
| 6 | 166 | (30.5) | 87 | (32.2) | 79 | (28.7) | |
| 7 | 22 | (4.0) | 13 | (4.8) | 9 | (3.3) | |
| Systolic BP, mean (sd) | 156.2 | (27.2) | 158.0 | (26.8) | 154.4 | (27.5) | 0.3059 |
| Diastolic BP, mean (sd) | 83.7 | (14.1) | 83.3 | (13.7) | 84.1 | (14.5) | 0.8040 |
| Hypertension, N (%) | 313 | (57.4) | 167 | (61.9) | 146 | (53.1) | 0.1178 |
| Hyperlipidaemia, N (%) | 217 | (39.8) | 107 | (39.6) | 110 | (40.0) | 0.9961 |
| Atrial Fibrillation, N (%) | 67 | (12.3) | 38 | (14.1) | 29 | (10.5) | 0.4554 |
| Diabetes, N (%) | 92 | (16.9) | 40 | (14.8) | 52 | (18.9) | 0.4432 |
| Smoking, N (%) | 54 | (9.9) | 18 | (6.7) | 36 | (13.1) | 0.0429 |

For participants, a motor/speech deficit variable (0=absent, 1=present) was also constructed using information from the CRF and was defined as the presence of any of the following clinical symptoms: (a) face droop, (b) unilateral limb weakness, (c) speech deficit, and (d) language disturbance.

Quality Control

Prior to statistical analysis, proteomic data are examined for quality control (QC). Analysis is conducted on the relative ratios of the areas of the natural (NAT) and stable-isotope-labeled standard (SIS) peptides for each peptide. QC occurs in several steps and is blinded to a participant's final diagnosis.

In the first step of QC, peptides in which over 75% of the samples have measurement values below the limit of quantification are removed from the list of candidate proteins. In the second step, peptides in which 80% or greater of the observations occurred in the lowest 10 values of the peptide are removed.

Before completing the next phase of QC (review), the data are examined for possible time effects on peptide expression values. Thereafter, the proteomic results are manually reviewed for each peptide and each participant to ensure that all proteomic values are above the limits of detection to avoid integration of non-specific interfering compounds or random noise. Peptide data are flagged as either valid or suspect.

In the final step of QC, for each peptide, the proportion of valid values out of the total number of participants is calculated. Peptides with less than 20% valid values are removed from the candidate peptide list. The proportions of valid peptide values are retained for later analyses.

Proteomic Data Imputation and Transformation

For peptides with ratio to standard values of zero (i.e., a value below the limit of quantification), values are imputed using half of the value of the minimum for each peptide. Peptide values are log 2 transformed and standardized (mean/sd) before statistical analyzes are c onducted.

Time Effect on Proteomic Expression

To evaluate for any possible interactions between diagnosis and blood draw time (relative to symptoms onset) on peptide expression, three successive effect analyses are used.

In the first effect analysis, a linear mixed-effects model that maximized the log-likelihood is fit that regresses each peptide on: (a) time (i.e., time between symptom onset and blood draw time); (b) time squared (i.e., timet); (c) group (i.e., diagnostic group, 3 levels); (d) analytic plate ID; (e) interaction between time:group; and (f) interaction between time²:group, with the participant study ID as a random effect. The squared value for time will be included in the model to allow for curvature of the trend line for peptide expression. This full model is then compared to a restricted model fit without the time²:group interaction using ANOVA. The p-values of the resulting likelihood tests are adjusted for multiple comparisons using a false discovery rate (FDR)4 of α=0.2.

In the second time effect analysis, the previous procedure is repeated with the time²:group interaction term removed from the full model, and the time:group term removed restricted models. This analysis, therefore, examines the time:group interaction effect on peptide expression.

In the third time effect analysis, the models from the second analysis are further reduced by removing the time:group interaction term from the full model and the time² term from the restricted model. This analysis evaluates non-linear relationships (i.e., time²) between time and peptide expression.

Correcting for Batch Effects

After the data are examined for potential interactions between blood draw time and diagnosis, the data are corrected for batch effects. For each peptide, relative ratio values are regressed on (a) time and (b) analytic plate ID using linear regression. In the event that substantial interactions between time and diagnostic group are observed, these terms are added to the linear regression model as needed. The resulting normal residuals will constitute the final proteomic values for statistical analysis.

Univariate Analyses

After the proteomic data are corrected for batch and time effects, univariate analyzes are conducted for each peptide. Using robust generalized linear models (5), the binary diagnosis variable are regressed for each individual candidate peptide as the sole predictor. Odds ratios with 95% confidence intervals are calculated for each peptide; p-values for the peptides are adjusted using an FDR of α=0.2.

After descriptive univariate analyses are conducted, a proteomic biomarker panel is constructed using Lasso logistic regression (6) in conjunction with bootstrapping (7); the procedure is comparable to that proposed by Wang et al. (8) The panel will be constructed as follows.

Using the entire first phase SpecTRA study sample (N=545), 300 bootstrap samples (sampled with replacement) are created. Bootstrap samples are stratified by diagnostic level (i.e., mimic, ACVS possible, and ACVS definitive) and the presence of motor/speech deficits.

For each bootstrap sample, a Lasso logistic regression model, restricted to have up to a maximum of 15 predictors, is used to regress the binary version of the diagnosis variable for the candidate peptides. The Lasso model employs a penalty factor, where the penalty for each candidate peptide's inclusion in the model is directly proportional to the proportion of valid peptide values as determined by the PC's manual QC review. For each of 300 Lasso models, the predictors with non-zero coefficients are recorded to an ongoing tally. The tallies for the peptides are then sorted in descending order, and peptides that appear in at least one-third of the trials (N≥100) are included in the proteomic panel.

Preliminary analyses are conducted to assess the discriminant predictive performance of the constructed biomarker panel. A penalized logistic regression model, utilizing the entire study sample, is used to regress the binary diagnosis for the panel peptides; the previously described penalty factor is used during model fitting. The model is evaluated using repeated 100×10-fold cross-validation (CV). Performance is evaluated on both the entire sample and the subset of participants who are motor/speech negative. Optimism-corrected ROC curves and area under the ROC curves (AUC) are generated.

The analyses were completed using the ROCR (v1.0.7; 9), pROC (v1.9.1; 10) glmnet (v2.0.5; 6), robustbase (v0.92.7; 5), nmle (v3.1.128; 11), Hmisc (v4.0.2; 12), rms (v5.1.0; 13), and ggplot2 (v2.2.1; 14) libraries in the R statistical language (v3.3.2; 15).

### Example 4

### Results

Quality Control—Phase 1

In the first stage of QC, 2 peptides (one MMP-9 sequence and Creatine kinase B-type) were removed from the candidate list for having ≥75% of values below the limit of quantification. In the second step of QC, 36 peptides were removed from the candidate list for having ≥80% of the observations in the lowest 10 values of the peptide. A total of 107 peptides of the initial 145 candidate peptides passed the first two steps of QC.

Time Effect on Proteomic Expression

Examination of the FDR-adjusted p-values for the likelihood tests of the time²:group interaction term did not indicate significant effects of the interaction on peptide expression (α=0.2). For the time:group interaction analysis, three peptides were found to have significant interactions: (a) apolipoprotein A-IV, (b) B-cell scaffold protein with ankyrin repeats, and (c) collagen alpha-1(I) chain. The timet interaction term was non-significant.

Adjustment for Time and Plate Effects

For further analyses, the data set was restricted to only participants' first blood draw to render the data i.i.d (i.e., removing blood draws 2 and 3 for patients with multiple blood draws). QC steps 1 and 2 were repeated on the peptide values for the first blood draw (N=545 participants). A total of 105 peptides passed the first two QC steps for the first blood draw.

Quality Control—Phase 2

After the 105 peptides passing the first two phases of QC for the first blood draw were reviewed (i.e., 545 participants×105 peptides=57225 individual peptide datum), 46 peptides were removed from the candidate list for having <20% valid proteomic values.

A total of 59 peptides (corresponding to 59 distinct proteins) remained in the candidate peptide list after QC was completed.

Univariate Analysis of First Blood Draw

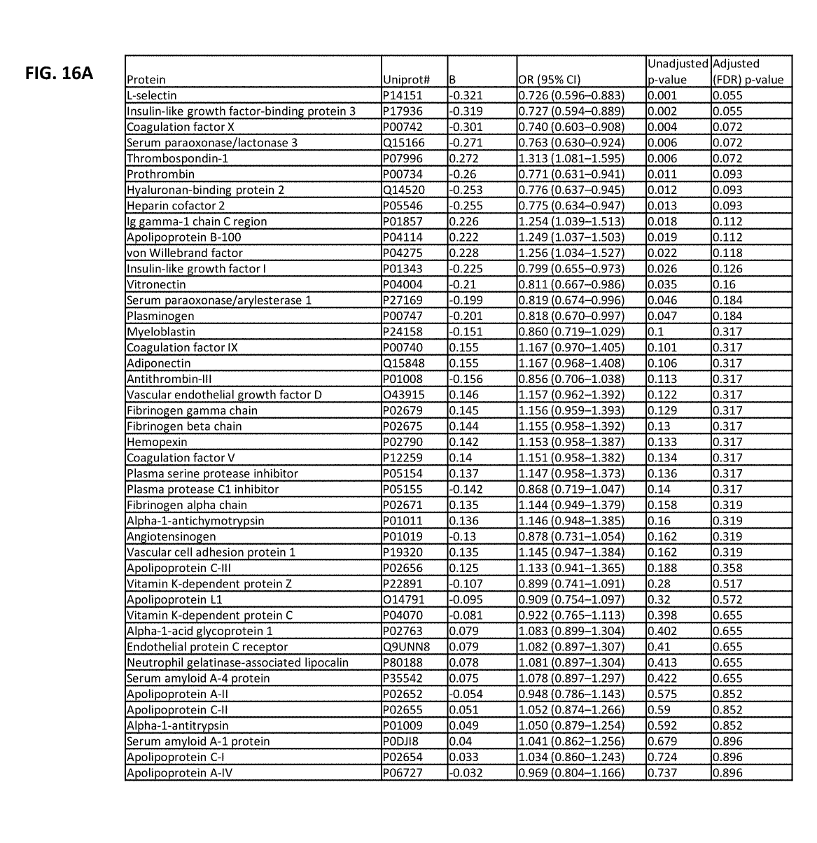

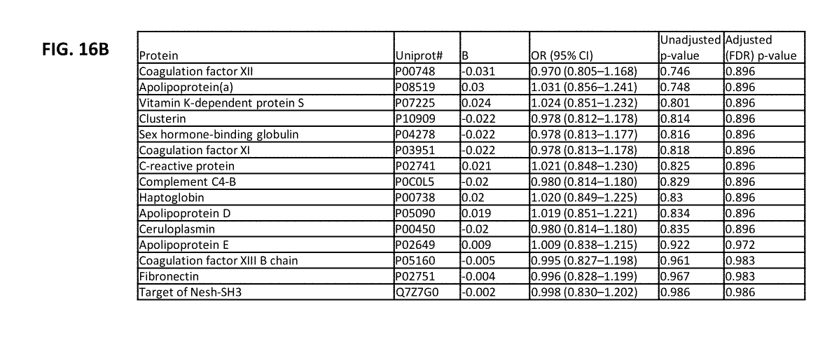

Robust generalized linear models predicting the binary diagnosis (0=Mimic; 1=ACVS) were fit to each peptide. FIGS. 16A-16B display the results of the model fits (N=59 peptides) with FDR-adjusted p-values. Fifteen peptides were differentiated between ACVS and mimic patients after FDR adjustment (α=0.2).

Construction of a Proteomic Panel

TABLE 5 displays the final proteomic panel (N=16 peptides) with frequency of selection by the bootstrapping procedure, as previously described.

TABLE 5
Peptides selected by bootstrap Lasso procedure
with frequency of peptide selection across
bootstrap samples (N = 300).

| | | Frequency |
|---|---|---|
| | Peptide | of |
| Protein | Sequence | Selection |
| Insulin-like growth | FLNVLSPR | 300 |
| factor-binding | (SEQ ID NO: 1) | |
| protein 3 | | |
| L-selectin | AEIEYLEK | 237 |
| | (SEQ ID NO: 2) | |
| Apolipoprotein B-100 | FPEVDVLTK | 212 |
| | (SEQ ID NO: 3) | |
| Vascular endothelial | DLIQHPK | 200 |
| growth factor D | (SEQ ID NO: 4) | |
| Adiponectin | GDPGLIGPK | 193 |
| | (SEQ ID NO: 5) | |
| Hemopexin | NFPSPVDAAFR | 188 |
| | (SEQ ID NO: 6) | |
| Myeloblastin | LVNVVLGAHNVR | 180 |
| | (SEQ ID NO: 7) | |
| Serum paraoxonase/ | ILIGTVFHK | 178 |
| lactonase 3 | (SEQ ID NO: 8) | |
| Coagulation factor V | AEVDDVIQVR | 173 |
| | (SEQ ID NO: 9) | |
| Coagulation factor X | TGIVSGFGR | 171 |
| | (SEQ ID NO: 10) | |
| Plasma serine protease | AAAATGTIFTFR | 170 |
| inhibitor | (SEQ ID NO: 11) | |
| Heparin cofactor 2 | TLEAQLTPR | 139 |
| | (SEQ ID NO: 12) | |
| von Willebrand factor | ILAGPAGDSNVVK | 132 |
| | (SEQ ID NO: 13) | |
| Thrombospondin-1 | GPDPSSPAFR | 130 |
| | (SEQ ID NO: 14) | |
| Hyaluronan-binding | DEIPHNDIALLK | 128 |
| protein 2 | (SEQ ID NO: 15) | |
| Coagulation factor IX | SALVLQYLR | 120 |
| | (SEQ ID NO: 16) | |

The panel included 9 of the 15 peptides with a significant univariate relation to the binary diagnosis outcome (ACVS vs. mimic): (a) L-selectin, (b) insulin-like growth factor-binding protein 3 (IGFBP-3), (c) coagulation factor X (F10), (d) serum paraoxonase/lactonase 3 (PON3), (e) thrombospondin-1 (TSP-1), (f) hyaluronan-binding protein 2 (HABP2), (g) heparin cofactor 2 (HCII), (h) apolipoprotein B-100 (apoB-100), and (i) von Willebrand factor (vWF).

Proteomic Panel Performance

To assess the performance of the final proteomic panel, a penalized logistic regression model was fit to the data. TABLE 6 displays the coefficients of the model.

TABLE 6
Penalized logistic regression model fit to
biomarker panel after standardization
(mean/sd) of peptide values.

| | Peptide | Uniprot | |
|---|---|---|---|
| Protein | Sequence | # | B |
| (Intercept) | | | 0.985 |
| Plasma serine | AAAATGTIFTFR | P05154 | 0.15 |
| protease | (SEQ ID NO: 11) | | |
| inhibitor | | | |
| L-selectin | AEIEYLEK | P14151 | −0.233 |
| | (SEQ ID NO: 2) | | |
| Coagulation factor | AEVDDVIQVR | P12259 | 0.171 |
| V | (SEQ ID NO: 9) | | |
| Hyaluronan-binding | DEIPHNDIALLK | Q14520 | −0.203 |
| protein 2 | (SEQ ID NO: 15) | | |
| Vascular | DLIQHPK | O43915 | 0.241 |
| endothelial | (SEQ ID NO: 4) | | |
| growth factor D | | | |
| Insulin-like growth | FLNVLSPR | P17936 | −0.048 |
| factor-binding | (SEQ ID NO: 1) | | |
| protein 3 | | | |
| Apolipoprotein | FPEVDVLTK | P04114 | 0.145 |
| B-100 | (SEQ ID NO: 3) | | |
| Adiponectin | GDPGLIGPK | Q15848 | 0.255 |
| | (SEQ ID NO: 5) | | |
| Thrombospondin-1 | GPDPSSPAFR | P07996 | 0.155 |
| | (SEQ ID NO: 14) | | |
| von Willebrand | ILAGPAGDSNVVK | P04275 | 0.116 |
| factor | (SEQ ID NO: 13) | | |
| Serum paraoxonase/ | ILIGTVFHK | Q15166 | −0.207 |
| lactonase 3 | (SEQ ID NO: 8) | | |
| Myeloblastin | LVNVVLGAHNVR | P24158 | −0.183 |
| | (SEQ ID NO: 7) | | |
| Hemopexin | NFPSPVDAAFR | P02790 | 0.208 |
| | (SEQ ID NO: 6) | | |
| Coagulation factor | SALVLQYLR | P00740 | 0.283 |
| IX | (SEQ ID NO: 16) | | |
| Coagulation factor | TGIVSGFGR | P00742 | −0.231 |
| X | (SEQ ID NO: 10) | | |
| Heparin cofactor 2 | TLEAQLTPR | P05546 | −0.262 |
| | (SEQ ID NO: 12) | | |

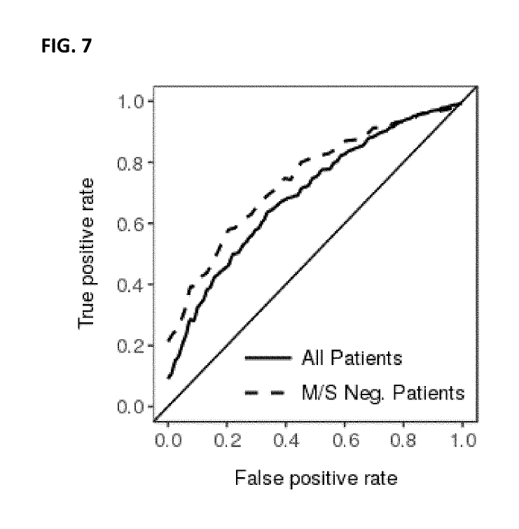

FIG. 7 displays optimism corrected ROC curves of the penalized logistic regression model. The model had an optimism correct AUC of 0.699 when applied to all the participants in the sample (N=545). When applied to only the subset of patients who were motor/speech negative (N=130), the model achieved an optimism correct AUC of 0.764.

Coagulation factor V was predictive of ACVS (16,17). Apolipoprotein B-100 was predictive of ACVS, which is in keeping with some previously published results (18,19) but not others (20,21). Adiponectin was predictive of ACVS, which is in keeping with some previously published results (22,23) but not others (24-27). Thrombospondin-1 was predictive of ACVS, which is in keeping with some previously published results (28,29) but not others (30). Von Willebrand factor was predictive of ACVS (31-34). Coagulation factor IX was predictive of ACVS (16). Coagulation factor X was predictive of ACVS, which is not in keeping with previously published results (16,17).

### REFERENCES

1. Ong S E, Mann M. Mass spectrometry-based proteomics turns quantitative. Nat Chem Biol 2005; 1: 252-262.
2. Parker C E, Borchers C H. Mass spectrometry based biomarker discovery, verification, and validation—quality assurance and control of protein biomarker assays. Mol Oncol 2014; 8: 840-858.
3. Sedlaczek et al., Detection of delayed focal M R changes in the lateral hippocampus in transient global amnesia. Neurology 2004; 62: 2165-2170.
4. Benjamini Y, Hochberg Y. Controlling the false discovery rate: a practical and powerful approach to multiple testing. J R Stat Soc Series B Stat Methodol 1995; 57: 289-300.
5. Maechler et al., robustbase: Basic robust statistics, http://CRAN.R-project.org/package=robustbase (2016).
6. Friedman et al., Regularization paths for generalized linear models via coordinate descent. J Stat Softw 2010; 33: 1-22.
7. Efron B, Tibshirani R. Bootstrap methods for standard errors, confidence intervals, and other measures of statistical accuracy. Statistical Science 1986; 1: 54-75.
8. Wang S, Nan B, Rosset S, et al. Random lasso. Ann Appl Stat 2011; 5: 468-485.
9. Sing et al., ROCR: visualizing classifier performance in R. Bioinformatics 2005; 21: 3940-3941.
10. Robin et al., pROC: an open-source package for R and S+ to analyze and compare ROC curves. BMC Bioinformatics 2011; 12: 77.
11. Pinheiro et al., nlme: Linear and nonlinear mixed effects models, https://CRAN.R-project.org/package=nlme (2016).
12. Harrell Jr F E, Dupont C. Hmisc: Harrell miscellaneous, https://CRAN.R-project.org/package=Hmisc (2016).
13. Harrell Jr F E. rms: Regression modeling strategies, https://CRAN.R-project.org/package=rms (2016).
14. Wickham H. ggplot2: Elegant graphics for data analysis. New York, N.Y.: Springer-Verlag, 2009.
15. R Core Team. R: A language and environment for statistical computing. Vienna, Austria: R Foundation for Statistical Computing, https://www.R-project.org (2016).
16. Gissel et al., Plasma factor and inhibitor composition contributes to thrombin generation dynamics in patients with acute or previous cerebrovascular events. Thromb Res 2010; 126: 262-269.
17. Biasiutti et al., al. Hemostatic risk factors in ischemic stroke. Thromb Haemost 2003; 90: 1094-1099.
18. Shilpasree et al., A study of serum apolipoprotein al, apolipoprotein b and lipid profile in stroke. J Clin Diagn Res 2013; 7: 1303-1306.
19. Agapakis et al., Mo-p4:282 apolipoprotein a-1, apolipoprotein b and lipoprotein (a) levels at patients with ischemic stroke. Atherosclerosis Supplements 2006; 7: 108.
20. Lopez et al., Discrimination of ischemic and hemorrhagic strokes using a multiplexed, mass spectrometry-based assay for serum apolipoproteins coupled to multi-marker ROC algorithm. Proteomics Clin Appl 2012; 6: 190-200.
21. Sharobeem et al., Elevated lipoprotein (a) and apolipoprotein b to AI ratio in south asian patients with ischaemic stroke. Int J Clin Pract 2007; 61: 1824-1828.
22. Lukasik et al., Reactive leptin resistance and the profile of platelet activation in acute ischaemic stroke patients. Thromb Haemost 2012; 108: 107-118.
23. Prugger et al., Adipocytokines and the risk of ischemic stroke: the PRIME study. Ann Neurol 2012; 71: 478-486.
24. Marousi et al., erum adiponectin acutely after an ischemic stroke: implications for a long-lasting, suppressed anti-inflammatory role. Acta Neurol Scand 2010; 121: 277-284.
25. Kim et al., Adipocytokines and ischemic stroke: differential associations between stroke subtypes. J Neurol Sci 2012; 312: 117-122.
26. Pera et al., Temporal changes of adiponectin plasma levels in patients with acute ischemic stroke. Neurol Res 2013; 35: 988-991.
27. Efstathiou et al., Plasma adiponectin levels and five-year survival after first-ever ischemic stroke. Stroke 2005; 36: 1915-1919.
28. Gao et al., Predictive value of thrombospondin-1 for outcomes in patients with acute ischemic stroke. Clin Chim Acta 2015; 450: 176-180.
29. Dong et al., Changes in plasma thrombospondin-1 concentrations following acute intracerebral hemorrhage. Clin Chim Acta 2015; 450: 349-355.
30. Navarro-Sobrino et al., A large screening of angiogenesis biomarkers and their association with neurological outcome after ischemic stroke. Atherosclerosis 2011; 216: 205-211.
31. Kozuka et al., Endothelial markers and adhesion molecules in acute ischemic stroke—sequential change and differences in stroke subtype. Atherosclerosis 2002; 161: 161-168.
32. Kraft et al., Von Willebrand factor regulation in patients with acute and chronic cerebrovascular disease: a pilot, case-control study. PLOS ONE 2014; 9: e99851.
33. Reynolds M A. Early biomarkers of stroke. Clin Chem 2003; 49: 1733-1739.
34. Lynch J R. Novel diagnostic test for acute stroke. Stroke 2003; 35: 57-63.

### Example 5

### iMALDI Procedure

For plasma digestion, which is automated using Tecan Freedom Evo, 9 M urea, 300 mM Tris, and 20 mM DTT, pH 8, is added to human plasma. Thereafter, 100 mM iodoacetamide, 100 mM Tris, and TPCK-treated trypsin in 100 mM Tris is added. The mixture is incubated at 37° C. for 18 hrs. Thereafter, 10% formic acid is added.

To prepare beads, which is automated using Agilent Bravo, Protein G Dynabeads are transferred to a 96-well plate. The beads are washed 7 times with 25% ACN in PBSC. The beads are then washed 3 times with PBSC. Purified anti-peptide pAb is added to the washed beads, and the beads are incubated for 1 hr at room temperature with agitation. The protein G Dynabead/anti-peptide pAb complexes are then washed with PBSC, resuspended in PBSC, and then stored on ice until used. Where multiplex enrichment experiments are used, protein G Dynabead/anti-peptide pAb complexes are combined.

For peptide capture, which is automated using Agilent Bravo, the washed protein G Dynabead-pAb complexes are incubated with the human plasma digest for 1 hr at room temperature with agitation. The mixture is washed 2 times with 15% ACN in PBSC, one time with 15% ACN in 5 mM ammonium bicarbonate, and one time with 5 mM Ammonium bicarbonate. Next, the bound peptide is eluted from the protein G Dynabead-pAb complexes with 3 mg/mL HCCA in 70% ACN, 0.15% TFA. The bead/eluate mixture is spotted directly onto the MALDI plate.

For the MALDI experiment, HCCA matrix is placed on the spotted samples and allowed to dry. The sample-matrix spots are washed three times with 5 mM ammonium citrate, and the dried sample-matrix spots are analyzed using a Bruker Microflex LRF. FIG. 8 shows confirmed targets for iMALDI, and FIG. 9 shows additional iMALDI targets.

TABLE 7
Reagents used in iMALDI

| (A) Plasma | (B) Bead | (C) Peptide | |
|---|---|---|---|
| Digestion | Preparation | Capture | (D) MALDI |
| Human Plasma | Protein G | Human plasma | 5 mM |
| | Dynabeads | digest | Ammonium |
| | | | citrate |
| 9M Urea, | 25% ACN | 15% ACN | 3 mg/mL |
| 300 mM Tris, | in PBSC | in 5 mM | HCCA in |
| 20 mM DTT, | | Ammonium | 70% ACN, |
| pH 8 | | Bicarbonate | 0.15% TFA |
| 100 mM | PBSC | 15% ACN | |
| Iodoacetamide | | in PBSC | |
| 100 mM | | 5 mM | |
| TRIS, pH 8 | | Ammonium | |
| | | bicarbonate | |
| 1 mg/mL TPCK- | | | |
| treated trypsin in | | | |
| 100 mM TRIS | | | |
| 10% Formic acid | | | |

### Example 6

### Methods

Examples 8 and 9 provide an additional evaluation of the model performance for the biomarker panel and neurologist-adjudicated patient cases described in Examples 3 and 4. In addition to Example 3, this example describes the methods used to generate the results described in Example 9.

The primary logistic regression models are based on a locked-down panel of 15 peptides (+/−motor & speech: GLM-15 and GLM-15+M/S) that were fit to the data in Examples 3 and 4 (N=545, 386 ACVS vs. 159 Mimic) and were formally evaluated (N=575, 414 ACVS vs. 161 Mimic) against the previously established biomarker performance target scenarios. Similar analyses were performed with 5- and 4-peptide panels (see TABLE 8); these smaller panels focus on peptides that are used in the iMALDI platform described in Example 5.

The GLM-15 model significantly exceeded the target performance in Scenario A (replace M/S score for detection of ACVS), and GLM-15+M/S achieved the target performance in Scenario C (upgrade medical imaging urgency). Similar results were obtained in secondary analyses of other candidate models.

Biomarker Performance Targets

In the data from Examples 3 and 4 (N=545 patients), the motor/speech (M/S) rule (presence of motor weakness or language disturbance (aphasia) or speech disturbance (dysarthria)) showed a sensitivity of 0.801 and specificity of 0.333 for detecting ACVS versus Mimic. These data were used as performance targets in the following Scenarios.

Scenario A: replace M/S score for detection of ACVS. The proteomic biomarker score must produce a rule with specificity greater than 0.33 for sensitivity fixed at a minimum of 0.80.

Scenario B: upgrade Stroke Unit referral urgency for M/S-negative. The proteomic biomarker score must have a sensitivity of at least 0.5 for a specificity fixed at a minimum of 0.83 using a subset of patients who are M/S negative.

Scenario C: upgrade medical imaging urgency. The combination of the proteomic biomarker score with the M/S algorithm must improve the specificity of the M/S algorithm from 0.33 to 0.50 while maintaining the sensitivity at 0.80.

Evaluation

The target was achieved if the upper bound of the conventional 95% confidence interval (CI) for the sensitivity or specificity exceeded the target for the parameter of interest. When the lower bound of the 95% CI exceeded the target, the target was significantly exceeded.

Models

The models were selected and fit using the data from Examples 3 and 4, and the analyses were completed prior to examination of the unblinded data. The peptides used are a subset of the proteomic panel of N=16 peptides (TABLE 8).

GLM-15 is a simple logistic regression model that includes the 15 peptides available in the MRM data from among the original panel of 16 (i.e., excluding the peptide for thrombospondin-1, for which different peptides were used in the MRM assays of Examples 3 and 4).

GLM-16 is a simple logistic regression model that includes all 16 of the panel of peptides, imputing all thrombospondin-1 measurements to their mean value (effectively removing it from the model without adjusting the remaining coefficients).

GLM-4 is a simple logistic regression model that includes the 4 peptides available in the MRM data from among a panel of 6 used the iMALDI assay.

GLM-5 is a simple logistic regression model fit that includes the 4 peptides from GLM-4 with an additional peptide that is also used for iMALDI (von Willebrand Factor).

TABLE 8
Proteins and their corresponding peptides used
in the models. The proteins in Examples 8 and
9 were measured using these peptide sequences.

| | | GLM- | GLM- | GLM- | GLM- |
|---|---|---|---|---|---|
| Protein | Peptide Sequence | 15 | 16 | 5 | 4 |
| Insulin-like | FLNVLSPR | ✓ | ✓ | ✓ | ✓ |
| growth | (SEQ ID NO: 1) | | | | |
| factor-binding | | | | | |
| protein 3 | | | | | |
| L-selectin | AEIEYLEK | ✓ | ✓ | ✓ | ✓ |
| | (SEQ ID NO: 2) | | | | |
| Apolipoprotein | FPEVDVLTK | ✓ | ✓ | ✓ | ✓ |
| B-100 | (SEQ ID NO: 3) | | | | |
| Vascular | DLIQHPK | ✓ | ✓ | | |
| endothelial | (SEQ ID NO: 4) | | | | |
| growth factor D | | | | | |
| Adiponectin | GDPGLIGPK | ✓ | ✓ | | |
| | (SEQ ID NO: 5) | | | | |
| Hemopexin | NFPSPVDAAFR | ✓ | ✓ | | |
| | (SEQ ID NO: 6) | | | | |
| Myeloblastin | LVNVVLGAHNVR | ✓ | ✓ | | |
| (Neutrophil | (SEQ ID NO: 7) | | | | |
| Protease-4) | | | | | |
| Serum | ILIGTVFHK | ✓ | ✓ | | |
| paraoxonase/ | (SEQ ID NO: 8) | | | | |
| lactonase 3 | | | | | |
| (Paraoxonase- | | | | | |
| PON 3) | | | | | |
| Coagulation | AEVDDVIQVR | ✓ | ✓ | | |
| factor V | (SEQ ID NO: 9) | | | | |
| Coagulation | TGIVSGFGR | ✓ | ✓ | | |
| factor X | (SEQ ID NO: 10) | | | | |
| Plasma serine | AAAATGTIFTFR | ✓ | ✓ | | |
| protease | (SEQ ID NO: 11) | | | | |
| inhibitor | | | | | |
| (AKA Protein | | | | | |
| C inhibitor) | | | | | |
| Heparin | TLEAQLTPR | ✓ | ✓ | | |
| cofactor 2 | (SEQ ID NO: 12) | | | | |
| von Willebrand | ILAGPAGDSNVVK | ✓ | ✓ | ✓ | |
| Factor | (SEQ ID NO: 13) | | | | |
| Thrombospondin- | GPDPSSPAFR* | | * | | |
| 1 | (SEQ ID NO: 14) | | | | |
| Hyaluronan- | DEIPHNDIALLK | ✓ | ✓ | | |
| binding | (SEQ ID NO: 15) | | | | |
| protein 2 | | | | | |
| Coagulation | SALVLQYLR | ✓ | ✓ | ✓ | ✓ |
| factor IX | (SEQ ID NO: 16) | | | | |

*Thrombospondin-1 was represented by GTLLALER in Examples 3 and 4; for analysis in GLM-16, all values were set equal to their overall mean value, effectively removing thrombospondin-1 from the analysis without adjusting other coefficients.

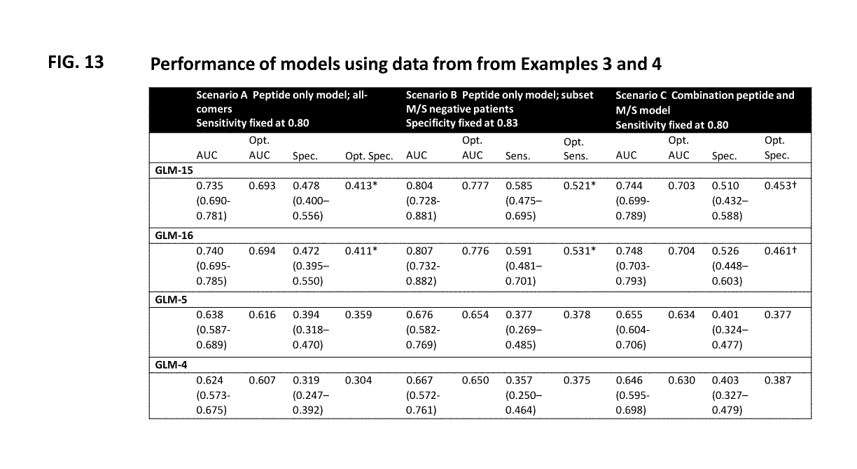

Each model was fit using logistic regression with the respective peptide panels plus a binary Motor/Speech variable. FIG. 13 and TABLES 9-12 provide model summaries for the peptide-only and peptide+M/S models fit in Examples 3 and 4. For reference, the analysis results for these models using the data from Examples 3 and 4 are presented in FIG. 13.

### Example 7

### Results

The analysis used the initial proteomic dataset, comprising 63 peptides for 59 proteins for 600 experimental samples from patients. Four of the proteins were measured with two peptides; the peptide used in Examples 3 and 4 with another peptide that better measured the corresponding proteins. To expand the models in Examples 3 and 4, the analysis was restricted to the peptides used therein.

Data Preparation and Quality Control

Without considering clinical information (i.e., uninformed by diagnosis) and based on quality control (QC), samples were removed that exhibited preparation and quantitation complications as identified by the PC as well as subjects indicated as screen failures, transient global amnesia (TGA) patients, and patients with unknown adjudicated diagnosis (i.e., a protocol violation or DWI-positive mimic). Following this QC review, N=575 samples were eligible for analysis. The diagnosis distribution among the 575 subjects was 414 (72%) ACVS and 161 (28%) Mimics.

Before the proteomic data were analyzed, they were cleaned and analyzed for statistical QC complications. Peptides that measured below or above the limit of quantification (BLOQ or ALOQ) and that were reported as NA were imputed following the same procedures used in Examples 3 and 4, as follows.

Any peptide measurements flagged as BLOQ or NA were imputed with half the minimum observed value.

All experimental samples for one peptide (DLIQHPK, SEQ ID NO: 4, vascular endothelial growth factor D) were BLOQ and were imputed to 0 prior to running the models.

Any peptide measurement flagged as ALOQ was imputed to 1.5 times the maximum observed value for that peptide.

Following imputation, peptide values were log 2 transformed and adjusted for plate and time effects (using the same procedure as in Examples 3 and 4; time was measured from symptom onset to blood draw in hours). The adjusted peptide measurements were then centered and scaled with the scale attributes from the peptides in Examples 3 and 4. Thrombospodin-1 was not measured using the same peptide as in Examples 3 and 4; therefore, it was imputed the mean using the Example 3 and 4 peptides to evaluate the GLM-16 model.

Performance of the Data

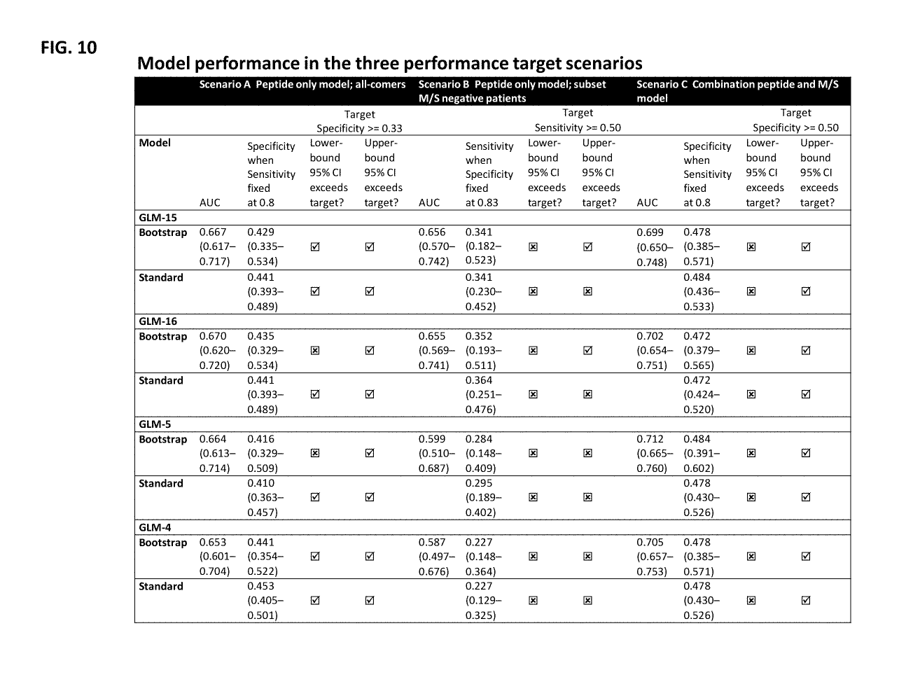

FIG. 10 shows the performance of the candidate models in the three target scenarios. The 95% CIs for performance estimates were computed using the following two methods.

1. Bootstrap: a bootstrap procedure with 2000 stratified bootstraps using the ‘pROC’ package.

2. Standard: the standard \hat{p}±1.96*se(\hat{p}) method, where \hat{p} is the sensitivity/specificity classification rule selected using a cut-point that achieves the minimum fixed value for the given target scenario and se(\hat{p})=sqrt((1−\hat{p})*\hat{p}/N) with N=# ACVS for the specificity and N=# Mimics for the sensitivity.

In Scenario A (replace M/S score for detection of ACVS), all four algorithms significantly exceeded the performance targets with the lower bound of the CI for specificity exceeding the specificity threshold 0.33 (with sensitivity held at a minimum of 0.80).

In Scenario B (upgrade stroke unit referral urgency for M/S-negative), the algorithms achieved sensitivities of approximately 0.35 for GLM-15 and GLM-16. The target sensitivity was 0.5 for specificity fixed at minimum of 0.83 in the subset of patients that were M/S negative.

In Scenario C (upgrade medical imaging urgency), the point estimate for specificity was similar to the target of 0.50 for all of the algorithms (i.e., specificity values of 0.472 to 0.484), and the upper bound of the 95% CI exceeded the target; thus, the target was achieved.

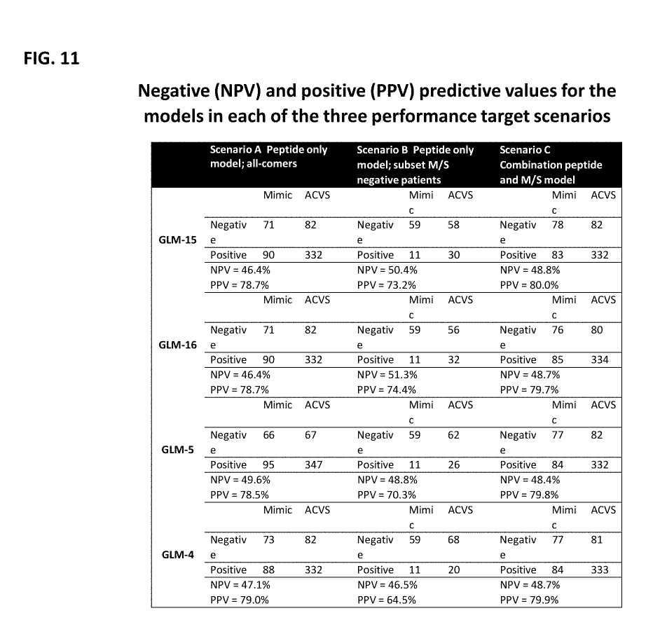

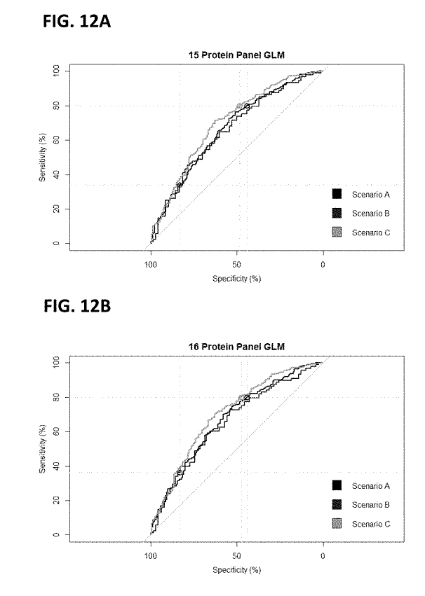

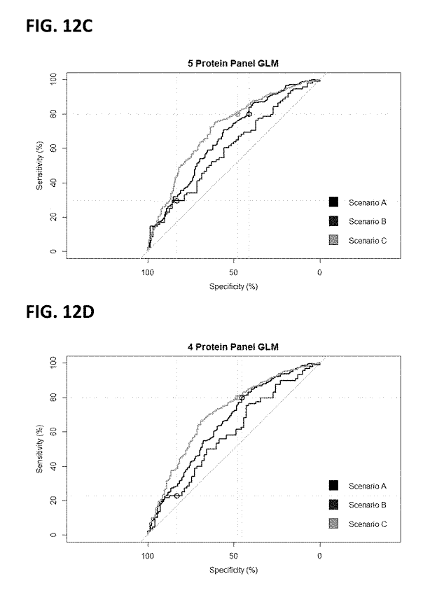

FIGS. 10-12 provide a full summary of performance measures, including the observed negative and positive predictive values for each of the algorithms under the three target scenarios. ROC curves are also provided.

TABLE 9
GLM-15 fit on standardized peptide data from
Examples 3 and 4

| | Peptide | | | | |
|---|---|---|---|---|---|
| | Se- | Esti- | Std. | z | Pr |
| Protein | quence | mate | Error | value | (>lzl) |
| (Intercept) | (Inter- | 1.046 | 0.108 | 9.686 | 0.0000 |
| | cept) | | | | |
| Adiponectin | GDPGLIGPK | 0.397 | 0.128 | 3.107 | 0.0019 |
| | (SEQ ID | | | | |
| | NO: 5) | | | | |
| Vascular | DLIQHPK | 0.333 | 0.109 | 3.066 | 0.0022 |
| endothelial | (SEQ ID | | | | |
| growth | NO: 4) | | | | |
| factor D | | | | | |
| Coagulation | SALVLQYLR | 0.557 | 0.196 | 2.842 | 0.0045 |
| factor IX | (SEQ ID | | | | |
| | NO: 16) | | | | |
| Heparin | TLEAQLTPR | −0.430 | 0.180 | −2.393 | 0.0167 |
| cofactor 2 | (SEQ ID | | | | |
| | NO: 12) | | | | |
| Myeloblastin | LVNVVLGVA | −0.258 | 0.109 | −2.360 | 0.0183 |
| | HNR | | | | |
| | (SEQ ID | | | | |
| | NO: 7) | | | | |
| L- | AEIEYLEK | −0.277 | 0.122 | −2.268 | 0.0233 |
| selectin | (SEQ ID | | | | |
| | NO: 2) | | | | |
| Coagulation | TGIVSGFGR | −0.374 | 0.175 | −2.131 | 0.0331 |
| factor X | (SEQ ID | | | | |
| | NO: 10) | | | | |
| Serum | ILIGTVFHK | −0.259 | 0.134 | −1.937 | 0.0528 |
| paraoxonase/ | (SEQ ID | | | | |
| lactonase 3 | NO: 8) | | | | |
| Hyaluronan- | DEIPHNDIA | −0.281 | 0.149 | −1.887 | 0.0591 |
| binding | LLK | | | | |
| protein 2 | (SEQ ID | | | | |
| | NO: 15) | | | | |
| Hemopexin | NFPSPVDAA | 0.315 | 0.186 | 1.693 | 0.0904 |
| | FR | | | | |
| | (SEQ ID | | | | |
| | NO: 6) | | | | |
| Plasma | AAAATGTIF | 0.186 | 0.126 | 1.473 | 0.1408 |
| serine | TFR | | | | |
| protease | (SEQ ID | | | | |
| inhibitor | NO: 11) | | | | |
| von | ILAGPAGDS | 0.149 | 0.116 | 1.291 | 0.1965 |
| Willebrand | NVVK | | | | |
| Factor | (SEQ ID | | | | |
| | NO: 13) | | | | |
| Coagulation | AEVDDVIQV | 0.213 | 0.173 | 1.229 | 0.2190 |
| factor V | R | | | | |
| | (SEQ ID | | | | |
| | NO: 9) | | | | |
| Apolipo- | FPEVDVLTK | 0.126 | 0.121 | 1.046 | 0.2957 |
| protein | (SEQ ID | | | | |
| B-100 | NO: 3) | | | | |
| Insulin- | FLNVLSPR | −0.051 | 0.141 | −0.362 | 0.7174 |
| like | (SEQ ID | | | | |
| growth | NO: 1) | | | | |
| factor- | | | | | |
| binding | | | | | |
| protein-1 | | | | | |

TABLE 10
GLM-15 + M/S fit on standardized peptide data
from Examples 3 and 4

| | Peptide | Esti- | Std. | z | Pr |
|---|---|---|---|---|---|
| Protein | Sequence | mate | Error | value | (>lzl) |
| (Motor/ | (Motor/ | 0.757 | 0.237 | 3.192 | 0.0014 |
| Speech) | Speech) | | | | |
| Adiponectin | GDPGLIGPK | 0.397 | 0.129 | 3.066 | 0.0022 |
| | (SEQ ID | | | | |
| | NO: 5) | | | | |
| Vascular | DLIQHPK | 0.334 | 0.110 | 3.034 | 0.0024 |
| endothelial | (SEQ ID | | | | |
| growth | NO: 4) | | | | |
| factor D | | | | | |
| Coagulation | SALVLQYLR | 0.551 | 0.199 | 2.768 | 0.0056 |
| factor IX | (SEQ ID | | | | |
| | NO: 16) | | | | |
| Myeloblastin | LVNVVLGAH | −0.277 | 0.109 | −2.529 | 0.0114 |
| | NVR | | | | |
| | (SEQ ID | | | | |
| | NO: 7) | | | | |
| Heparin | TLEAQLTPR | −0.456 | 0.182 | −2.507 | 0.0122 |
| cofactor 2 | (SEQ ID | | | | |
| | NO: 12) | | | | |
| (Intercept) | (Inter- | 0.481 | 0.203 | 2.371 | 0.0177 |
| | cept) | | | | |
| L-selectin | AEIEYLEK | −0.288 | 0.124 | −2.316 | 0.0206 |
| | (SEQ ID | | | | |
| | NO: 2) | | | | |
| Coagulation | TGIVSGFGR | −0.367 | 0.177 | −2.066 | 0.0388 |
| factor X | (SEQ ID | | | | |
| | NO: 10) | | | | |
| Hyaluronan- | DEIPHNDIA | −0.276 | 0.151 | −1.832 | 0.0669 |
| binding | LLK | | | | |
| protein 2 | (SEQ ID | | | | |
| | NO: 15) | | | | |
| Hemopexin | NFPSPVDAA | 0.341 | 0.189 | 1.805 | 0.0710 |
| | FR | | | | |
| | (SEQ ID | | | | |
| | NO: 6) | | | | |
| Serum | ILIGTVFHK | −0.228 | 0.135 | −1.688 | 0.0913 |
| paraoxonase/ | (SEQ ID | | | | |
| lactonase 3 | NO: 8) | | | | |
| Plasma | AAAATGTIF | 0.204 | 0.127 | 1.605 | 0.1085 |
| serine | TFR | | | | |
| protease | (SEQ ID | | | | |
| inhibitor | NO: 11) | | | | |
| Coagulation | AEVDDVIQV | 0.215 | 0.176 | 1.220 | 0.2224 |
| factor V | R | | | | |
| | (SEQ ID | | | | |
| | NO: 9) | | | | |
| von | ILAGPAGDS | 0.110 | 0.118 | 0.931 | 0.3517 |
| Willebrand | NVVK | | | | |
| Factor | (SEQ ID | | | | |
| | NO: 13) | | | | |
| Apolipo- | FPEVDVLTK | 0.111 | 0.123 | 0.901 | 0.3675 |
| protein | (SEQ ID | | | | |
| B-100 | NO: 3) | | | | |
| Insulin-like | FLNVLSPR | −0.063 | 0.143 | −0.444 | 0.6571 |
| growth | (SEQ ID | | | | |
| factor- | NO: 1) | | | | |
| binding | | | | | |
| protein-1 | | | | | |

TABLE 11
GLM-16 fit on standardized peptide data from
Examples 3 and 4

| | Peptide | Esti- | Std. | z | Pr |
|---|---|---|---|---|---|
| Protein | Sequence | mate | Error | value | (>lzl) |
| (Intercept) | (Inter- | 1.053 | 0.109 | 9.693 | 0.0000 |
| | cept) | | | | |
| Vascular | DLIQHPK | 0.351 | 0.110 | 3.198 | 0.0014 |
| endothelial | (SEQ ID | | | | |
| growth | NO: 4) | | | | |
| factor D | | | | | |
| Adiponectin | GDPGLIGPK | 0.383 | 0.128 | 2.981 | 0.0029 |
| | (SEQ ID | | | | |
| | NO: 5) | | | | |
| Coagulation | SALVLQYLR | 0.534 | 0.197 | 2.706 | 0.0068 |
| factor IX | (SEQ ID | | | | |
| | NO: 16) | | | | |
| L-selectin | AEIEYLEK | −0.301 | 0.124 | −2.429 | 0.0152 |
| | (SEQ ID | | | | |
| | NO: 2) | | | | |
| Heparin | TLEAQLTPR | −0.439 | 0.181 | −2.423 | 0.0154 |
| cofactor 2 | (SEQ ID | | | | |
| | NO: 12) | | | | |
| Myeloblastin | LVNVVLGAH | −0.252 | 0.110 | −2.296 | 0.0217 |
| | NVR | | | | |
| | (SEQ ID | | | | |
| | NO: 7) | | | | |
| Serum | ILIGTVFHK | −0.271 | 0.133 | −2.030 | 0.0424 |
| paraoxonase/ | (SEQ ID | | | | |
| lactonase 3 | NO: 8) | | | | |
| Hyaluronan- | DEIPHNDIA | −0.296 | 0.149 | −1.983 | 0.0474 |
| binding | LLK | | | | |
| protein 2 | (SEQ ID | | | | |
| | NO: 15) | | | | |
| Coagulation | TGIVSGFGR | −0.349 | 0.177 | −1.977 | 0.0480 |
| factor X | (SEQ ID | | | | |
| | NO: 10) | | | | |
| Hemopexin | NFPSPVDAA | 0.309 | 0.185 | 1.668 | 0.0954 |
| | FR | | | | |
| | (SEQ ID | | | | |
| | NO: 6) | | | | |
| Thrombo- | GPDPSSPAFR | 0.196 | 0.121 | 1.620 | 0.1052 |
| spondin-1 | (SEQ ID | | | | |
| | NO: 14) | | | | |
| Plasma | AAAATGTIFT | 0.177 | 0.124 | 1.427 | 0.1536 |
| serine | FR | | | | |
| protease | (SEQ ID | | | | |
| inhibitor | NO: 11) | | | | |
| Apolipo | FPEVDVLTK | 0.139 | 0.122 | 1.142 | 0.2533 |
| protein | (SEQ ID | | | | |
| B-100 | NO: 3) | | | | |
| von | ILAGPAGDSN | 0.131 | 0.116 | 1.123 | 0.2612 |
| Willebrand | VVK | | | | |
| Factor | (SEQ ID | | | | |
| | NO: 13) | | | | |
| Coagulation | AEVDDVIQVR | 0.182 | 0.175 | 1.036 | 0.3000 |
| factor V | (SEQ ID | | | | |
| | NO: 9) | | | | |
| Insulin- | FLNVLSPR | 0.036 | 0.151 | 0.238 | 0.8117 |
| like | (SEQ ID | | | | |
| growth | NO: 1) | | | | |
| factor- | | | | | |
| binding | | | | | |
| protein-1 | | | | | |

TABLE 12
GLM-16 + M/S fit on standardized peptide data
from Examples 3 and 4

| | Peptide | Esti- | Std. | z | Pr |
|---|---|---|---|---|---|
| Protein | Sequence | mate | Error | value | (>lzl) |
| Vascular | DLIQHPK | 0.351 | 0.111 | 3.160 | 0.0016 |
| endothelial | (SEQ ID | | | | |
| growth | NO: 4) | | | | |
| factor D | | | | | |
| (Motor/ | (Motor/ | 0.744 | 0.238 | 3.124 | 0.0018 |
| Speech) | Speech) | | | | |
| Adiponectin | GDPGLIGPK | 0.384 | 0.130 | 2.955 | 0.0031 |
| | (SEQ ID | | | | |
| | NO: 5) | | | | |
| Coagulation | SALVLQYLR | 0.531 | 0.200 | 2.651 | 0.0080 |
| factor IX | (SEQ ID | | | | |
| | NO: 16) | | | | |
| Heparin | TLEAQLTPR | −0.467 | 0.183 | −2.552 | 0.0107 |
| cofactor 2 | (SEQ ID | | | | |
| | NO: 12) | | | | |
| L-selectin | AEIEYLEK | −0.308 | 0.126 | −2.455 | 0.0141 |
| | (SEQ ID | | | | |
| | NO: 2) | | | | |
| Myelo- | LVNVVLGAH | −0.270 | 0.110 | −2.455 | 0.0141 |
| blastin | NVR | | | | |
| | (SEQ ID | | | | |
| | NO: 7) | | | | |
| (Intercept) | (Inter- | 0.496 | 0.204 | 2.434 | 0.0150 |
| | cept) | | | | |
| Coagulation | TGIVSGFGR | −0.343 | 0.179 | −1.921 | 0.0547 |
| factor X | (SEQ ID | | | | |
| | NO: 10) | | | | |
| Hyaluronan- | DEIPHNDIA | −0.290 | 0.151 | −1.920 | 0.0548 |
| binding | LLK | | | | |
| protein 2 | (SEQ ID | | | | |
| | NO: 15) | | | | |
| Hemopexin | NFPSPVDAA | 0.335 | 0.188 | 1.781 | 0.0748 |
| | FR | | | | |
| | (SEQ ID | | | | |
| | NO: 6) | | | | |
| Serum | ILIGTVFHK | −0.238 | 0.135 | −1.763 | 0.0778 |
| paraoxonase/ | (SEQ ID | | | | |
| lactonase 3 | NO: 8) | | | | |
| Plasma | AAAATGTIFT | 0.195 | 0.125 | 1.566 | 0.1173 |
| serine | FR | | | | |
| protease | (SEQ ID | | | | |
| inhibitor | NO: 11) | | | | |
| Thrombo- | GPDPSSPAFR | 0.182 | 0.123 | 1.486 | 0.1373 |
| spondin-1 | (SEQ ID | | | | |
| | NO: 14) | | | | |
| Coagulation | AEVDDVIQVR | 0.186 | 0.178 | 1.046 | 0.2954 |
| factor V | (SEQ ID | | | | |
| | NO: 9) | | | | |
| Apolipo- | FPEVDVLTK | 0.124 | 0.123 | 1.002 | 0.3162 |
| protein | (SEQ ID | | | | |
| B-100 | NO: 3) | | | | |
| von | ILAGPAGDSN | 0.094 | 0.119 | 0.790 | 0.4294 |
| Willebrand | VVK | | | | |
| Factor | (SEQ ID | | | | |
| | NO: 13) | | | | |
| Insulin-like | FLNVLSPR | 0.017 | 0.153 | 0.115 | 0.9088 |
| growth | (SEQ ID | | | | |
| factor- | NO: 1) | | | | |
| binding | | | | | |
| protein-1 | | | | | |

TABLE 13
GLM-5 fit on standardized peptide data from
Examples 3 and 4

| | Peptide | Esti- | Std. | z | Pr |
|---|---|---|---|---|---|
| Protein | Sequence | mate | Error | value | (>lzl) |
| (Inter- | (Inter- | 0.937 | 0.099 | 9.512 | 0.0000 |
| cept) | cept) | | | | |
| L- | AEIEYLEK | −0.265 | 0.108 | −2.451 | 0.0142 |
| selectin | (SEQ ID | | | | |
| | NO: 2) | | | | |
| Insulin- | FLNVLSPR | −0.202 | 0.116 | −1.747 | 0.0807 |
| like | (SEQ ID | | | | |
| growth | NO: 1) | | | | |
| factor- | | | | | |
| binding | | | | | |
| protein-1 | | | | | |
| Apolipo- | FPEVDVLTK | 0.182 | 0.106 | 1.715 | 0.0863 |
| protein | (SEQ ID | | | | |
| B-100 | NO: 3) | | | | |
| von | ILAGPAGDS | 0.169 | 0.106 | 1.597 | 0.1103 |
| Willebrand | NVVK | | | | |
| Factor | (SEQ ID | | | | |
| | NO: 13) | | | | |
| Coagulation | SALVLQYLR | 0.145 | 0.110 | 1.321 | 0.1865 |
| factor IX | (SEQ ID | | | | |
| | NO: 16) | | | | |

TABLE 14
GLM-5 + M/S fit on standardized peptide data
from Examples 3 and 4

| | Peptide | Esti- | Std. | z | Pr |
|---|---|---|---|---|---|
| Protein | Sequence | mate | Error | value | (>lzl) |
| (Motor/ | (Motor/ | 0.666 | 0.218 | 3.057 | 0.0022 |
| Speech) | Speech) | | | | |
| L-selectin | AEIEYLEK | −0.271 | 0.109 | −2.482 | 0.0131 |
| | (SEQ ID | | | | |
| | NO: 2) | | | | |
| (Intercept) | (Inter- | 0.445 | 0.185 | 2.402 | 0.0163 |
| | cept) | | | | |
| Insulin-like | FLNVLSPR | −0.202 | 0.117 | −1.727 | 0.0842 |
| growth | (SEQ ID | | | | |
| factor- | NO: 1) | | | | |
| binding | | | | | |
| protein-1 | | | | | |
| Apolipo- | FPEVDVLTK | 0.185 | 0.107 | 1.722 | 0.0851 |
| protein | (SEQ ID | | | | |
| B-100 | NO: 3) | | | | |
| Coagulation | SALVLQYLR | 0.143 | 0.111 | 1.291 | 0.1968 |
| factor IX | (SEQ ID | | | | |
| | NO: 16) | | | | |
| von | ILAGPAGDSN | 0.134 | 0.107 | 1.254 | 0.2099 |
| Willebrand | VVK | | | | |
| Factor | (SEQ ID | | | | |
| | NO: 13) | | | | |

TABLE 15
GLM-4 fit on standardized peptide data from
Examples 3 and 4

| | Peptide | Esti- | Std. | z | Pr |
|---|---|---|---|---|---|
| Protein | Sequence | mate | Error | value | (>lzl) |
| (Intercept) | (Inter- | 0.932 | 0.098 | 9.500 | 0.0000 |
| | cept) | | | | |
| Insulin-like | FLNVLSPR | −0.261 | 0.110 | −2.367 | 0.0179 |
| growth | (SEQ ID | | | | |
| factor- | NO: 1) | | | | |
| binding | | | | | |
| protein-1 | | | | | |
| L-selectin | AEIEYLEK | −0.242 | 0.107 | −2.264 | 0.0236 |
| | (SEQ ID | | | | |
| | NO: 2) | | | | |
| Apolipo- | FPEVDVLTK | 0.185 | 0.106 | 1.751 | 0.0799 |
| protein | (SEQ ID | | | | |
| B-100 | NO: 3) | | | | |
| Coagulation | SALVLQYLR | 0.165 | 0.108 | 1.521 | 0.1284 |
| factor IX | (SEQ ID | | | | |
| | NO: 16) | | | | |

TABLE 16
GLM-4 + M/S fit on standardized peptide data
from Examples 13 and 4

| | Peptide | Esti- | Std. | z | Pr |
|---|---|---|---|---|---|
| Protein | Sequence | mate | Error | value | (>lzl) |
| (Motor/ | (Motor/ | 0.697 | 0.216 | 3.220 | 0.0013 |
| Speech) | Speech) | | | | |
| L-selectin | AEIEYLEK | −0.254 | 0.108 | −2.347 | 0.0189 |
| | (SEQ ID | | | | |
| | NO: 2) | | | | |
| (Intercept) | (Inter- | 0.418 | 0.184 | 2.279 | 0.0227 |
| | cept) | | | | |
| Insulin-like | FLNVLSPR | −0.248 | 0.111 | −2.232 | 0.0256 |
| growth | (SEQ ID | | | | |
| factor- | NO: 1) | | | | |
| binding | | | | | |
| protein-1 | | | | | |
| Apolipo- | FPEVDVLTK | 0.188 | 0.107 | 1.753 | 0.0795 |
| protein | (SEQ ID | | | | |
| B-100 | NO: 3) | | | | |
| Coagulation | SALVLQYLR | 0.157 | 0.110 | 1.436 | 0.1510 |
| factor IX | (SEQ ID | | | | |
| | NO: 16) | | | | |

### Example 8

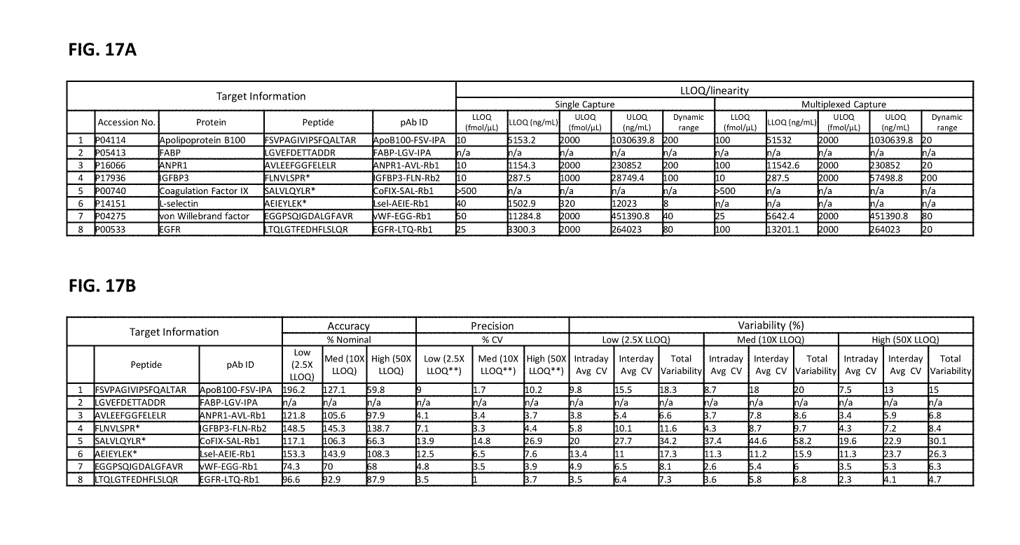

This example illustrates a summary of a validation experiment for an iMALDI panel (see FIGS. 17A-17C and TABLE 17).

The multiplexed immuno-MALDI (iMALDI) assay was validated following the minimum guidelines set out by the Clinical Proteomic Tumor Analysis Consortium (CPTAC) Assay Development Working Group (Whiteaker et al., 2014, incorporated herein by reference). FIGS. 17A-17B indicate the results for the performance of the assay in regards to accuracy and precision over the linear response range for the assy.

Lower Limit of Quantitation (LLOQ) and linearity were determined by triplicate capture curves spanning two-orders of magnitude (100×). Curves were constructed by spiking a constant concentration of synthetic Stable Isotope Standard (SIS) peptide and varying concentrations of synthetic light peptide into a pool of trypsin digested Escherichia coli. The LLOQ was defined as the lowest point on the curve with both average precision and accuracy <20% Coefficient of Variation (CV). LLOQ and linearity were determined for both the single and multiplexed iMALDI assays.

Evaluating assay accuracy was performed by analyzing five replicate iMALDI captures on five separate days at three concentration levels configured within the dynamic range of the assay. To qualify as accurate, the percent nominal accuracy at each concentration level must be <20%.

Precision was evaluated by ensuring the % CV for replicate captures from samples with variable, known concentrations of synthetic light peptide was <20% for each of concentration level.

Assay variability estimated the reproducibility at three concentrations covering the linear range. Five matrix digestion and iMALDI captures were prepared five times per day (intra-assay variability) as well as on five different days (inter-assay variability). An assay was acceptable when all concentrations evaluated had a total CV of <20% (calculated by the square root of the sum of squares of the intra- and inter-assay accuracy).

### REFERENCES

Pope, R., Malmstrom, D., Chambers, A. G., et al. (2015). An automated assay for the clinical measurement of plasma renin activity by immune-MALDI 9iMALDI). Biochimica et Biophysica Acta, 1854, 547-558.
Whiteaker J. R., Halusa G. N., Hoofnagle A. N., et al. (2014). CPTAC Assay Portal: a repository of targeted proteomic assays. Nature Methods, 11(7), 703-704.

TABLE 17
Exemplary iMALDI proteins with anti-
peptide polyclonal antibodies:

| Uniprot Accession No. | Protein Name |
|---|---|
| P04114 | Apolipoprotein B100 |
| P05413 | FABP3 |
| P16066 | ANPR1 |
| P17936 | IGFBP3 |
| P00740 | Coagulation Factor IX |
| P14151 | L-selectin |
| P04275 | von Willebrand factor |
| P00533 | EGFR |

### Example 9

### Clinical Use

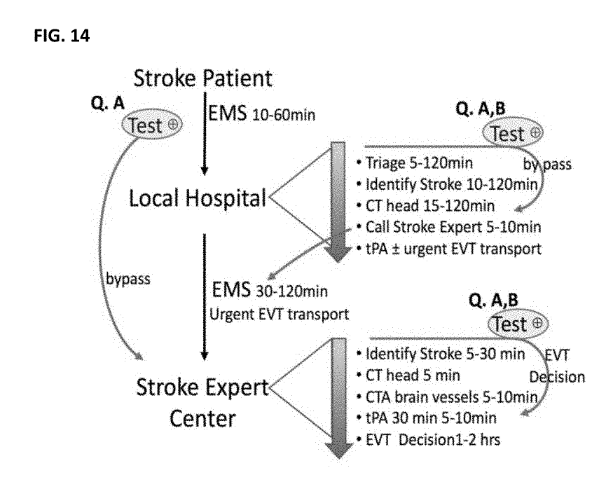

The methods described herein are used in a clinical setting provide a diagnosis or prognosis as well as triage patients to the closest tPA-capable hospital or stroke expert center for tPA and endovascular thrombectomy (EVT) as shown in FIG. 14. In this example, the methods herein are used in hospitals with computerized tomography (CT) to aid in deciding if a patient requires urgent transport for an EVT or should stay and receive tPA (FIG. 14).

In view of the many possible embodiments to which the principles of the disclosure may be applied, it should be recognized that illustrated embodiments are only examples of the disclosure and should not be considered a limitation on the scope of the invention. Rather, the scope of the invention is defined by the following claims. We therefore claim as our invention all that comes within the scope and spirit of these claims.

We claim:

1. A method of treating a subject with acute cerebrovascular syndrome (ACVS), comprising:
measuring at least two ACVS-related peptides derived from proteins in a sample obtained from a subject, wherein the at least two ACVS-related proteins comprise at least two of fatty acid binding protein 3 (FABP3), atrial natriuretic peptide receptor-1 (ANPR-1), insulin-like growth factor binding protein 3 (IGFBP-3), coagulation factor IX (F9), L-selectin (SELL), apolipoprotein B100 (apoB100), Vascular endothelial growth factor D (VEGF-D), adiponectin (ADPN), von Willebrand factor (vWF), thrombospondin-1 (THBS1), prolactin (PRL), serum paraoxonase 3 (PON3), epidermal growth factor receptor (EGFR), hemopexin (HPX), myeloblastin (MBT), coagulation factor V (F5), coagulation factor X (F10), plasma serine protease inhibitor (SERPIN A5), heparin cofactor 2 (HCII), and hyaluronan-binding protein 2 (HABP2);
measuring differential expression of the at least two ACVS-related proteins compared to a control representing expression for each of the at least two ACVS-related proteins expected in a sample from a subject who does not have ACVS; and
administering a therapeutically effective amount of at least one of thrombolytic therapy, antiplatelet therapy, anticoagulant therapy, or surgery to the subject with ACVS, thereby treating the subject.

2. The method of claim 1, wherein the subject with ACVS has transient ischemic attack (TIA), and the ACVS-related proteins are TIA-related proteins.

3. The method of claim 1, wherein the at least two ACVS-related proteins comprise FABP3, ANPR-1, IGFBP-3, F9, SELL, and apoB100.

4. The method of claim 3, wherein the at least two ACVS-related proteins further comprise at least one of ADPN, vWF, THBS1, PON3, EGFR, VEGF-D, PRL, adiponectin, HPX, MBT, F5, F10, SERPIN A5, HCII, and HABP2.

5. The method of claim 1, wherein the at least two ACVS-related proteins comprise:
IGFBP-3, F9, SELL, apoB100, ADPN, vWF, THBS1, PON3, VEGF-D, HPX, MBT, F5, F10, SERPIN A5, HCII, and HABP2;
IGFBP-3, F9, SELL, apoB100, ADPN, vWF, PON3, VEGF-D, HPX, MBT, F5, F10, SERPIN A5, HCII, and HABP2; IGFBP-3, F9, SELL, apoB100, and vWF;
IGFBP-3, F9, SELL, and apoB100;
apoB100, FABP3, ANPR-1, IGFBP-3, F9, SELL, vWF, and EGFR;
apoB100, ANPR-1, IGFBP-3, F9, SELL, vWF, and EGFR;
apoB100, ANPR-1, IGFBP-3, SELL, vWF, and EGFR; or
apoB100, ANPR-1, IGFBP-3, vWF, and EGFR.

6. The method of claim 5, wherein the presence of motor weakness, aphasia, and/or dysarthria in the subject is unknown and/or is not considered prior to performing the method.

7. The method of claim 5, wherein motor weakness, aphasia, and/or dysarthria is not present in the subject.

8. The method of claim 5, further comprising considering whether motor weakness, aphasia, and/or dysarthria is present in the subject, wherein the presence of motor weakness, aphasia, and/or dysarthria in the subject is known.

9. The method of claim 1, wherein measuring the at least two ACVS-related peptides uses mass spectrometry.

10. The method of claim 9, wherein the mass spectrometry comprises an immuno matrix-assisted laser desorption/ionization (iMALDI) assay or a multiple reaction monitoring (MRM) assay.

11. The method of claim 10, wherein the iMALDI assay is used with polyclonal or monoclonal antibodies.

12. The method of claim 10, wherein the MRM assay is an enriched MRM assay.

13. The method of claim 1, wherein the ACVS-related peptides are derived from proteins by using a protease.

14. The method of claim 13, where in the protease is at least one of trypsin, chymotryptsin, endoprotease Glu-C, endoprotese Lys-C, endoprotease AspN, elastinase, pepsin, and endoprotease Arg-C.

15. The method of claim 1, wherein the peptides comprise or consist of the peptides listed in FIG. 5.

16. The method of claim 1, wherein:
IGFBP3 is measured by detecting SEQ ID NO: 1;
SELL is measured by detecting SEQ ID NO: 2;
apoB100 is measured by detecting SEQ ID NO: 3 and/or SEQ ID NO: 17;
VEGF-D is measured by detecting SEQ ID NO: 4;
ADPN is measured by detecting SEQ ID NO: 5;
HPX is measured by detecting SEQ ID NO: 6;
MBT is measured by detecting SEQ ID NO: 7;
PON3 is measured by detecting SEQ ID NO: 8;
F5 is measured by detecting SEQ ID NO: 9;
F10 is measured by detecting SEQ ID NO: 10
SERPINAS is measured by detecting SEQ ID NO: 11;
HCF2 is measured by detecting SEQ ID NO: 12;
vWF is measured by detecting SEQ ID NO: 13;
THBS1 is measured by detecting SEQ ID NO: 14;
HABP2 is measured by detecting SEQ ID NO: 15;
F9 is measured by detecting SEQ ID NO: 16;
FABP3 is measured by detecting SEQ ID NO: 18; and/or
ANPR-1 is measured by detecting SEQ ID NO: 19.

17. The method of claim 1, wherein the expression is measured using a multiplex assay or an individual assay for each protein or peptide.

18. The method of claim 1, wherein the subject is human.

19. The method of claim 1, wherein the sample is a biological sample, tissue sample, or biological fluid sample.

20. The method of claim 1, wherein the sample is a blood sample.

21. The method of claim 1, wherein the sample is plasma, whole blood, serum, or dried blood spots.
